(19) United States
(12) Patent Application Publication
(10) Pub. No.: US 2023/0183678 A1
(43) Pub. Date: Jun. 15, 2023

(54) IN-CELL CONTINUOUS TARGET-GENE EVOLUTION, SCREENING AND SELECTION

(71) Applicant: COMMISSARIAT À L'ENERGIE ATOMIQUE ET AUX ENERGIES ALTERNATIVES, PARIS (FR)

(72) Inventors: OSCAR PEREIRA RAMOS, GIF-SUR-YVETTE CEDEX (FR); LOÏC MARTIN, GIF-SUR-YVETTE CEDEX (FR)

(21) Appl. No.: 17/925,840

(22) PCT Filed: May 19, 2021
(86) PCT No.: PCT/EP2021/063247

§ 371 Date: Nov. 17, 2022

(30) Foreign Application Priority Data
May 20, 2020 (EP) 20305531.4

(51) Int. Cl. C12N 15/10; C12N 15/70

(52) U.S. Cl. CPC C12N 15/1058; C12N 15/1055; C12N 15/70; C07K 2319/80; C07K 2319/85

(57) ABSTRACT

The present invention relates to methods and means for implementing evolution of a gene of interest inside bacterial cells.

### FIELD OF THE INVENTION

The present invention relates to methods and means for evolution of a target sequence of interest.

### BACKGROUND OF THE INVENTION

Current molecular evolution methods, mainly committed to binders engineering, such as display technologies, impose a series of constraints such as: 1) The high cost and time per optimization cycle related to the library construction using purified reagents including molecular biology products, target protein production (expression, purification and labeling), biopanning method development and man-hours; 2) Limited diversity imposed by the cell transformation bottleneck; 3) Experimenter bias and; 4) Due to the mentioned constraints, these methods frequently impose to focus the diversity to small regions of the evolving molecule thus requiring previous structure and function knowledge, making difficult to implement multiple evolution rounds, to scale-up and to parallelize the assays.

State-of-art technologies that comply to the continuous evolution paradigm such as PACE (Esvelt et al, 2011) and MAGE (Wang et al. 2009) can partially address some of these constraints by using specially conceived electronic apparatus that are not commercially available and impose evident hurdles to assay parallelization and scale-up.

The present invention is aiming to provide improved methods overcoming the mentioned drawbacks.

### SUMMARY OF THE INVENTION

The present invention provides methods and means for implementing evolution inside cells. It should allow to address some major concerns in protein engineering projects such as: a) the limitations regarding the diversity up-scale, b) the requirement of highly optimized in vitro reaction using purified products by experts in the field of molecular biology and molecular display, c) the associated costs, d) the high time-to-results and, e) the relative low convenience of display based methods.

In other words, the invention concerns methods and means that implement an intracellular continuous evolution program focused on one (or multiple) target-gene(s) and that may encompass all the required evolutionary steps: Diversity generation, variant production and optionally screening of protein variants and stopping the generation of diversity if a good variant is found. This new technology should then allow to:
1. simplify molecular evolution by suppressing several steps requiring experimenter's intervention.
2. reduce cost, time and experimenter bias since no in vitro reaction would be required after cell transformation.
3. overcome the current diversity size limitations associated to the cell transformation bottleneck since the diversity should be generated inside cells. As a consequence, the number of independent clones can be modulated simply by adjusting the culture volume.
4. increase the diversity of solutions (good variants) since every cell implied in a continuous evolution process could generate variants resulting from different evolutionary pathways (theoretically, each cell is converted into an independent gene evolution machine).
5. avoid specific device-related constraints for execution, thereby, limiting the investment required for its use and granting easiness for technology application, scale-up and parallelization.
6. obviate the need of purified target-protein and library construction required for most display-based strategies.

In a particular aspect, the present invention relates to a method for generating diversity in a gene L, comprising:
providing a bacterial cell comprising a molecular complex formed by the association of:
a scaffold protein (SP),
a template RNA (tpRNA) comprising from 5′ to 3′: the gene L, an RTtag sequence operably linked to the gene L and a scaffold protein binding module 1 (SPBM1) sequence capable of binding to the SP at a first specific binding site (SPS1).
a primer RNA (prRNA) comprising: an RTprimer sequence positioned in 3′ end of the prRNA and capable of complementary pairing to the RTtag sequence, a scaffold protein binding module 2 (SPBM2) sequence capable of binding to the SP at a second specific binding site (SPS2) and a reverse transcriptase binding module (RBM) sequence, and
a fusion protein (RBD-RT) comprising a reverse transcriptase (RT) and an RBM binding domain (RBD) capable of binding to the RBM of the prRNA; and
placing the bacterial cell in conditions that allow the reverse transcription of the gene L, thereby generating altered copies of said gene L of the tpRNA.

Optionally, the RT of the fusion protein is TF1 or the HIV or MMLV reverse transcriptase.

Optionally, the SP is Hfq protein or a fragment or variant thereof.

Optionally, the prRNA further comprises a transfer RNA (tRNA) sequence contiguously positioned 3′ upstream of the RTprimer sequence, said tRNA sequence comprising a specific site that can be cleaved by a bacterial cell RNAse, preferably by RNAse P, thereby producing a well-defined 3′ prRNA end and a tRNA.

Preferably, the bacterial cell further expresses a homologous recombination (HR) factor capable of integrating the altered copies of the gene L into a DNA vector or into a genome of the bacterial cell, said vector or genome comprising a copy of the gene L, thereby preserving the altered copies of the gene L from degradation and allowing it to be expressed or to be iteratively altered in new cycles. Optionally, the HR factor is a lambda phage beta protein (λBet).

Optionally, the bacterial cell further expresses a preservative effector capable of inhibiting an RNAse, thereby preserving tpRNA, prRNA and altered copies of the gene L from degradation by RNAse. Optionally, the preservative effector is RNA helicase rhlB or a fragment 711-844 of RNAse E.

Alternatively, the bacterial cell further expresses a preservative effector capable of impairing the mismatch repair system (MMR) function. Optionally, the preservative effector is a deoxyadenosine methylase (dam), preferably a dam over-expressed by transient methods, or mutL and/or mutS dominant negative mutants.

The present invention also relates to a method for screening a ligand molecule capable of binding a target molecule from variants encoded by altered copies of a gene L prepared by the method according to the present invention, wherein the bacterial cell further comprises a bacterial two-hybrid system (B2H) comprising:
a promoter (P), a sequence defining a ribosome binding site (RBS) and a reporter gene, the P sequence being operably linked to the RBS sequence and the reporter gene,

and
a fusion protein (FPR) comprising the target molecule and a DNA binding domain (DBD), said DBD being capable of binding to a site located at proximity of the promoter P so as to promote the expression of the reporter gene when the target molecule is bound to a variant encoded by an altered copy of the gene L, and
a fusion protein (FPL) comprising a variant encoded by an altered copy of the gene L and transcription subunits (TrSu) capable of recruiting an RNA polymerase,

or
a fusion protein (FPL) comprising a variant encoded by an altered copy of the gene L and a DNA binding domain (DBD), said DBD being capable of binding to a site located at proximity of the promoter P so as to promote the expression of the reporter gene when the target molecule is bound to a variant encoded by an altered copy of the gene L,
a fusion protein (FPR) comprising the target molecule and transcription subunits (TrSu) capable of recruiting an RNA polymerase,

and

the method comprises the selection of the variant encoded by an altered copy of the gene L when the reporter is expressed, optionally at least at a predetermined level.

Optionally, the B2H further comprises a DNA invertase gene operably linked to the promoter P, said DNA invertase being capable of targeting DNA invertase sites that flank DNA sequences encoding the RT and/or the HR factor, thereby stopping the method for generating diversity in a gene L once the binding between the target molecule and the ligand molecule occurs.

Alternatively, the DNA invertase could be replaced by highly specific restriction enzyme (such as SceI) and by replacing invertase sites by the corresponding restriction sites. In this aspect, the B2H further comprises a gene encoding a highly specific restriction enzyme (such as SceI) to the promoter P, said restriction enzyme being capable of introducing double-stranded break at restriction sites that flank DNA sequences encoding the RT and/or the HR factor, thereby stopping the method for generating diversity in a gene L once the binding between the target molecule and the ligand molecule occurs, in particular by removal of the DNA sequences encoding the RT and/or the HR factor.

In another alternative, the method for generating diversity in the gene L can be stopped by using a transcription repressor. In this aspect, the B2H further comprises a gene encoding a transcription repressor to the promoter P or P′, said transcription repressor being capable of stopping the expression of the DNA sequences encoding the RT and/or the HR factor, thereby stopping the method for generating diversity in a gene L once the binding between the target molecule and the ligand molecule occurs.

Optionally, the expression of the FPR and/or FPL component, for instance the component comprising the DBD, is controlled by the association of a strong promoter and a weak RBS.

The present invention further relates to a method for screening a ligand molecule that loses the capacity of binding a target molecule from variants encoded by altered copies of a gene L prepared by the method according to the present invention, wherein the bacterial cell further comprises a B2H system comprising:
a first promoter P, a sequence defining a first ribosome binding site (RBS) and a reporter gene, the first promoter P being operably linked to the first RBS sequence and the reporter gene and allowing a stable basal level of expression of the reporter gene, and
a second promoter P′, a sequence defining a second RBS and a repressor gene, the second promoter P′ being operably linked to the second RBS sequence and the repressor gene, said repressor being capable of targeting the first promoter P to block the transcription of the reporter gene,
a fusion protein (FPR) and fusion protein (FPL), wherein the fusion protein (FPR) comprises the target molecule and a DNA binding domain (DBD), said DBD being capable of binding to a site located at proximity of the promoter P′ so as to promote the expression of the repressor gene when the target molecule is bound to a variant encoded by an altered copy of the gene L, and the fusion protein (FPL) comprises a variant encoded by an altered copy of the gene L and transcription subunits (TrSu) capable of recruiting an RNA polymerase; or wherein the fusion protein (FPR) comprises the target molecule and transcription subunits (TrSu) capable of recruiting an RNA polymerase, and the fusion protein (FPL) comprising a variant encoded by an altered copy of the gene L and a DNA binding domain (DBD), said DBD being capable of binding to a site located at proximity of the promoter P′ so as to promote the expression of the repressor gene when the target molecule is bound to a variant encoded by an altered copy of the gene L; and the method comprises the selection of the variant encoded by an altered copy of the gene L when the expression of the reporter is increased, optionally at least at a predetermined level.

Optionally, the B2H further comprises a DNA invertase gene operably linked to the second promoter P′, said DNA invertase being capable of targeting DNA invertase sites that flank DNA sequences encoding the RT and/or the HR factor, thereby stopping the method for generating diversity in a gene L once the binding between the target molecule and the ligand molecule is lost.

Optionally, the B2H further comprises a gene encoding a highly specific restriction enzyme to the promoter P′, said restriction enzyme being capable of introducing double-stranded break at restriction sites that flank DNA sequences encoding the RT and/or the HR factor, thereby stopping the method for generating diversity in a gene L once the binding between the target molecule and the ligand molecule is lost, in particular by removal of the DNA sequences encoding the RT and/or the HR factor.

Optionally, the B2H further comprises a gene encoding a transcription repressor to the promoter P′, said transcription repressor being capable of stopping the expression of the DNA sequences encoding the RT and/or the HR factor, thereby stopping the method for generating diversity in a gene L once the binding between the target molecule and the ligand molecule is lost. Optionally, the repressor under the control of the second promoter P′ is capable of stopping the expression of the DNA sequences encoding the RT and/or the HR factor.

Optionally, the expression of the FPR and/or FPL component, for instance the component comprising the DBD, is controlled by the association of a strong promoter and a weak RBS.

In addition, the present invention relates to a single vector or a set of vectors that can be transformed in a bacterial cell, comprising:
a transcription cassette (tC1) comprising a sequence encoding a pre-tpRNA operably linked to a promoter (P1), said pre-tpRNA comprising from 5′ to 3′: an insertion site suitable for the insertion of a gene L, an RTtag sequence operably linked to the gene L to be inserted and a SPBM1 sequence, wherein said tC1 is suitable for allowing, in the bacterial cell, the transcription of a tpRNA including an inserted gene L, wherein the SPBM1 is capable of binding to an SP present in the bacterial cell at a first specific binding site (SPS1).
a transcription cassette (tC2) comprising a sequence encoding a prRNA operably linked to a promoter (P2), said prRNA comprising: an RBM sequence positioned in 5′ end, an SPBM2 sequence and an RTprimer, wherein said tC2 is suitable for allowing, in the bacterial cell, the transcription of a prRNA, wherein the RTprimer is capable of complementary pairing to the RTtag, the SPBM2 is capable of binding to the SP at a second specific binding site (SPS2), and
an expression cassette (eC1) comprising a sequence encoding an RBD-RT fusion protein operably linked to a promoter (P3), said RBD-RT comprising a reverse transcriptase (RT) sequence and an RBD sequence, wherein said eC1 is suitable for allowing, in the bacterial cell, the expression of the RBD-RT fusion protein, wherein the RBD is capable of binding to the RBM of prRNA.

Optionally, the single vector or the set of vectors further comprises an expression cassette (eC2) comprising a sequence encoding the SP operably linked to a promoter (P4), preferably said SP being the Hfq protein, wherein eC2 is suitable for allowing, in the bacterial cell, the expression of the SP, preferably the Hfq protein.

Optionally, in the single vector or the set of vectors, the sequence encoding the prRNA further comprises a sequence encoding a tRNA sequence contiguously positioned downstream of the RTprimer sequence, a site cleavable by an RNAse of the bacterial cell is present between the said tRNA sequence and said RTprimer, thereby allowing the production of a well-defined 3′ prRNA end.

Optionally, the single vector or the set of vectors further comprises an expression cassette (eC3) comprising an HR factor gene operably linked to a promoter (P5), wherein said eC3 is suitable for allowing, in the bacterial cell, the expression of an HR factor capable of integrating the altered copies of the gene L into a DNA vector or into the genome of the bacterial cell, said vector or genome comprising a copy of the gene L, thereby preserving the altered copies of the gene L from degradation and allowing it to be expressed or to be iteratively altered in new cycles.

Optionally, the single vector or the set of vectors further comprises:
an expression cassette (eC4) comprising a sequence encoding a reporter gene operably linked to a promoter (P6),
an expression cassette (eC5) comprising a sequence encoding an FPR protein operably linked to a promoter (P7), said FPR comprising a target domain and a DBD sequence, said DBD being capable of binding to a site located at proximity of the promoter P6 so as to promote the expression of the reporter gene when the target molecule is bound to a variant encoded by an altered copy of the gene L, wherein said eC5 is suitable for allowing, in the bacterial cell, the expression of an FPR protein, and
an expression cassette (eC6) comprising a sequence encoding an FPL protein operably linked to a promoter (P8), said FPL comprising an insertion site suitable for the insertion of the gene L and transcription subunits (TrSu) capable of recruiting an RNA polymerase, wherein said eC6 is suitable for allowing, in the bacterial cell, the expression of a FPL protein comprising either a ligand encoded by the gene L or a variant thereof encoded by an HR-integrated altered copy of gene L;
or
an expression cassette (eC4) comprising a sequence encoding a reporter gene operably linked to a promoter (P6),
an expression cassette (eC5) comprising a sequence encoding an FPR protein operably linked to a promoter (P7), said FPR comprising a target domain and transcription subunits (TrSu) capable of recruiting an RNA polymerase, wherein said eC5 is suitable for allowing, in the bacterial cell, the expression of an FPR protein, and
an expression cassette (eC6) comprising a sequence encoding an FPL protein operably linked to a promoter (P8), said FPL comprising an insertion site suitable for the insertion of the gene L and a DBD sequence, said DBD being capable of binding to a site located at proximity of the promoter P6 so as to promote the expression of the reporter gene when the target molecule is bound to a variant encoded by an altered copy of the gene L, wherein said eC6 is suitable for allowing, in the bacterial cell, the expression of an FPL protein comprising either a ligand encoded by the gene L or a variant thereof encoded by an HR-integrated altered copy of gene L;
or
an expression cassette (eC4) comprising a sequence encoding a repressor gene operably linked to a promoter (P6),
an expression cassette (eC4′) comprising a sequence encoding a reporter gene operably linked to a promoter (P6′), the expression of the reporter gene being negatively controlled by the repressor encoded by (eC4),
an expression cassette (eC5) comprising a sequence encoding an FPR protein operably linked to a promoter (P7), said FPR comprising a target domain and a DBD sequence, said DBD being capable of binding to a site located at proximity of the promoter P6 so as to promote the expression of the repressor gene when the target molecule is bound to a variant encoded by an altered copy of the gene L, wherein said eC5 is suitable for allowing, in the bacterial cell, the expression of an FPR protein, and
an expression cassette (eC6) comprising a sequence encoding an FPL protein operably linked to a promoter (P8), said FPL comprising an insertion site suitable for the insertion of the gene L and transcription subunits (TrSu) capable of recruiting an RNA polymerase, wherein said eC6 is suitable for allowing, in the bacterial cell, the expression of a FPL protein comprising either a ligand encoded by the gene L or a variant thereof encoded by an HR-integrated altered copy of gene L;
or
an expression cassette (eC4) comprising a sequence encoding a repressor gene operably linked to a promoter (P6),
an expression cassette (eC4′) comprising a sequence encoding a reporter gene operably linked to a promoter (P6′), the expression of the reporter gene being negatively controlled by the repressor encoded by (eC4),
an expression cassette (eC5) comprising a sequence encoding an FPR protein operably linked to a promoter (P7), said FPR comprising a target domain and transcription subunits (TrSu) capable of recruiting an RNA polymerase, wherein said eC5 is suitable for allowing, in the bacterial cell, the expression of an FPR protein, and
an expression cassette (eC6) comprising a sequence encoding an FPL protein operably linked to a promoter (P8), said FPL comprising an insertion site suitable for the insertion of the gene L and a DBD sequence, said DBD being capable of binding to a site located at proximity of the promoter P6 so as to promote the expression of the repressor gene when the target molecule is bound to a variant encoded by an altered copy of the gene L, wherein said eC6 is suitable for allowing, in the bacterial cell, the expression of an FPL protein comprising either a ligand encoded by the gene L or a variant thereof encoded by an HR-integrated altered copy of gene L.

Optionally, the eC1 further comprises DNA invertase sites flanking the sequence encoding RBD-RT and/or the eC3 further comprises DNA invertase sites flanking the sequence encoding HR factor gene, and the eC4 further comprises a sequence encoding a DNA invertase gene operably linked to P6. Optionally, the eC1 further comprises restriction sites flanking the sequence encoding RBD-RT and/or the eC3 further comprises restriction sites flanking the sequence encoding HR factor gene, and the eC4 further comprises a sequence encoding a restriction enzyme gene operably linked to P6. Optionally, the eC1 further comprises a sequence encoding a transcription repressor gene operably linked to P6, and the expression of the sequence encoding RBD-RT of the eC1 and/or the sequence encoding HR factor gene of the eC3 can be stopped by said transcription repressor gene.

Optionally, the tC1 and eC6 comprise a gene L instead of the insertion sites.

Optionally, said vectors are low copy vectors.

Finally, the present invention relates to a bacterial cell comprising said single vector or set of vectors and the use thereof for implementing evolution of a gene of interest.

The present invention further relates to an improved B2H system and its uses.

### BRIEF DESCRIPTION OF THE FIGURES

FIG. 1: Schematic representation of the basic concepts behind one implementation of the intracellular system for targeted and continuous gene evolution. RNAs transcribed from the evolving gene are reverse transcribed and mutations are randomly incorporated. The mutated DNA replaces the original copy of the gene by homologous recombination. Dynamically, different protein variant fusions are expressed and one of them interact conveniently with the target fusion, hence, triggering the expression of reporter, marker and evolution arrest genes which signalize that a good binder was produced and stop continuous evolution.

FIG. 2: RT (1), HR (2), two-hybrid (3) and system arrest (4) modules interaction. With the intent to facilitate the comprehension of the proposed artificial biological circuit, one possible embodiment is schematized and corresponds to the evolution of protein ligands against a target protein.

(A) Semantic connection among modules. The reverse transcription module (1) converts the RNA of an evolving binder into a mutated ssDNAs or dsDNAs. Homologous recombination module (2) replaces the original gene (or part of the gene) by the mutated version encoded in ssDNAs or dsDNAs thereby, allowing the variant to be expressed. The two-hybrid module (3) screens the produced variants and if a strong enough binder is found a signal is triggered in order to arrest module 1 and 2 (module 4), as well as, a signal allowing the isolation of the corresponding cell. Therefore, diversity generation stops but not the expression of the selected variant and its detection by module 3, thus, allowing the isolation of the corresponding cell, the identification of the evolving variant and, therefore, its characterization by current techniques.

(B) Detailed molecular connections (DNA, RNA and protein levels) of one possible evolutionary strategy for protein binders. Target gene (gene T) fused to a DNA binding domain (DBD) coding region is transcribed and translated. The protein fusion T-DBD recognizes a specific motif on the DNA. The ligand gene to be evolved (gene L) can be transcribed from a fusion with a sequence that should allow reverse transcription, here named RTtag. Low-fidelity conversion of the RNA into DNA generates gene variants (module 1) that replaces (module 2) the original copy of gene L. Gene L (or its variants) fused to transcription subunits or transcription activator (TrSu) are expressed and if one of them interacts with the target gene in a stable enough way it triggers (module 3) the expression of interaction signals (for instance but not limited to: luminescent/fluorescent proteins, enzymes, auxotrophic markers, antibiotic resistance markers, etc) as well as signals to arrest modules 1 and 2 (for instance but not limited to: restriction enzymes, recombinases, transposases, repressors, etc). DNA is represented by double lines, RNA by single lines, protein domains by distinct geometric forms.

FIG. 3: Scheme of the genetic system designed to demonstrate the feasibility of coupling reverse transcription (RT) with homologous recombination (HR).

(A) The reverse transcription enzyme (RT) and the recombination factor (λ Bet) are expressed from one plasmid (up, left; VN575). KanOn RNA precursor containing an intron is transcribed from the same plasmid (bottom, left) and spontaneously gives rise to the self-spliced KanOn RNA. The later RNA form is recognized by an intracellular oligonucleotide (RT primer) and the hybridized oligonucleotides are used by RT enzyme to synthesize KanOn cDNA which, in turn, associates with λ Bet protein to patch the internal stop codon region of KanOff gene in the other plasmid (up, right; VN591) by homologous recombination. Thus, the initial KanOff gene is converted to a functional version (KanOn gene), the cells become resistant to kanamycin and can be conveniently isolated and sequenced. DNA is represented by double lines, RNA by pointed single lines, RT primer oligonucleotide by a gray pointed line and cDNA by a full line. Stop codons are indicated by “Stop” symbols. Transcription promoters are represented by arrows to the right and transcription terminator as “T”.

(B) Plasmid harboring the KanOff gene (VN591), a non-functional kanamycin resistance gene generated by the introduction of a stop codon at the 5′ coding region between td exon bases.

(C) Plasmid containing an RT enzyme, λ bet protein and KanOn gene with td intron insertion (VN575). The constitutive expression of tetR allows the regulation of expression from pLtetO promoter and, consequently, the intracellular amount of the bicistronic RNA that codes for RT and λ Bet.

FIG. 4: Generalization of the improved RT module by co-localization of the RNA corresponding to the evolving gene, RNA primer and reverse transcriptase enzyme.

(A) RNA corresponding to the gene to be evolved (gene L) is transcribed in fusion with an RTtag (region complementary to the RT primer) followed by a region that interacts with the scaffold (in some embodiments SPBM1 being Hfq proximal surface binding module).

(B) Protein corresponding to an RNA binding domain or peptide (RBD) fused to a reverse transcriptase enzyme (RT) via linker peptide (line). The RBD is used to tether RT enzyme to one of the annealing RNAs (in this embodiment, the RT primer).

(C) The transcribed primer RNA consists in a fusion of an RNA sequence motif that is recognized by the RBD (RBM, RNA Binding module), a region that recognizes the scaffold (in some embodiments SPBM2, Hfq distal surface binding module), a region that is the reverse complement of the RTtag (RT primer) and a region that will be released (tRNA in some embodiments) after cleavage by an RNAse (RNAse P in some embodiments).

(D) All molecular elements required for reverse transcription (A, B and processed C) are recruited on the scaffold surface, thus, increasing the likelihood of RNA-dependent DNA polymerization (RdDP).

FIG. 5: Embodiment concerning an improved RT+RH system. The system designed to demonstrate the coupling between RT and RH modules (FIG. 3) was adapted to the improved reverse transcription (FIG. 4). (A) Main modifications include the removal of intron sequence of the KanOn gene, and the design of fusions of KanOn and RT primer to allow recruitment on the scaffold protein in order to improve the likelihood of reverse transcription. Same abbreviations are used.

(B) Detail of the modified plasmid region compared to the system described in FIG. 3C (plasmid VN575).

RBD: RNA binding domain; HPBM: Hfq proximal surface binding module—corresponds to the SPBM2 in the implementation; RBM: RNA binding module recognized by RNA Binding domain (RBD); HDBM: Hfq distal surface binding module—corresponds to the SPBM2 in the implementation.

FIG. 6: Benchmark of different B2H systems tested over a range of affinities from 3 to thousands of nanomolars.

(A) The enhanced B2H system (eB2H, module 3) performs better regarding the direct correlation between affinities and fluorescence signals and the signal/noise ratios. Mean fluorescence intensities (MFI) of peptides with varying affinities (8000, 560, 84 and 3 nM) were evaluated using two-hybrid responsive promoters previously described by Ann Hochchild (dotted line [symbol] ), Rama Ranganathan (dashed line - -♦- -) and, finally, by this work (2 plasmids direct system: - -▴- - VN550+VN515 to VN520; 1 plasmid direct system: - [symbol] - VN750 to VN754 and; 2 plasmids reverse or inverse system: - [symbol] - VN572+VN577 to VN581).

(B) Annotated sequence of the enhanced two-hybrid responsive promoter. OL2-62: lambda phage cI binding site; -35 and -10 boxes for Escherichia coli RNA polymerase sigma factor binding; RBS: ribosome binding site; eGFP: first ATG codon of eGFP is indicated. The predicted transcription start site is indicated.

FIG. 7. Dispersion of enrichment values of silent mutations coding for the wild type protein. Enrichment values were calculated as the ratio of the frequency of a variant after selection by the frequency of the same variant before selection. The data was collected for the interaction between Asf1B variants and IP3. (A) Former version of the B2H corresponding to VN1197 tested in Acella. (B) Current version of the enhanced B2H, corresponding to VN1296 tested in SB33.

FIG. 8: Tunable switch for continuous evolution arrest (module 4) when a strong enough binder variant is produced.

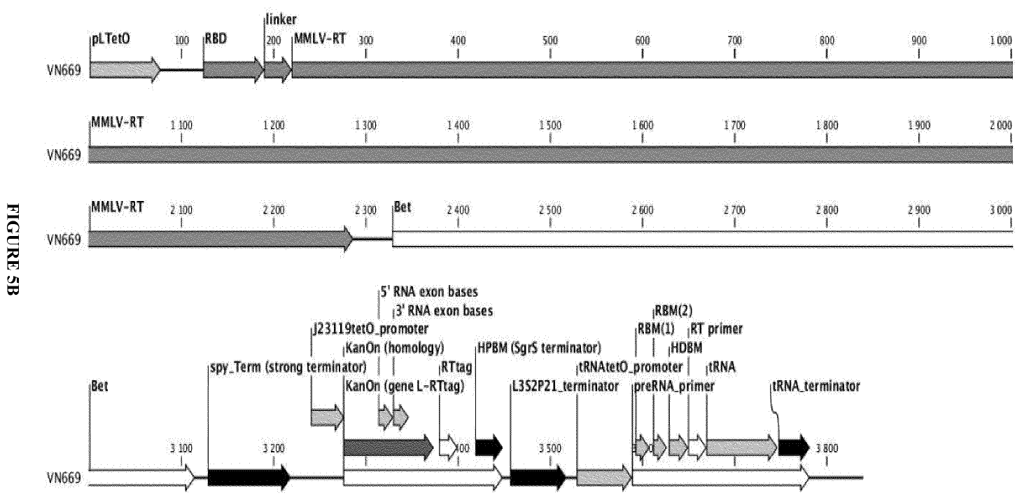

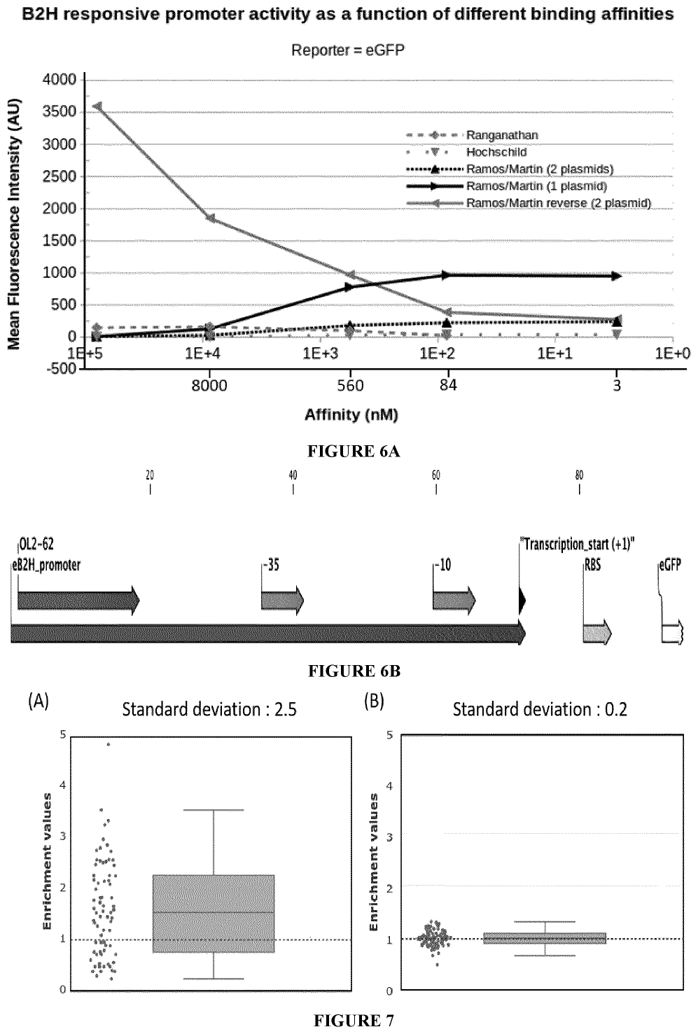

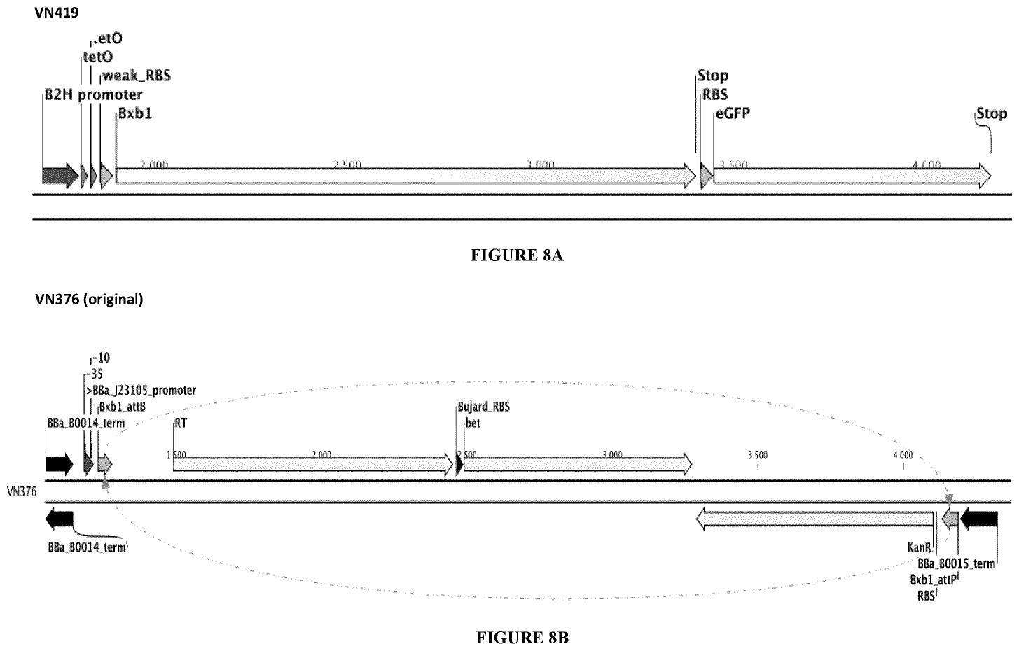

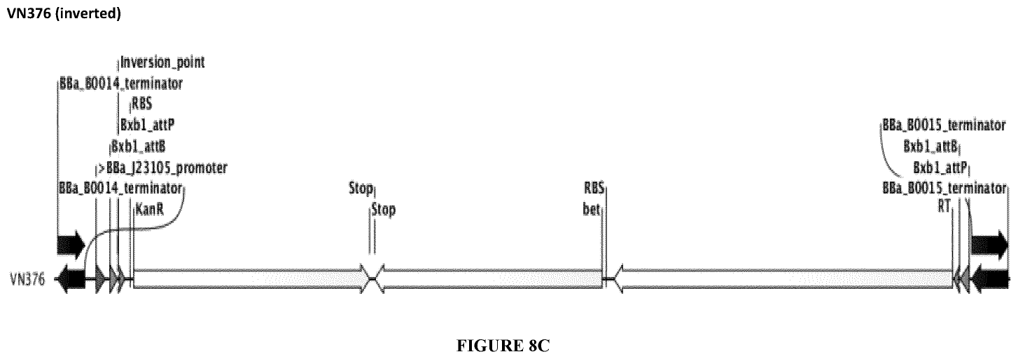

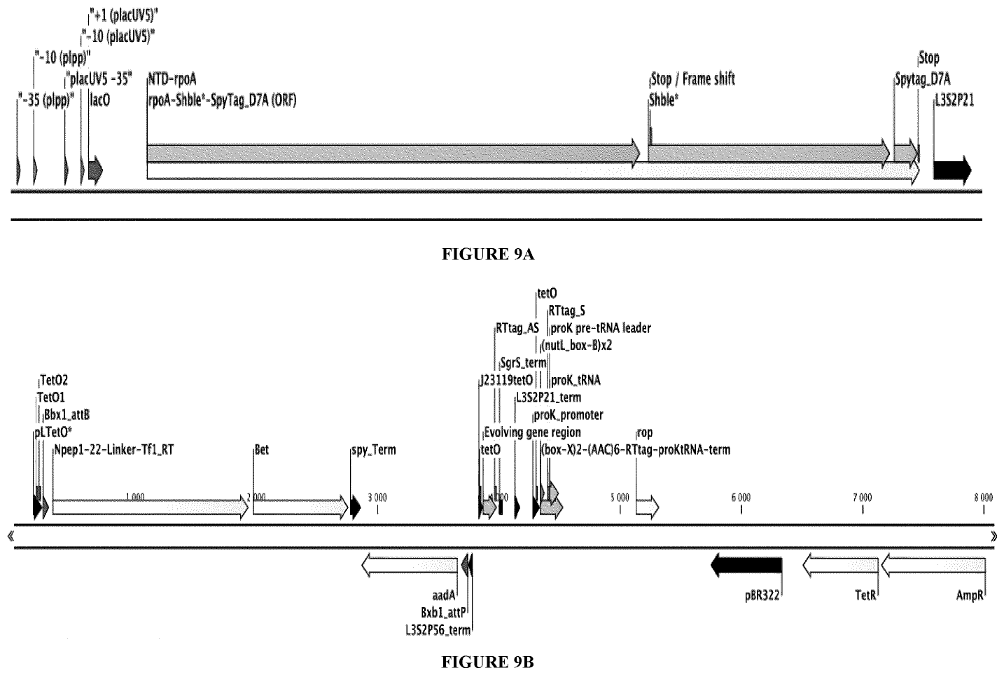

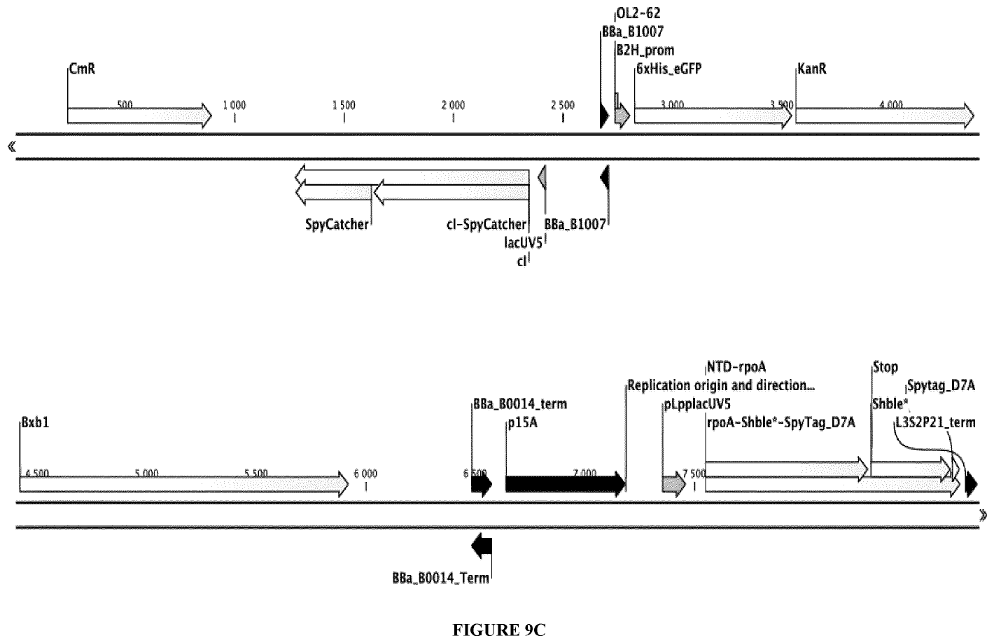

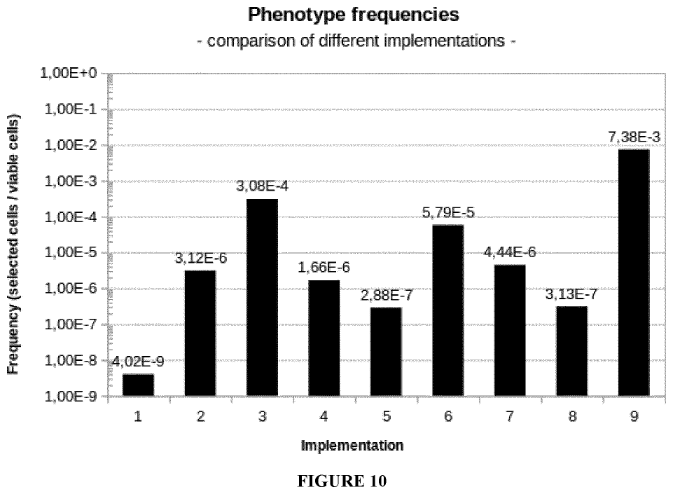

(A) Schematic representation of the B2H responsive cassette constructed in vector VN419. The promoter that triggers the transcription following complex formation (B2H promoter) can be regulated using a repressor protein that can be released from its recognized DNA element (in some embodiments, tetO) using a range of inducer molecule concentration, thereby, tuning the expression of downstream genes and allowing the selection of stronger binders by applying weaker inducer concentrations. If the downstream genes expression exceed a given threshold, the arrest gene (Bbx1) activity will be sufficient to irreversibly block reverse transcription (FIG. 2, module 1) and homologous recombination (FIG. 2, module 2). Consequently, the continuous evolution process stops and a stable binder variant can be identified and characterized for each cell (FIG. 2A).

(B) The genes related to reverse transcription (module 1) and homologous recombination (module 2) can be flanked with DNA sequences (Bxb1 attB and Bxb1 attP) that are recognized by the evolution arrest protein (Bxb1 resolvase/DNA invertase) and consequently their expression can be drastically affected by the latter. In the plasmid VN376, for instance, a bicistronic cassette representing RT gene and 1 bet gene (Bet) are transcribed from a promoter (Bba_J23105 promoter). Downstream, a reporter/marker gene can be coded in the reverse complementary strand (KanR) and is not expressed because it has no associated promoter.

(C) If a strong enough binder is produced, the sense of the genes is inverted (in other words, the DNA fragments between Bxb1_attB and attP sites is inverted) therefore, evolution is stopped and the corresponding cells can be identified and isolated (for instance, in the presence of kanamycin).

FIG. 9: Whole autonomous evolution system implemented in two plasmids.

(A) Zoom in on the ligand hybrid gene comprised in VN1238 plasmid. The gene expression is controlled by a pLPPlacUV5 promoter and a lacO operator (IPTG induced) and codes for a hybrid protein (rpoa-Shble*-SpyTag_D7A) that should be truncated at the N-terminus of Shble domain (Zeocin resistance) because of the presence of a stop codon and a frame shift (Shble*). Only if the stop codon is reverted and the frame shift corrected as expected by the coupling between RT and HR modules the full hybrid construction is expressed (rpoA-Shble-SpyTag_D7A), therefore, the cell become zeocin resistant and fluorescent.

(B) Diversity generation plasmid (VN1228) scheme. The plasmid contains the genetic elements required for generation of diversity including: 1) The gene comprising RT and HR modules. This gene is, respectively, composed by: i) a transcription promoter (pLtetO*) harboring operator regions (TetO) that are recognized by a repressor protein; ii) attB recognition site for an integrase (Bxb1); iii) An open reading frame (ORF) coding for an error-prone reverse transcriptase enzyme (TF1) which N-terminus is fused to an RNA binding domain (RBD, in this implementation corresponds to residues 1-22 of lambda, N-peptide); iv) a ribosome binding site (RBS) that allow the expression of the downstream ORF; v) An ORF that codes for a single-stranded DNA annealing protein (S SAP, lambda bet), vi) a transcription terminator (spy_term); 2) an antibiotic resistance gene (aaDA, streptomycin/spectinomycin resistance) coded in the complementary DNA strand; 3) attP recognition site for an integrase (Bxb1) in the complementary strand; 4) a transcription terminator in the complementary strand (L3S2P56 term), 5) a transcription promoter (J23119tetO) harboring operator regions (TetO) that are recognized by a repressor protein (TetR); 6) the region of the evolving gene that should be diversified which contains in its 3′ region an RTtag_AS (i.e., the reverse complement of an RTtag_S) in order to allow targeted reverse transcription; 7) a transcription terminator that function as Hfq proximal surface binding module (HPBM, SgrS_term—the SPBM1 in this implementation) followed by a spacer and a strong transcription terminator (L3S2P21_term); 8) a transcription promoter (proK_promoter) harboring operator regions (TetO) that are recognized by a repressor protein. The promoter should allow the transcription of an RNA, respectively, composed of by an RNA binding module (RBM) recognized by RBD ((nutL_box-B)×2), an Hfq distal surface binding module (HDBM, (AAC)×6, —the SPBM2 in this implementation), an RTtag_S region, a pre-tRNA (proK tRNA, including its leader sequence in 5′) and a transcription terminator (proK_term); 9) a replication origin (PBR322, rop) and; 10) a bicistronic gene corresponding to an antibiotic resistance gene (AmpR) for selection of transformed cells and a repressor (TetR). The recognition of operator sequences (TetO) on DNA by the repressor (TetR) can be antagonized by an inducer (anhydrotetracycline, aTc), therefore, releasing the transcription from the repressed promoters.

(C) enhanced Bacterial two-hybrid (eB2H) scheme (VN1238). The plasmid contains the elements required for sensing protein-protein interactions inside cells and to arrest the generation of diversity, that is encoded in the first plasmid (VN1228, FIG. 9B), including: 1) an antibiotic resistance gene (CmR, chloramphenicol) for selection of cells transformed by the plasmid; 2) a gene coded in the complementary DNA strand including a promoter (lacUV5), an operator (lacO) recognized by a repressor (lad) and the ORF coding for a hybrid protein (cI-SpyCatcher) corresponding to a DNA binding domain (DBD, cI) and an interaction partner (SpyCatcher); 3) a terminator (bi-directional terminator, Bba_B1007); 4) a gene which expression correlates to the two-hybrid proteins interaction comprising a promoter (B2H_prom), a multicistronic region containing ORFs for reporters, markers and system arrest (fluorescent reporter, eGFP; an antibiotic resistance marker, KanR; and a DNA invertase enzyme, BxB1) and a terminator (bi-directional terminator, Bba_B0014); 5) a replication origin (p15A) and; 6) a gene comprising a promoter (LpplacUV5), an operator sequence (lacO), an ORF coding for a second hybrid protein (rpoA-Shble*-SpyTag_D7A) corresponding to a bacterial RNA polymerase subunit (rpoA) and an interaction partner (Shble*-SpyTag_D7A) and, a terminator (L3S2P21_term).

FIG. 10: Observed frequencies of the expected phenotype for different genetic edition implementations. Different implementation are numbered. (1) Coupling test between RT and HR modules, exclusively, corresponding to the “naive” implementation (without co-localization; plasmids VN575 and VN591). (2) Coupling test between RT and HR modules, exclusively, corresponding to the implementation of the co-localization approach (plasmids VN591 and VN669). (3) Coupling of RT, HR and eB2H modules, exclusively, corresponding to the implementation of co-localization approach and to the selection of edited/fluorescent cells in the presence of zeocin (plasmids VN1228 and VN1237). (4) Same system described in “3” but replacing the bicistronic expression of λ-Bet protein by rhlB (domain that can improve RNA half-life by inhibiting RNAse E, plasmids VN1229 and VN1237). (5) Same system described in “3” but replacing the bicistronic expression of k-Bet protein by Dam (DNA methylase that can improve homologous recombination, plasmids VN1230 and VN1237). (6) Coupling of all modules (RT, HR, eB2H and Stop) corresponding to the implementation with co-localization approach, selection of edited/fluorescent cells in the presence of zeocin and system arrest by DNA inversion (plasmids VN1228 and VN1238). (7) Same system described in “6” but replacing the bicistronic expression of λ-Bet protein by rhlB (VN1229 and VN1238). (8) Same system described in “6” but replacing the bicistronic expression of k-Bet protein by Dam (plasmids VN1230 and VN1238). (9) Same system described in “3” but the frequency of edited cells was estimated by ratio of the number of fluorescent colonies and non-fluorescent colonies in the absence of zeocin.

### DETAILED DESCRIPTION OF THE INVENTION

The present invention relates to methods for generating diversity in a selected gene (gene L) in a bacterial cell, preferably based on an innovative strategy of co-localization.

The strategy of co-localization implies the assembly of a molecular complex in a bacterial cell in order to promote an editing process directed to the gene L. The gene editing process implemented by the methods of the invention is based on the inherent error-rate of any reverse transcriptase (RT), that is responsible for the generation of altered complementary DNA (cDNA) copies from a template RNA comprising the sequence of the gene L. A molecular complex (RTC) may be required for carrying out some methods of the invention and corresponds to the assembly on a scaffold protein (SP), of an RT-containing fusion protein (RBD-RT), a template RNA (tpRNA) comprising the sequence of the gene L and a tag sequence complementary of the primer RNA, and a primer RNA (prRNA) suitable for initiating retro-transcription. According to a preferred aspect of the invention, the RTC assembled on an SP advantageously promotes the reverse transcription of the gene L, thereby enhancing the rate of gene L editing. In particular, the co-localization strategy over an SP developed by the inventors increases the half-life of the involved RNAs, also promotes the double-stranded RNA annealing between the prRNA and tpRNA (i.e., between the tag sequence of tpRNA and the primer sequence required for initiating retro-transcription), and further increases the local concentration of the three partners required for the reverse transcription (RBD-RT, tpRNA and prRNA), which therefore improves the efficiency of cDNA synthesis.

The methods of the invention are particularly useful for evolution purposes in bacteria, and especially, can be used to increase the frequency of occurrence of phenotypes of interest. For instance, the molecular system of the invention can be used for ligand screening or metabolic engineering strategies.

In a first aspect, the invention provides a method for generating diversity in a gene L, using a bacterial cell as a host organism. In a second aspect of the invention, the method is supplemented by the addition of optional effectors that enhance the editing process directed to the gene L. In a third aspect of the invention, the method is adapted and complemented for the specific purpose of ligand screening. In a fourth aspect of the invention, the method adapted for ligand screening is improved to trigger the termination of the gene L editing process when an effective ligand is generated by the method. Further, an additional aspect of the invention relates to DNA vectors comprising all the exogeneous genetic elements required for the implementation of the methods of the invention in a bacterial cell.

In a first aspect of the disclosure, a first module is provided for generating diversity in a gene of interest. In this aspect, the method comprises a step of providing a bacterial cell which comprises an RT protein, a template RNA including a priming sequence and a sequence encoding the gene of interest, and a primer initiating the reverse transcription of the gene of interest by the RT upon the annealing of the priming sequence with the primer. In a specific aspect, the method comprises a step of providing a bacterial cell which comprises the four interacting partners of the RTC, i. e., an RBD-RT fusion protein, a tpRNA, a prRNA and an SP. Accordingly, one of the simplest method of the invention only requires the implementation of the RTC. In addition, as the function of the assembled molecular complex is to synthesize cDNA copies from the tpRNA, the methods of the invention necessarily comprise a second step consisting in placing the bacterial cell in environmental conditions allowing an efficient reverse transcription. These conditions may then vary according to the bacterial species and strain in which the method is applied. Classically, these conditions may correspond to the optimal growth conditions that are known from the person skilled in the art and defined by several environmental factors, such as temperature, nutrients type and levels, aerobic or non-aerobic conditions.

Optionally, the first module for generating diversity can be supplemented by other modular elements expressed by the bacterial cell. In a second aspect of the disclosure, a second module is provided aiming to stably implement mutated cDNA into replicating DNA molecules by the expression of homologous recombination (HR) factors. Functional improvement of the first module can be obtained by protecting the oligonucleotides involved (template RNA and primer RNA, especially tpRNA and prRNA) or generated (cDNA copies) from intracellular degradation, thereby improving cDNA synthesis or stability. These optional elements may be called preservative effectors. For instance, the bacterial cell homeostasis can be modified in order to decrease RNA and/or DNA degradation and the cDNA can be stably implemented into the genome or a plasmid. This stable implementation by the second module can be further improved by impairing the methyl directed mismatch repair (MMR) system function.

In a third aspect of the disclosure, a third module is provided allowing to select a modified ligand for a target molecule. This third aspect of the invention provides methods that are specifically adapted for ligand screening purposes. Such methods imply that the gene L to be edited encodes for a potential ligand. In a first aspect, a potential ligand corresponds to a peptide or a protein that must be mutated in order to be converted in an effective ligand capable of binding to a target molecule. In a second alternative aspect, a potential ligand corresponds to a peptide or a protein that must be modified in order to be converted in an ineffective ligand with impaired binding to a target molecule. The methods for ligand screening according to the third aspect of the invention requires that the bacterial cell further comprises a bacterial double hybrid system (B2H) that expresses both the target molecule and a potential ligand. Alternatively, protein fragment complementation (PCA) can also be used instead of B2H, for instance DHFR complementation or GFP fluorescence complementation). Importantly, the B2H module must be functionally coupled to an HR factor so as to allow the integration of neosynthesized cDNA copies of the gene L in a B2H expression cassette that comprises a copy of the gene L. The additional B2H module then allows to detect binding occurrences between an effective ligand and a given target molecule, via the expression of a reporter into the bacterial cell. According to the design of the B2H elements, the detection of binding occurrence is detected by the reporter signal.

In a fourth aspect of the present disclosure, a fourth module is provided to functionally impair the RT function once an effective ligand has been generated from altered copies of gene L, thereby resulting in the arrest of cDNA synthesis from tpRNA. Therefore, the bacterial cell may further comprise a diversity generation arrest (DGA) module functionally coupled to the B2H system module. According to the design of the DGA module, the HR sequence can also be targeted, resulting in the additional impairment of the HR function.

An additional aspect of the invention relates to DNA vectors that encompass all the exogenous genetic elements required to the implementation of the methods of the invention or to bacterial cells comprising these DNA vectors.

### Definitions

As used herein, a “retro-transcription complex” (RTC) refers to a functional molecular complex comprising a tpRNA, a prRNA, an RBD-RT and an SP, the assembled complex being capable of performing the retro-transcription of the gene L sequence included in the tpRNA.

As used herein, a “template RNA” (tpRNA) refers to an oligoribonucleotide capable of binding to a specific domain of an SP and comprising from 5′ to 3′: a selected gene or gene of interest (gene L); an RTtag sequence operably linked to the gene L coding sequence, the RTtag being substantially complementary to the primer required for initiating the retro-transcription (RTprimer) of the gene L by the RT; and optionally a SPBM1 sequence capable of binding to a specific domain of an SP. According to the disclosure, the template RNA is a transcript of an exogeneous DNA sequence introduced in the bacterial cell. The role of the template RNA in the molecular system is to provide a transcript of the gene L to be retro-transcribed into cDNA copies by the reverse-transcriptase (e.g., RBD-RT).

The “selected gene” or “gene of interest” (gene L) of the tpRNA refers to a sequence of any protein or nucleic acid of interest that should be submitted to the targeted molecular evolution approach of the invention. According to a particular aspect of the disclosure, the gene L codes for a potential ligand whose sequence must be edited by the method of the invention in order to modulate (increase or decrease) its binding to a target molecule. In alternative embodiments, the gene L codes for an enzyme directly or indirectly related to the generation of a molecule of interest.

The “RTtag” of the tpRNA refers to an oligoribonucleotide sequence corresponding to the substantially complementary sequence of another oligoribonucleotide that functions as a primer for reverse transcription (RTprimer). According to the disclosure, the RTtag constitutes the substantially complementary sequence of the RTprimer sequence, thereby allowing a partial double stranded annealing between the prRNA and the tpRNA, more specifically between the RTprimer of the prRNA and the RTtag of the tpRNA, hence enabling the reverse transcription of the gene L by a reverse-transcriptase.

The “Scaffold Protein Binding Module 1” (SPBM1) of the tpRNA refers to an oligoribonucleotide sequence capable of binding to the SP at a specific site (SPS1). In a preferred aspect, the SPBM1 has a secondary structure portion that allows a specific binding to the SP.

As used herein, a “primer RNA” (prRNA) refers to an oligoribonucleotide comprising an RTprimer sequence positioned at the 3′ end, and optionally a SPBM2 sequence capable of binding to a specific domain of an SP and an RT binding module (RBM) sequence capable of binding to the RBD fused to a reverse-transcriptase RT (RBD-RT).

The “RTprimer” of the prRNA refers to an oligoribonucleotide sequence that functions as an efficient primer for the RT, in particular in the context of the RBD-RT fusion protein, thus allowing the initiation of the reverse transcription of the gene L of the tpRNA. According to the disclosure, the RTprimer constitutes the sequence that is substantially complementary to the RTtag sequence, thereby allowing a partial double stranded annealing between the prRNA and the tpRNA, more specifically between the RTprimer of the prRNA and the RTtag of the tpRNA, capable of enabling the reverse transcription of the gene L by a reverse-transcriptase.

The “Scaffold Protein Binding Module 2” (SPBM2) of the prRNA refers to an oligoribonucleotide sequence capable of binding to the SP at a specific site (SPS2). In a preferred aspect, the SPBM2 has a secondary structure portion that allows a specific binding to the scaffold protein SP. Importantly, the SPBM2 of the prRNA sequence is sufficiently distinct from the SPBM1 of the tpRNA as to avoid a binding competition to the same SP binding site, i.e. SPS1 or SPS2.

The “RT binding module” (RBM) of the prRNA refers to an oligoribonucleotide sequence capable of binding to the RBM binding domain (RBD) of the RBD-RT fusion protein. In a preferred aspect, the RBM has a secondary structure portion that is involved in the binding to the RBD of the RBD-RT fusion. This sequence thus allows the prRNA to recruit the RBD-RT in the context of module 1.

As used herein, a “RT-containing fusion protein” (RBD-RT) refers to a fusion protein comprising an RT domain fused to an RBD capable of binding to the prRNA and responsible for the recruitment of the RT fusion protein by the RBM of the prRNA. The RBD of the RBD-RT refers to domain capable of binding to the RBM of the prRNA.

The reverse transcriptase domain (RT), optionally of the RBD-RT, refers to an error-prone RT, i.e. an enzyme capable of generating altered copies of cDNA from an RNA template. Accordingly, the role of the RT used in the methods of the disclosure is to generate altered cDNA copies from the gene L sequence of the tpRNA. Besides, as the error rate of any RT is theoretically >0, it follows that any RT is an error-prone RT and is therefore compatible with the methods of the disclosure. The RT can be a natural or engineered RT.

As used herein, a “scaffold protein” (SP) refers to a protein expressed by the bacterial cell and capable of binding both to the SPBM1 of the tpRNA via a first specific binding site (SPS1) and to the SPBM2 of the prRNA via a second binding site (SPS2). In some aspects, the SP is an endogenous protein constitutively expressed by the bacterial cell. In alternative embodiments, the SP is an exogenous or modified protein expressed by the bacterial cell.

As used herein, a “preservative effector” refers to a protein or peptide that is expressed by the bacterial cell and allows to protect the oligonucleotides from intracellular degradation, in particular the oligoribonucleotides tpRNA and prRNA or the oligodeoxyribonucleotides generated (cDNA copies) by the RT.

As used herein, a single-strand annealing protein (SSAP) intended for “homologous recombination” (HR) refers to a protein capable of exchanging identical or similar DNA sequences from distinct DNA strands. Accordingly, the role of the HR used in the methods of the disclosure is to integrate altered cDNA copies of gene L into DNA vector comprising a copy of the gene L.

As used herein, “MMR” refers to the Methyl Directed Mismatch Repair system. MMR is a highly conserved molecular mechanism that plays an essential role in bacteria by identifying and repairing the DNA mismatch. Classically, mismatch repair occurs on the non-methylated strand of hemi-methylated DNA, which is newly synthesized DNA strand. MMR consists of three important protein components: MutS, MutL, and MutH. MutS is responsible for the recognition of the mismatched base pairs that initiates the mismatch repair; MutL recognizes MutS-DNA heteroduplex complex and the assembly of the MutS-MutL-DNA heteroduplex ternary complex then activates MutH; MutH is responsible for an incision of the neosynthesized unmethylated strand at a hemi-methylated DNA site. According to the methods of the disclosure, MMR system is impaired by certain preservative effectors in order to prevent neosynthetized cDNA strands of the gene L from being removed by the system.

As used herein, the “DNA methylase” (Dam) refers to an enzyme capable of adding methyl groups in neosynthesized DNA. According to the methods of the disclosure, Dam can be expressed or overexpressed in the bacterial cell in order to prevent neosynthesized copies of gene L from being targeted by the MMR system.

As used herein, a “ribonuclease” (RNAse) refers to an enzyme that catalyzes the degradation of RNA strands, such as the RNAse E, the RNAse R or the polynucleotide phosphorylase (PnPase). In bacteria such as Escherichia coli, RNAses are involved in the fast turnover of RNAs that reduces the probability of retro-transcription complex formation, and thus reduce the retro-transcription efficiency of the first module in the context of the disclosure. According to the methods of the disclosure, an RNAse can be mutated in order to impair its degradation function, thereby increasing the RNA stability in the bacterial cell.

As used herein, a “single-strand DNA exonuclease” (ssDNA exonuclease) refers to an enzyme capable of fragmenting ssDNA strands in the bacterial cell by cleaving nucleotides at the 5′ or 3′ end of the ssDNA strand. For instance, xonA, xseA, exoX and recJ are known ssDNA exonucleases. According to the methods of the disclosure, an ssDNA exonuclease can be mutated or invalidated in order to increase the stability of neosynthetized cDNA copies of the gene L.

As used herein, a “bacterial two hybrid” (B2H) system refers to a molecular system designed to detect protein-protein interactions between a ligand (L) and a target molecule (T). The B2H system expresses two fusion proteins, a fusion protein being a potential ligand (FPL) and a fusion protein acting as a receptor (FPR) for the FPL. The B2H system further comprises a DNA sequence, or expression cassette, comprising a reporter gene sequence and a ribosome binding site (RBS), both operably linked to a specific promoter (P). The interest of such a B2H system is to trigger the expression of a reporter protein only when the binding between FPR and FPL occurs.

The “fusion protein Ligand” (FPL) of the B2H system refers to a protein expressed in the bacterial cell that comprises a ligand domain (L), either fused to transcription subunits (e.g., TrSu) capable of recruiting an RNA polymerase or to a DNA binding domain (DBD) capable of binding to a specific DNA site, the other partner, i.e., transcription subunits or DBD, not fused to the ligand domain (L), being fused to a target molecule (T) capable of binding to the ligand (L) domain of the FPL when the L domain correspond to an effective ligand. The L domain of FPL is derived from the expression of a copy of the gene L. The gene L can be both mutated by the RT and integrated into the DNA vector coding the FPL of the B2H system via an HR. As a result, the gene L that encodes the L domain of FPL corresponds to the original version of the gene L or to a modified version of the gene L. Since the L domain of FPL either corresponds to an effective ligand or an ineffective ligand, the L domain of FPL is considered as a potential ligand.

The “fusion protein Receptor” (FPR) of the B2H system refers to a protein expressed in the bacterial cell that comprises a target molecule (T) capable of binding to the ligand (L) domain of the FPL when the L domain correspond to an effective ligand and either a DBD capable of binding to a specific DNA site or transcription subunits (e.g., TrSu) capable of recruiting an RNA polymerase.

The DBD allows the FPR or FPL to bind to a specific DNA site positioned at proximity of the promoter P, so as to promote the recruitment of an RNA polymerase nearby the promoter P when a binding between FPR and FPL occurs, thus allowing the expression of a reporter gene.

As used herein, an “effective ligand” refers to an L domain of FPL capable of binding to the target molecule of FPR, and reciprocally an “ineffective ligand” refers to an L domain that cannot bind to the target molecule. In addition, an “improved ligand” refers to an effective ligand whose binding affinity to the target molecule has been improved compared to those of the original ligand expressed from the original gene L. In contrast, an “debased ligand” refers to an effective ligand whose binding affinity to the target molecule has been decreased compared to those of the original ligand expressed from the original gene L.

As used herein, a “DNA invertase” refers to an enzyme capable of catalysing the inversion of a DNA segment that is flanked by a pair of DNA invertase sites. In a DNA strand, such an inversion results in the replacement of the 5′ end of the targeted sequence by its 3′ complementary end, and vice versa. Accordingly, the role of the DNA invertase used in some methods of the disclosure is to target and invert specific DNA sequences that are flanked by invertase sites. Then, once inverted, the targeted sequence is no longer transcribed as the original DNA sequence but as a completely different sequence. As a result, in case the original DNA sequence codes for a protein, then the inversion by a DNA invertase prevents the expression of this protein.

The term “gene” designates any nucleic acid encoding a protein. The term gene encompasses DNA, such as cDNA or gDNA, as well as RNA. The gene may be first prepared by e.g., recombinant, enzymatic and/or chemical techniques, and subsequently replicated in a host cell or an in vitro system. The gene typically comprises an open reading frame (ORF) encoding a desired protein but could also be reduced to a fragment thereof. The gene may contain additional sequences such as a transcription terminator or a signal peptide.

The term “vector” includes plasmids, cosmids or phages. Preferred vectors are those capable of autonomous replication. In the present specification, “plasmid” and “vector” are used interchangeably, as the plasmid is the most commonly used form of vector. In general, vectors comprise an origin of replication, a multicloning site and a selectable marker.

A nucleic acid is said to be “operably linked” when it is placed into a functional relationship with another nucleic acid sequence. The term “operably linked” means a configuration in which a control sequence is placed at an appropriate position relative to a coding sequence, in such a way that the control sequence directs expression of the coding sequence. In particular, for the purposes of the present invention, a promoter or enhancer is operably linked to a coding sequence if it drives the transcription of the sequence. Generally, “operably linked” means that the DNA sequences being linked are contiguous.

As used herein, an “expression cassette” refers to a construct, whether integrated into a host genome or present on an extra-chromosomal element, which has sufficient elements to permit the expression of the RNA and its translation in a protein when in the proper cell type or under inductive conditions. More particularly, the expression cassette may comprise a promoter (P) capable of recruiting a partner, such as RNA polymerase, that initiates the transcription of the 5′ downstream DNA sequence; an operably linked RBS capable of recruiting ribosomes allowing the translation of the 3′ downstream RNA sequence of the transcribed RNA; an operably linked DNA sequence of interest to be transcribed and translated; and a terminator sequence that causes the arrest of the transcription. According to the disclosure, when a first coding sequence of interest of the expression cassette, e.g., the gene L, is operably linked to the second coding sequence of interest (e.g., TrSu), a protein fusion can be expressed.

As used herein, a “transcription cassette” refers to a construct, whether integrated into a host genome or present on an extra-chromosomal element, which has sufficient elements to permit the expression of the RNA when in the proper cell type or under inductive conditions. More particularly, the expression cassette may comprise a promoter (P) capable of recruiting a partner, such as RNA polymerase, that initiates the transcription of the 5′ downstream DNA sequence; an operably linked DNA sequence of interest to be transcribed; and a terminator sequence that causes the arrest of the transcription.

The term “control sequences” means nucleic acid sequences necessary for expression of a gene. Control sequences may be native, homologous or heterologous. Well-known control sequences and currently used by the person skilled in the art will be preferred. Such control sequences include, but are not limited to, a leader, polyadenylation sequence, propeptide sequence, promoter, signal peptide sequence, and transcription terminator. Preferably, the control sequences include a promoter and a transcription terminator.

The “reporter” of the B2H system refers to a protein expressed by the bacterial cell that generates a signal. The signal can be a luminescence or fluorescence signal. Alternatively, the reporter can be an enzyme producing a product that generates a signal. According to the classical principle of B2H systems, the reporter is expressed when an interaction between two partners, i. e. FPR and FPL in the context of the invention, and the generated signal allows to detect this interaction. For instance, the reporter may be a luminescent or a fluorescent protein such as GFP and its derivatives, in particular the protein eGFP. Alternatively, the signal can also be any antibiotic resistance or any auxotrophic factor.

As used herein, a “promoter” (P) refers to a DNA sequence capable of recruiting an RNA polymerase in order to initiate the transcription of DNA sequences that are operably linked to said promoter, which are positioned downstream in the DNA strand. In addition, according to its sequence, a promoter can strongly promote transcription events (strong promoter) or promote them more moderately (moderate or weak promoter).

As used herein, a “ribosome binding domain” (RBS) refers to an RNA sequence capable of recruiting ribosomes thus allowing the translation of the 3′ downstream RNA sequence. In addition, according to its sequence, an RBS can strongly promote translation events (strong RBS) or promote them more moderately (moderate or weak RBS).

“Heterologous”, as used herein, is understood to mean that a gene or encoding sequence has been introduced into the cell by genetic engineering. It can be present in episomal or chromosomal form. The gene or encoding sequence can originate from a source different from the host cell in which it is introduced. However, it can also come from the same species as the host cell in which it is introduced but it is considered heterologous due to its environment which is not natural. For example, the gene or encoding sequence is referred to as heterologous because it is under the control of a promoter which is not its natural promoter, it is introduced at a location which differs from its natural location. The host cell may contain an endogenous copy of the gene prior to introduction of the heterologous gene or it may not contain an endogenous copy.

As used herein, the term “complementary” refers to complementarity properties of nucleobases that define interactions occurring between specific nucleobases pairs, i.e. between adenine (A)/thymine (T) pairs for DNA, between adenine (A)/uracil (U) pairs for RNA, or between guanine (G)/cytosine (C) pairs for both DNA and RNA molecules. Accordingly, a “complementary pairing” refers to the ability of distinct oligonucleotides, or distinct regions of a single oligonucleotide, to bind each other through a sum of A/T, A/U or G/C pairings. In addition, as used herein the term “substantially complementary” refers to a level of complementarity between two oligonucleotide sequences that is enough to ensure a functional interaction. For instance, the nucleotides are complementary at 70, 75, 80, 85, 90, 95, 99 or 100% when two sequences are substantially complementary. Optionally, 1, 2 or 3 mismatches can be present when two sequences are substantially complementary.

The term “recombinant bacterium”, “recombinant bacterial cell”, “genetically modified bacterium” or “genetically modified bacterial cell” designates a bacterium that is not found in nature and which contains a modified genome as a result of either a deletion, insertion or modification of genetic elements or which contains a vector or a set of vectors. A “recombinant nucleic acid” therefore designates a nucleic acid which has been engineered and is not found as such in wild type bacteria.

The term “about” means more or less 5% of a number. For instance, about 100 means between 95 and 105.

### Module 1: Diversity Generation

The first module comprises means for allowing to generate diversity from a gene of interest in a bacterial cell.

By “gene” is intended to refer to any nucleic acid of interest, not only nucleic acid of interest encoded by a gene. The gene of interest may code for a protein, a nucleic acid (DNA or RNA) or enzymes (protein, DNA or RNA based) such as an antisense nucleotide, DNAzyme, ribozyme, DNA modifying enzymes, RNA modifying enzymes, metabolic enzymes and pathways, RBSs, DNA binding proteins, RNA binding proteins, RNA motifs recognized by proteins, RNA/RNA interaction modules and partners of protein complexes. Roughly, every nucleotide sequence that can be transcribed, retrotranscribed and can be used as substrate for HR can potentially be diversified and evolved in DNA, RNA and protein levels. In a particular aspect, the gene of interest encodes a binding partner of a complex comprising at least a ligand molecule and a target molecule. Optionally, the gene of interest is intronless.

The diversity is created by a reverse-transcription by a reverse transcriptase RT of an RNA comprising the gene of interest, leading to the production of error-prone generation of cDNA in a bacterial cell. Indeed, the RT is responsible for the retro-transcription of the gene L of the tpRNA, thereby generating diversity with neosynthesized altered copies of the gene L. This generation of diversity thus allows the emergence of new variants from gene L, i. e. new nucleic acid sequences or new protein variants. These new variants may reveal new biological properties including properties of interest. The RT, optionally of the RBD-RT, is a low-fidelity RT and/or an RT with a high initiation rate/processivity. A low-fidelity RT is characterized by a relatively high error rate that favors the synthesis of altered cDNA copies from gene L, i.e. an error rate ranging from about 10⁻⁶ to about 10⁻⁴, preferably from about 10⁻⁵ to about 10⁻⁴ error per nucleotides and more preferably an error rate of about 10⁻⁴ error per nucleotides. In addition, a high initiation rate/processivity RT increases the number of retro-transcriptions performed for a single enzyme. The RT can be an engineered RT from any source.

In a more preferred aspect, the RT is a low fidelity RT from sources such as retroviruses, transposons, retrons or diversity generating elements. RTs are well-known to the person skilled in the art and some RTs are disclosed for instance in Jamburuthugoda et al (J Mol Biol. 2011, 407(5):661-72), Menendez-Arias et al (Viruses. 2009, 1(3):1137-65) or Kirshenboim et al (Virology. 2007, 366(2):263-76). In even more preferred aspect, the RT is selected in the group consisting in: the RT of the Long Terminal Repeat (LTR) retrotransposon Tfl, the human immunodeficiency virus type 1 (HIV-1) RT, the simian immunodeficiency virus (SIV) RT, the feline immunodeficiency virus (FIV) RT, the Moloney murine leukemia virus (MMLV) RT (SEQ ID NO: 3), the feline leukemia virus (FeLV) RT, the alfalfa mosaic virus (AMV) RT, or the prototype foamy virus (PFV) RT.

In a particular aspect, the RT sequence is the sequence of the Tfl RT corresponding to SEQ ID NO: 1. In an alternative particular aspect, the RT sequence is the sequence of the HIV-1 RT corresponding to SEQ ID NO: 2 and SEQ ID NO: 57. In another alternative particular aspect, the RT sequence is the sequence of the MMLV RT corresponding to SEQ ID NO: 3.

Optionally, the RT is fused with a domain binding the prRNA (RBD). The RT can be fused either at its N terminal end or at its C terminal end with the binding domain (RBD), optionally through a linker. As used herein, the term “linker” refers to a sequence of at least one amino acid that links the RT and the RBD. Such a linker may be useful to prevent steric hindrances. The linker is usually 3-44 amino acid residues in length. Preferably, the linker has 3-30 amino acid residues. In some embodiments, the linker has 3, 4, 5, 6, 7, 8, 9, 10, 11, 12, 13, 14, 15, 16, 17, 18, 19, 20, 21, 22, 23, 24, 25, 26, 27, 28, 29 or 30 amino acid residues. Example of linker sequences are Gly/Ser linkers of different length including (Gly4Ser)4, (Gly4Ser)3, (Gly4Ser)2, Gly4Ser, Gly3Ser, Gly3, Gly2ser and (Gly3Ser2)3, in particular (Gly4Ser)3.

In a preferred aspect, the prRNA further comprises a transfer RNA (tRNA) sequence contiguously positioned downstream of the RTprimer sequence. The optional tRNA sequence comprises a specific site between the RTprimer and the tRNA that can be cleaved by a RNAse expressed in the bacterial cell, thereby producing a well-defined 3′ end of prRNA corresponding to the RTprimer and a tRNA. Using tRNA specific sites that can be cleaved off from prRNA allows to yield a free 3′-OH required at the RTprimer for retro-transcription, thereby enhancing the efficacy of the module 1. For instance, the specific site of the optional tRNA sequence is cleaved by a RNAse P expressed by the bacterial cell. Any tRNA sequence could be implemented here and for instance the tRNA sequence corresponds to SEQ ID NO:4.

In order to improve the generation of diversity, a strategy of co-localization of RT, prRNA and tpRNA forming the RTC has been developed. This strategy is based on the binding of these three elements on a scaffold protein SP. Indeed, the co-localization strategy significantly enhances the retro-transcription rate and thereby leads to an enhanced frequency of occurrence of new variants from gene L. For instance, prRNA and tpRNA each comprise a sequence capable of binding the SP while the RT is fused to a domain capable to bind the prRNA or the tpRNA, preferably the prRNA.

According to this preferred aspect, the tpRNA and prRNA respectively comprise SPBM1 and SPBM2 sequence, prRNA further comprises an RBM sequence and the RT is fused with a domain binding RBM (RBD) into an RBD-RT fusion protein.

Number of pairs of peptide-RNA have been disclosed in the art (Keryer-Bibens et al, 2008, Biol. Cell., 100, 125-38; Lunde et al, 2007, Nat Rev Mol Cell Biol, 8, 479-90; Fujimori et al, 2012, Bioinformation, 8, 729-30; Cook et al, 2011, Nucleic Acids Res, 39, D301-8; Chao et al, 2008, Nat Struct Mol Biol, 15, 103-5; Delebecque et al, 2012, Nat Protoc, 7, 1797-807; Kappel et al, 2019, Proc Natl Acad Sci USA, 116, 8336-8341; Kappel et al, 2019, 27, 140-151, the disclosure thereof being incorporated herein by reference; DataBases (rbpdb.ccbr.utoronto.ca and pri.hgc.jp). Based on this knowledge, the person skilled in the art is able to design this co-localization (tethering) elements, in particular the SP, SPBM1 and SPBM2 on one side and RBM and RBD on the other side.

In a particular aspect, the RBM of the prRNA comprises a secondary structure, preferably a stem-and-loop RNA secondary structure, wherein the stem consists in 10 to 20 paired complementary nucleotides and the loop is composed of 4 to 6 unpaired nucleotides. Also, the stem can comprise one unpaired nucleotide that breaks the homogeneity of nucleotides pairing into the stem portion. In a particular aspect, the sequence of the RBM of the prRNA corresponds to Lambda BoxB from nutL (SEQ ID NO:7) and the associated RBD of RBD-RT corresponds to the Lambda phage N protein sequence (SEQ ID NO:5, SEQ ID NO:6). In an alternative particular aspect, the sequence of the RBM of the prRNA corresponds to a wild type MS2 binding motif (SEQ ID NO:9) or to a high affinity variant of the MS2 binding motif (SEQ ID NO:10) and the associated RBD of RBD-RT corresponds to the MS2 phage coat protein sequence (SEQ ID NO:8). In another alternative aspect, the sequence of the RBM of the prRNA corresponds to the PP7 binding motif (SEQ ID NO:12) and the associated RBD of RBD-RT corresponds to the PP7 phage coat protein sequence (SEQ ID NO:11).

Optionally, the RBM may bind to the RBD with a relatively high affinity, i.e. an affinity characterized by a dissociation constant (Kd) lower than 1·10⁻⁷M, preferably between 1·10⁻⁸ and 1·10⁻⁹M.

In a preferred aspect, the SPBM1 and the SPBM2 have at least a secondary structure portion that is involved in a specific binding to the SP, respectively to SPS1 and SPS2.

Optionally, the SPBM1 and/or SPBM2 may bind to the SP with a relatively high affinity, i.e. an affinity characterized by a dissociation constant (Kd) lower than 1·10⁻⁷M, preferably between 1·10⁻⁸ and 1·10⁻⁹M.

The RTprimer and RTtag sequences are selected in order to have complementary sequences and to be suitable for initiating reverse-transcription by the RT, especially RBD-RT, of the gene L. In a particular aspect, the sequence of RTprimer corresponds to SEQ ID NO:13 and the sequence of RTtag corresponds to SEQ ID NO:14.

In a particular aspect, the SP is the Host factor required for replication of the RNA phage Qβ (Hfq) protein or a fragment or variant thereof. Any bacterial Hfq is suitable. Preferably, the Hfq endogenous of the bacterial cell can be used. Alternatively, the Hfq is from another bacteria. In a particular embodiment, the Hfq is from Escherichia coli. According to this particular aspect, the sequence of the SP can correspond to SEQ ID NO:15. The Hfq presents an advantageous quaternary arrangement that allows multiple binding sites to RNA motifs such as SPBM1 and SPBM2. In addition, the native Hfq protein comprises binding sites that allow interactions with the RNAse E, a relatively well-conserved RNAse in bacteria that is capable of cleaving RNA such as tpRNA and prRNA partners. To avoid disadvantageous cleavages and thus favor RNA stability, the Hfq may be modified with a C-terminus deletion (HfqΔC-term) in order to hamper its membrane localization in proximity to RNAse E. Accordingly, in a more preferred aspect, the SP is a modified HfqΔC-term and that allows an advantageous reduction of the interactions between RNAse E and the SP. As disclosed in Vecerek et al (Nucleic Acids Research, 2008, 36, 133-143), the essential part of Hfq, e.g. from E coli, for the hexamer core is the 65 N terminal residues of the protein. Therefore, the fragment of Hfq preferably comprises fragment corresponding to the residues 7-65 of SEQ ID NO: 15. Several HfqΔC-term variants have been disclosed such as Hfq 83 (with deletion of residues 84-102), and Hfq 65 (with deletion of residues 66-102). According to this alternative aspect, the sequence of the modified SP can correspond to SEQ ID NO:16. Alternatively, the SP can be modular and can be a fusion protein of different RNA binding protein, such as different phage coat proteins, for instance a fusion protein of MS2 phage coat protein and PP7 phage coat protein. Accordingly, the SPBM1 and SPBM2 could be the MS2 binding motif and the PP7 binding motif.

In a specific aspect, the SP is Hfq, a variant or a fragment thereof. In this specific aspect, SPBM1 and/or SPBM2 can be selected in the group consisting of SEQ ID NOs: 17 or 18. In a very particular aspect, SPBM1 has the sequence of SEQ ID NO: 17 and SPBM2 has the sequence of SEQ ID NO: 18.

In some aspects, the tpRNA further comprises a linker or spacer domain of variable size that is positioned between the RTtag sequence and the SPBM1 sequence. In other aspects, the prRNA further comprises a linker or spacer domain of variable size that is positioned between the RTprimer sequence and the SPBM2 sequence, the RTprimer sequence and the RBM sequence and/or the SPBM2 sequence and the RBM sequence. In addition, theses domains may adjust the relative positioning of the three partners involved in the reverse transcription, namely tpRNA, prRNA and RBD-RT, in order to enhance the retro-transcription rate of the module 1.

In a specific aspect, the prRNA comprises from 3′ end to 5′, the RTprimer sequence positioned in 3′ end of the prRNA, the SPBM2 and the RBM. Alternatively, the prRNA may comprise from 3′ end to 5′, the RTprimer sequence positioned in 3′ end of the prRNA, the RBM and the SPBM2. During the design of the prRNA and tpRNA, the RNA secondary structure can be checked, for instance by available software allowing to predict the RNA secondary structure, in order to avoid disturbing the secondary structures, in particular of SPBM1, SPBM2 or RBM.

In a very specific aspect, the SP is a Hfq protein, in particular the Hfq of SEQ ID NO: 15, a variant or a fragment thereof; the tpRNA comprises from 5′ to 3′: the gene L or an insertion site suitable for introducing the gene L, an RTtag sequence, preferably of SEQ ID NO: 14, operably linked to the gene L and the SPBM1 of SEQ ID NO: 17; the prRNA comprises from 3′ to 5′: an RTprimer sequence positioned in 3′ end of the prRNA, preferably of SEQ ID NO: 13, the SPBM2 of SEQ ID NO: 18 and the RBM of SEQ ID NO: 7; and the RBD-RT comprises an RT, especially TF1 RT (e.g., of SEQ ID NO: 1), MMLV RT (SEQ ID NO: 3) or HIV-1 RT (e.g., of SEQ ID NO: 2 or 57), fused to an RBD of SEQ ID NO: 5. If the RT is from HIV, One of the subunit is fused to the RBD and the other subunit is co-expressed. In a particular aspect, the fused subunit is p66 (SEQ ID NO: 2). In another particular aspect, the fused subunit is p51 (SEQ ID NO: 57).

The present invention relates to a bacterial cell comprising SP, tpRNA, prRNA and RBD-RT as detailed above in any aspect and the use thereof for generating diversity in a gene of interest.

The present invention relates to a method for generating diversity in a gene L, comprising:
providing a bacterial cell comprising a molecular complex formed by the association of:
   a tpRNA comprising from 5′ to 3′: the gene L, an RTtag sequence operably linked to the gene L;
   a prRNA comprising: an RTprimer sequence positioned in 3′ end of the prRNA,
   a reverse transcriptase (RT), especially TF1 RT (e.g., of SEQ ID NO: 1), MMLV RT (e.g., SEQ ID NO: 3) or HIV-1 RT (e.g., of SEQ ID NO: 2 and 57); and
placing the bacterial cell in conditions that allow the reverse transcription of the gene L, thereby generating altered copies of said gene L of the tpRNA.

Preferably, the present invention relates to a method for generating diversity in a gene L, comprising:
providing a bacterial cell comprising a molecular complex formed by the association of:
   an SP, preferably Hfq or a variant or fragment thereof, optionally a Hfq of Escherichia coli such as the Hfq of SEQ ID NO: 15;
   a tpRNA comprising from 5′ to 3′: the gene L, an RTtag sequence, preferably an RTtag of SEQ ID NO: 14, operably linked to the gene L and a SPBM1 sequence capable of binding to the SP, preferably a SPBM1 of SEQ ID NO: 17;
   a prRNA comprising: an RTprimer sequence positioned in 3′ end of the prRNA and capable of complementary pairing to the RTtag sequence, preferably an RTprimer of SEQ ID NO: 13, a SPBM2 sequence capable of binding to the SP, preferably the SPBM2 of SEQ ID NO: 18, and an RBM, preferably the RBM of SEQ ID NO:7,
   a fusion protein (RBD-RT) comprising a reverse transcriptase (RT) and an RBD capable of binding to the RBM of the prRNA, preferably an RT, especially TF1 RT (e.g., of SEQ ID NO: 1), MMLV RT (e.g., SEQ ID NO: 3) or HIV-1 RT (e.g., of SEQ ID NO: 2 or 57), fused to an RBD of SEQ ID NO: 5; and
placing the bacterial cell in conditions that allow the reverse transcription of the gene L, thereby generating altered copies of said gene L of the tpRNA.

The present invention further relates to a vector or set of vectors, said vector or set of vectors comprising:
a transcription cassette (tC1) comprising a sequence encoding a pre-tpRNA operably linked to a promoter (P1), said pre-tpRNA comprising from 5′ to 3′: an insertion site suitable for the insertion of a gene L, an RTtag sequence, preferably an RTtag of SEQ ID NO: 14, operably linked to the gene L to be inserted and a SPBM1 sequence, preferably a SPBM1 of SEQ ID NO: 17, wherein said tC1 is suitable for allowing, in the bacterial cell, the transcription of a tpRNA including an inserted gene L, wherein the SPBM1 is capable of binding to an SP present in the bacterial cell at a first specific binding site (SPS1);
a transcription cassette (tC2) comprising a sequence encoding a prRNA operably linked to a promoter (P2), said prRNA comprising: an RBM sequence positioned in 5′ end, preferably the RBM of SEQ ID NO: 7, an SPBM2 sequence, preferably the SPBM2 of SEQ ID NO: 18, and an RTprimer, preferably an RTprimer of SEQ ID NO: 13, wherein said tC2 is suitable for allowing, in the bacterial cell, the transcription of a prRNA, wherein the RTprimer is capable of complementary pairing to the RTtag, the SPBM2 is capable of binding to the SP, the sequence encoding the prRNA optionally further comprising a sequence encoding a tRNA sequence contiguously positioned downstream of the RTprimer sequence, a site cleavable by an RNAse of the bacterial cell is present between said tRNA sequence and said RTprimer, thereby allowing the production of a well-defined 3′ prRNA end;
an expression cassette (eC1) comprising a sequence encoding an RBD-RT fusion protein operably linked to a promoter (P3), said RBD-RT comprising a reverse transcriptase (RT) sequence, especially TF1 RT (e.g., of SEQ ID NO: 1), MMLV RT (e.g., SEQ ID NO: 3) or HIV-1 RT (e.g., of SEQ ID NO: 2), and an RBD sequence, preferably an RBD of SEQ ID NO: 5, wherein said eC1 is suitable for allowing, in the bacterial cell, the expression of the RBD-RT fusion protein, wherein the RBD is capable of binding to the RBM of prRNA, and
optionally, an expression cassette (eC2) comprising a sequence encoding the SP operably linked to a promoter (P4), preferably said SP being the Hfq protein, preferably the Hfq of SEQ ID NO: 15, wherein eC2 is suitable for allowing, in the bacterial cell, the expression of the SP, preferably the Hfq protein.

The present invention also relates to a vector or set of vectors comprising the elements as defined below and a bacterial cell comprising this vector or set of vectors or comprising the elements as defined below, the elements being:
a transcription cassette (tC1) comprising a sequence encoding a tpRNA operably linked to a promoter (P1), said tpRNA comprising from 5′ to 3′: a gene L, an RTtag sequence operably linked to the gene L and a SPBM1 sequence, wherein said tC1 is suitable for allowing, in the bacterial cell, the transcription of a tpRNA, wherein the SPBM1 is capable of binding to an SP present in the bacterial cell at a first specific binding site (SPS1);
a transcription cassette (tC2) comprising a sequence encoding a prRNA operably linked to a promoter (P2), said prRNA comprising: an RBM sequence positioned in 5′ end, preferably the RBM of SEQ ID NO: 7, an SPBM2 sequence, preferably the SPBM2 of SEQ ID NO: 18, and an RTprimer, preferably an RTprimer of SEQ ID NO: 13, wherein said tC2 is suitable for allowing, in the bacterial cell, the transcription of a prRNA, wherein the RTprimer is capable of complementary pairing to the RTtag, the SPBM2 is capable of binding to the SP, the sequence encoding the prRNA optionally further comprising a sequence encoding a tRNA sequence contiguously positioned downstream of the RTprimer sequence, a site cleavable by an RNAse of the bacterial cell is present between said tRNA sequence and said RTprimer, thereby allowing the production of a well-defined 3′ prRNA end;
an expression cassette (eC1) comprising a sequence encoding an RBD-RT fusion protein operably linked to a promoter (P3), said RBD-RT comprising a reverse transcriptase (RT) sequence, especially TF1 RT (e.g., of SEQ ID NO: 1), MMLV RT (e.g., SEQ ID NO: 3) or HIV-1 RT (e.g., of SEQ ID NO: 2), and an RBD sequence, preferably an RBD of SEQ ID NO: 5, wherein said eC1 is suitable for allowing, in the bacterial cell, the expression of the RBD-RT fusion protein, wherein the RBD is capable of binding to the RBM of prRNA, and
an expression cassette (eC2) comprising a sequence encoding the SP operably linked to a promoter (P4), preferably said SP being the Hfq protein, preferably the Hfq of SEQ ID NO: 15, wherein eC2 is suitable for allowing, in the bacterial cell, the expression of the SP, preferably the Hfq protein.

Preferably, the vector or the set of vectors is low copy vectors.

In a particular aspect, the diversity generation could be multiplexed in order to allow the co-evolution of several genes of interest, allowing for instance the evolution of biological pathways or multiprotein complexes. In the context of a multiplexed method, then a couple of tpRNA and prRNA will be designed for each gene of interest to be evolved. For instance, for a pathway or complex comprising two genes of interest, the method comprises the providing of a first couple of tpRNA and prRNA for the first gene of interest and of a second couple of tpRNA and prRNA for the second gene of interest. If the module 1 is carried out with an SP, the same system of SP, SPBM1 and 2, SPS1 and 2, RBM and RBD can used for the different couples of tpRNA and prRNA or distinct systems can be used for each couple of tpRNA and prRNA. Alternatively, different tpRNAs with the same RTtag could share the same prRNA. The multiplexed version of the invention can be applied, for instance, for metabolic engineering or strain development.

It is believed that it is the first time that the use of an error-prone retroviral/retrotransposon reverse transcriptase in bacteria for evolution purposes is reported, as well as the strategy of using pre-tRNA fusions to obtain RNAs with well defined 3′ sequence that are required for efficient reverse transcription. Indeed, the inventors overcome a series of difficulties such as the very short half-life of RNAs and linear DNA in bacteria that result, respectively, in low reverse transcription efficiency and low cDNA amounts, in particular by the combination of the module 1 with the module 2.

### Module 2: Preservative Effectors

The second module comprises means for allowing to improve the stability of oligonucleotides in the bacterial cell.

The second module is an optional module that can be combined to the first module in order to enhance the retro-transcription efficiency of the RT.

In a preferred second aspect, the preservative effector corresponds to an HR factor that is expressed or overexpressed by the bacterial cell. Advantageously, the HR factor of the second module can integrate the neosynthesized cDNA copies of gene L in DNA vectors that comprises a copy of the gene L. Such an integration thus prevents neosynthesized cDNA copies from degradation in the bacterial cell. Accordingly, the HR factor allows to replace a copy of the gene L included in a vector introduced into the bacterial cell or a copy of the gene L present in the genome of the bacterial cell, e.g., vector(s) that encodes exogenous required elements of the modules, described herein. Importantly, the capacity of HR factor to integrate the cDNA copies of gene L generated by the module 1 into a DNA vector or set of vectors that codes for elements of the module 3, allows functional coupling between the first and third modules.

The HR factor is a recombinase that mediates recombination-mediated genetic engineering using single-strand DNA, in particular the neosynthesized cDNA copies of the gene L. The HR factor is preferably a beta recombinase. Beta recombinase binds to ssDNA and anneals to the ssDNA to complementary ssDNA such as, for example, complementary genomic DNA. The beta recombinase can be a recombinase as disclosed in Datta et al (Proc Natl Acad Sci USA 105: 1626-1631 (2008)) or a recombinase selected in the non-exhaustive group comprising bet of lambda phage of E coli, s065/s066 of SXT element of Vibrio cholerae, plu2935 of Photorhabdus luminescens, EF2132 of Enterococcus faecalis, recT of Rac prophage of E coli, orfC of Legionella pneumophila, gp35 of SPP1 phage of Bacillus subtilis, gp61 of Che9c phage of Mycobacterium smegmatis, orf48 of A118 phage of Listeria monocytogenes, orf245 of ul36.2 of Lactococcus lactis or gp20 of phiNM3 phage of Staphylococcus aureus. See also, recombinase as disclosed in WO2017/184227, the disclosure thereof being incorporated herein by reference.

In a more preferred aspect, the HR factor of the second module corresponds to a beta recombinase such as the lambda phage recombinant factor (λBet) whose sequence may correspond to SEQ ID NO: 19.

If the method includes the modules 3 and 4, then the RH factor is mandatory. Of course, in order to obtain the recombination, the bacterial cell comprise a copy of the gene L or a part thereof suitable for allowing the introduction of a neosynthesized copy of the gene L into the vector or genome by recombination. In a preferred aspect, the copy of the gene L or a part thereof is operably linked to a promoter, more preferably part of an expression cassette. The expression cassette may further comprise elements of module 3.

The present invention relates to a bacterial cell comprising the above-mentioned components of the first module, preferably the tpRNA, the prRNA and RT, more preferably the SP, the tpRNA, the prRNA and the RBD-RT, and further comprises an HR factor, preferable beta recombinase such as Bet and the use thereof for generating diversity in a gene of interest and for increasing the stability of oligonucleotides in the bacterial cell, thereby improving the generation of diversity in a gene L.

The present invention relates to a method for generating diversity in a gene L comprising any aspect of the two steps described for the module 1, wherein the bacterial cell further comprises an HR factor, preferable beta recombinase such as λBet.

The present invention further relates to a vector or set of vectors as described for the module 1, i.e. comprising tC1, tC2, eC1 and optionally eC2, and further comprising:
an expression cassette (eC3) comprising an HR factor gene operably linked to a promoter (P5), wherein said eC3 is suitable for allowing, in the bacterial cell, the expression of an HR factor capable of integrating the altered copies of the gene L into a DNA vector or into the genome of the bacterial cell, said vector or genome comprising a copy of the gene L, thereby preserving the altered copies of the gene L from degradation.

The present invention also relates to a vector or set of vectors as described for module 1 that further comprises the elements described below, and a bacterial cell comprising this vector or set of vectors or comprising the elements of the vector or set of vectors as described for module 1 and elements as defined below, the elements being:
an expression cassette (eC3) comprising an HR factor gene operably linked to a promoter (P5), wherein said eC3 is suitable for allowing, in the bacterial cell, the expression of an HR factor capable of integrating the altered copies of the gene L into a DNA vector or into the genome of the bacterial cell, said vector or genome comprising a copy of the gene L, thereby preserving the altered copies of the gene L from degradation.

Preferably, the HR factor gene is a beta recombinase, especially λBet.

Preferably the vector or the set of vectors is low copy vector.

### Module 3: Two Hybrid System (B2H)

The module 3 can be added to the modules 1 and 2. This module is a bacterial two-hybrid system suitable for selecting variants of the gene L based on their binding capacity to a target molecule (T). In particular, the functional coupling between the first module and the third module requires the presence of a second module that necessarily comprises an HR factor. Alternatively, the module 3 in its improved and optimal aspects is also of interest even in absence of the modules 1 and 2 as further discussed below.

Importantly, the addition of the third module allows to adapt the methods disclosed herein for ligand screening purposes. Indeed, the third functional module comprises a B2H system whose components are expressed by the bacterial cell in order to detect interactions between FPR (a fusion protein comprising the target molecule) and FPL (a fusion protein comprising the ligand domain encoded by the variants of the gene L, generated by the diversity generation of module 1 and integrated into a vector/genome by the homologous recombination of module 2).

According to the third aspect of the disclosure, the FPL comprises a ligand domain that is derived from a copy of the gene L that is included in a DNA vector of the bacterial cell. Since the required HR allows to integrate altered copies of the gene L in such a vector, the L domain of the FPL can be modified and ligand variants can thus be generated. Modifications of the original gene L coding ligand domain of FPL can convert an original ineffective ligand domain into an effective ligand domain. Conversely, an original effective ligand can be converted in an improved, debased or ineffective ligand domain.

Different ligand screening strategies can be implemented. In case the original gene L encodes an ineffective ligand, some methods according to the third aspect of the disclosure allow to detect altered copies of the gene L that are responsible for the expression of an effective ligand. Alternatively, in case the original gene L encodes an effective ligand, methods according to the third aspect of the disclosure allow to detect altered copies of the gene L that are responsible for the expression of an improved, debased or ineffective ligand.

The B2H system of the third functional module allows to positively couple the binding events between FPR and FPL with the expression of the reporter gene.

For instance, when the L domain of FPL corresponds to an effective ligand, the interaction between FPL and FPR allow to recruit an RNA polymerase that interacts with a promoter operably linked to the reporter gene, so as to trigger the expression of the latter. The signal intensity provided by the reporter protein is thus directly correlated to the binding affinity of the ligand. In a consistent manner, when an effective ligand is converted in an improved ligand, the quantifiable reporter signal increases. Conversely, when an effective original ligand is converted in an ineffective ligand, the quantifiable reporter signal decreases.

The quantification of the reporter signal is particularly important in ligand screening methods, since it allows to select a desired ligand variant, i.e. an effective, improved, debased or ineffective one, encoded by an altered copy of the gene L. More particularly, ligand screening methods implementing the third module of the disclosure allow the selection of the ligand variant encoded by an altered copy of the gene L when the reporter is expressed, optionally at least at a predetermined level.

In an alternative aspect, the B2H system of the third module allows to negatively couple the binding events between FPR and FPL with the expression of the reporter gene. Then, the present disclosure relates to a method for screening a ligand molecule capable of binding a target molecule from variants encoded by altered copies of a gene L, wherein the bacterial cell comprises a bacterial two-hybrid system (B2H) comprising a construct with a promoter (P), a sequence defining a ribosome binding site (RBS) and a reporter gene, the P sequence being operably linked to the RBS sequence and the reporter gene, and the expression of the promoter being controlled the B2H system including FPR and FPL, and the method comprises the selection of the variant encoded by an altered copy of the gene L when the reporter is expressed, optionally at least at a predetermined level.

In this aspect, when the L domain of FPL corresponds to an effective ligand, the interaction between FPL and FPR allow to recruit an RNA polymerase that interacts with a promoter operably linked to a repressor gene. The B2H-regulated repressor gene then allows to inhibit the transcription from the promoter gene operably linked to the reporter gene, thereby decreasing the expression of said reporter gene. The signal intensity provided by the reporter protein is thus indirectly correlated to the binding affinity of the ligand. Therefore, when an effective ligand is converted in an improved ligand, the quantifiable reporter signal decreases or disappears.

Conversely, when an effective original ligand is converted in an altered or ineffective ligand, the quantifiable reporter signal increases.

Then, the present disclosure relates to a method for screening a ligand molecule capable of binding a target molecule from variants encoded by altered copies of a gene L, wherein the bacterial cell comprises a bacterial two-hybrid system (B2H) comprising a first construct comprising a first promoter P, a first RBS and a reporter gene, the first promoter P allowing a stable basal level of expression of the reporter gene, and a second construct comprising a second promoter P′, a second RBS and a repressor gene, said repressor being capable of targeting the first promoter P to block the transcription of the reporter gene, and the expression of the promoter P′ being controlled the B2H system including FPR and FPL, and the method comprises the selection of the variant encoded by an altered copy of the gene L when the expression of the reporter is decreased, optionally under a predetermined level.

Bacterial two-hybrid (B2H) systems are well known by the person skilled in the art. For instance, examples of B2H are disclosed in WO9825947, McLaughlin et al (2012, Nature, 491, 138-142), Hugh et al (2016, PLOS Pathogen, DOI:10.1371) and Poelwijk et al (2019, Nature Communications, 10, 4213), the disclosure thereof being incorporated herein by reference. In particular, B2H used in the present disclosure can be a B2H system as developed and described by Dove et al (Methods Mol Biol. 2004; 261:231-46) with one of the fusion proteins having transcription activator when its interaction partner is fused to a subunit of the bacterial RNA polymerase.

In a particular aspect, the first partner is a DNA binding domain (DBD) and the second partner is a transcription subunit (TrSu). For instance, the DBD can be cI protein of bacteriophage lambda and may have a sequence of SEQ ID NO: 22 and the transcription activator can be the subunit alpha of the RNA polymerase and may a sequence of SEQ ID NO: 23. Other DBDs and TrSus can be used in order to build two hybrid systems. Theoretically, the great majority of the domain that can bind to DNA could be used as DBD in a B2H set-up. Especially, but not limited to, repressors from different families (such as cI, lad and tetR), zinc-fingers, transcription activator-like effectors (TALE) and dead Cas9 (dCas9). Badran et al (2016, Nature, 533, 58-63) demonstrated the used the DBD from 494 phage cI while Joung et al (2000, PNAS, 97, 7382-7387) demonstrated the use of zinc-finger domains; Yurlova et al the use of lad in a fluorescent two-hybrid assay (2014, Journal of Biomolecule Screening, 19, 516-525); Li, et al the use of TALEs (2012, Scientific Reports, 2, 897) and; Hass & Zappulla the use of dCas9 (DOI: 10.1101/139600). Concerning the use of other Escherichia coli RNA polymerase subunits as TrSus, Dove & Hochschild (1998, Genes & Development, 12, 745-754) and Badran et al (2016, Nature, 533, 58-63) used omega subunit of Escherichia coli RNA polymerase (coded by gene rpoZ). Hennecke et al., (2005, Protein Engineering, Design and Selection, 18, 477-486) also demonstrated the feasibility of a B2H system inspired from toxR that can probe membrane and periplasmic interactions and that employs a domain that encompasses both functions DBD and TrSU without including a bacterial RNA polymerase subunit thus acting as a transcription activator.

In one aspect, the DBD is linked to the target molecule and forms a fusion protein (FPR) while the transcription subunit is linked to the ligand domain encoded by the gene L and its variants and forms a fusion protein (FPL). In an alternative aspect, the transcription subunit is linked to the target molecule and forms a fusion protein (FPR) while the DBD is linked to the ligand domain encoded by the gene L and its variants and forms a fusion protein (FPL).

The DBD and the transcription subunit are selected in order to promote the expression of the reporter gene or the repressor gene when a binding between FPR and FPL occurs, more particularly when a binding of the ligand domain L and the target molecule occurs. The B2H system can be adjusted to be able to select a suitable affinity for the binding of the ligand domain L and the target molecule.

The inventors designed an optimal reporting system for the B2H based on at least three main features that are: a) improved signal-to-noise ratio; b) the good correlation between affinity and the genetic signal generated and; c) the reduction of signal stochasticity. The first is required to reliably distinguish interactions from the basal expression level (or background noise), the second for the trustworthy comparison of affinities and the third to allow the retrieval of reliable information from large scale experiments. This optimized B2H differs from previous known B2H systems by these three properties which are essential for simultaneous large scale analysis of protein-protein interactions.

A first element of this B2H system is the promoter controlling the expression of the reporter gene or the repressor gene. Then, in a more preferred aspect, the reporter gene or the repressor gene of the B2H system is associated with the promoter epB2H (SEQ ID NO: 24) or an derivative thereof as defined below. This particular promoter surprisingly provides an optimal balance between an advantageous strong genetic output, i.e. a stronger reporter signal intensity, and a good correlation between ligand affinity and signal intensity. Furthermore, the designed promoter also invalidates a methylation site that was associated to low frequency expontaneous autoactivation thereby providing more consistent outputs and making it more suitable for molecular evolution applications with large number of cells and for longer selection periods.

In particular, the methylation motif CC(A/T)GG, the methylated nucleotide being in bold, is mutated to invalidate methylation site. In a particular aspect, CCAGG can be substituted by GGCGG. This modification allows more homogeneous transcription among different cells (decreased stochasticity) and a decreased frequency of interaction-independent transcription (undesirable transcription in absence of interaction between fusions).

The promoter comprises a −10 box and a −35 box, the distance between the boxes being between 15 and 19 bases. The sequence between the two boxes has minor effect on promoter activity.

Modifications have been carried out in −10 and −35 boxes for improving recognition by transcription sigma factor, thereby allowing a better signal-to-noise ratio in B2H systems. More particularly, the −10 box has a sequence of GATACT and the −35 box has a sequence of TTGACA.

Finally, the last element of the promoter is the operator, the sequence recognized by the DBD, for instance cI protein. The operator can be selected among OR1, OR2, OR3, OL1, OL2 and OL3 lambda operators. In a particular aspect, the operator is OL2. The centre of the operator is preferably placed 62 bases upstream the transcription start.

Then, the promoter may comprise, from 5′ to 3′, an operator recognized by DBD, an invalidated methylation site, a modified −35 box of sequence of TTGACA, a modified −10 box has a sequence of GATACT. More specifically, the promoter meets one or several of the following features:
centre of the operator is placed about 62 bases upstream the transcription start;
invalidated methylation site has a sequence of GGCGG;
the distance between the −35 and −10 boxes is between 15 and 19 bases; and
the operator is selected among OR1, OR2, OR3, OL1, OL2 and OL3 lambda operators.

In one aspect, the promoter has the following sequence/structure:

Operator-(N)₁₁-GGCGG-N-TTGACA-(N)₁₅₋₁₉-GATACT-(N)₆-
Start,
Invalidated methylation site -35 box -10 box

with N being any base (A, T, C or G).

In a specific aspect, the promoter has an operator selected among OR1, OR2, OR3, OL1, OL2 and OL3 lambda operators operably linked to a sequence

GGCGGCTTGACACTTTATGCTTCCGGCTCGGATACTGTGTGGA (SEQ ID NO 68) or a sequence having at least 80, 85, 90 or 95% of identity with SEQ ID NO 68 and no modification in the region with bold and underlined nucleotides.

In a more specific aspect, the promoter has the following sequence:

(SEQ ID NO 24)
CAACACCGCCAGAGATACATTAGGCACCGGCGGCTTGACACTTTATGCT
TCCGGCTCGGATACTGTGTGGA

or a sequence having at least 80, 85, 90 or 95% of identity with SEQ

ID NO 24 and no modification in the region with bold and underlined nucleotides.

A transcription terminator has been placed upstream the operator element of epB2H promoter in order to avoid that transcription from upstream elements disturbs epB2H regulation. For instance, the terminator last base could be placed between 15 and 53 bases (about 1.5 to 5 DNA helix turns) upstream of the first operator base. More specifically, the terminator last base could be placed 26 bases upstream of the first operator base. The terminator can be selected among small and strong terminators, for instance those disclosed in Chen et al (2013, Nature Methods, 10, 659-666), the disclosure thereof being incorporated herein by reference, in particular the terminators specifically disclosed in Supplementary Tables 2-4 of Chen et al. In a particular aspect, the transcription terminator has the following sequence (SEQ ID NO: 69 CGCAAAAAACCCCGCCCCTGACAGGGCGGGGTTTTTTCGC).

Then, the B2H system of the present invention comprises a promoter as disclosed above and a transcription terminator placed upstream of the first base of the operator.

Preferably, the expression cassette of the reporter gene is on a single and low copy number vector or is integrated into the bacterial genome.

In a more preferred aspect, the expression of the FPR and/or FPL component, optionally the component comprising the DBD, is controlled by the association of a strong promoter and a weak RBS. Accordingly, the sequences of the FPR and/or FPL component, optionally the component comprising the DBD, are operably linked both to a strong promoter and a weak RBS. Interestingly, the inventors show that this association of a strong promoter and a weak RBS decreases the stochastic behaviour, thereby further improving the B2H system. In a particular aspect, the sequences of the FPR and/or FPL component of the B2H system are associated with the weak RBS named RBS7 (SEQ ID NO:20) and the strong promoter pLTetO (SEQ ID NO:21). In a particular aspect, the sequences of the FPR and/or FPL component of the B2H system are operably linked to a combination of the promoter pLTetO with the RB S7 and has the following sequence

(SEQ ID NO 70)
TTGACATCCCTATCAGTGATAGAGATACTGCTAGCACTTAAGTAGACCA
GCTCGCTAGGTCATATA

or a sequence having at least 95% of identity with SEQ ID NO 70 and no modification in the region with bold and underlined nucleotides.

The present invention relates to a bacterial cell comprising the above-mentioned components of the first module and the second module comprising an HR factor as detailed above, and that further comprises the B2H components as detailed herein in any aspect and uses thereof for detecting the interaction between a target molecule and a ligand variant generated from the altered copies of gene L and/or select an altered copies of gene L for its interacting abilities. The present invention also relates to a bacterial cell comprising the above-mentioned components of the third module, especially with its improved and optimal aspects.

In one aspect, the present invention relates to a method for screening a ligand molecule capable of binding a target molecule from variants encoded by altered copies of a gene L, comprising any aspects of the steps described for the module 1 and steps described for module 2 wherein the module 2 comprises an HR factor, wherein the provided bacterial cell further comprises a B2H system comprising:
a promoter (P), a sequence defining a ribosome binding site (RBS) and a reporter gene, the P sequence being operably linked to the RBS sequence and the reporter gene,
a fusion protein (FPR) comprising the target molecule and a DNA binding domain (DBD), said DBD being capable of binding to a site located at proximity of the promoter P so as to promote the expression of the reporter gene when the target molecule is bound to a variant encoded by an altered copy of the gene L, and
a fusion protein (FPL) comprising a variant encoded by an altered copy of the gene L and transcription subunits (TrSu) capable of recruiting an RNA polymerase, and.

the method comprises the selection of the variant encoded by an altered copy of the gene L when the reporter is expressed, optionally at least at a predetermined level.

Preferably, the B2H comprises a strong promoter and a weak RBS operably linked to the FPR and/or FPL component, preferably FPR. In a particular aspect, the sequences of the FPR and/or FPL component, preferably FPR, of the B2H system are associated with the weak RBS named RBS7 (SEQ ID NO:20) and the strong promoter pLTetO (SEQ ID NO:21). In a particular aspect, the sequences of the FPR and/or FPL component of the B2H system are operably linked to a combination of the promoter pLTetO with the RB S7 and has the following sequence:

(SEQ ID NO 70)
TTGACATCCCTATCAGTGATAGAGATACTGCTAGCACTTAAGTAGACCA
GCTCGCTAGGTCATATA

or a sequence having at least 95% of identity with SEQ ID NO 70 and no modification in the region with bold and underlined nucleotides.

Preferably, the promoter P is the promoter epB2H (SEQ ID NO: 24) or a derivative thereof as detailed above. Accordingly, the promoter P has the following structure:

Operator-(N)₁₁-GGCGG-N-TTGACA-(N)₁₅₋₁₉-GATACT-(N)₆-
Start,

with operator being the sequence recognized by DBD, Start being the nucleotide where the transcription starts, and N being any base (A, T, C or G).

In a preferred aspect, a transcription terminator is placed upstream the operator, preferably of a transcription terminator having a sequence as shown in SEQ ID NO: 69.

In a more specific aspect, the DBD is a cI protein and the promoter P has an operator selected among OR1, OR2, OR3, OL1, OL2 and OL3 lambda operators operably linked to a sequence

GGCGGCTTGACACTTTATGCTTCCGGCTCGGATACTGTGTGGA (SEQ ID NO: 68) or a sequence having at least 80, 85, 90 or 95% of identity with SEQ ID NO: 68 and no modification in the region with bold and underlined nucleotides.

In an even more specific aspect, the DBD is a cI protein and the promoter P has the following sequence:

(SEQ ID NO: 24)
CAACACCGCCAGAGATACATTAGGCACCGGCGGCTTGACACTTTATGCTTC
CGGCTCGGATACTGTGTGGA

or a sequence having at least 80, 85, 90 or 95% of identity with SEQ ID NO: 24 and no modification in the region with bold and underlined nucleotides.

In an alternative aspect, the present invention relates to a method for screening a ligand molecule capable of binding a target molecule from variants encoded by altered copies of a gene L, comprising any aspects of the steps described for the module 1 and steps described for module 2 wherein the module 2 comprises an HR factor, wherein the provided bacterial cell further comprises a B2H system comprising:
a promoter (P), a sequence defining a ribosome binding site (RBS) and a reporter gene, the P sequence being operably linked to the RBS sequence and the reporter gene,
a fusion protein (FPR) comprising the target molecule and transcription subunits (TrSu) capable of recruiting an RNA polymerase, and
a fusion protein (FPL) comprising a variant encoded by an altered copy of the gene L and a DNA binding domain (DBD), said DBD being capable of binding to a site located at proximity of the promoter P so as to promote the expression of the reporter gene when the target molecule is bound to a variant encoded by an altered copy of the gene L, and

the method comprises the selection of the variant encoded by an altered copy of the gene L when the reporter is expressed, optionally at least at a predetermined level.

Alternatively, when the method is for screening a ligand molecule that loses the binding capacity to a target molecule from variants encoded by altered copies of a gene L, the method comprises the selection of the variant encoded by an altered copy of the gene L when the reporter is decreased, optionally under a predetermined level.

Preferably, the B2H comprises a strong promoter and a weak RBS operably linked to the FPR and/or FPL component, preferably FPL. In a particular aspect, the sequences of the FPR and/or FPL component, preferably FPL, of the B2H system are associated with the weak RBS named RBS7 (SEQ ID NO: 20) and the strong promoter pLTetO (SEQ ID NO: 21). In an alternative aspect, the sequences of the FPR and/or FPL component of the B2H system are operably linked to a combination of the promoter pLTetO with the RB S7 and has the following sequence

(SEQ ID NO 70)
TTGACATCCCTATCAGTGATAGAGATACTGCTAGCACTTAAGTAGACCA
GCTCGCTAGGTCATATA

or a sequence having at least 95% of identity with SEQ ID NO 70 and no modification in the region with bold and underlined nucleotides.

Preferably, the promoter P is the promoter epB2H (SEQ ID NO: 24) or an alternative thereof.

Accordingly, the promoter P has the following structure:

Operator - (N)₁₁-GGCGG-N-TTGACA-(N)₁₅₋₁₉-GATACT-
(N)₆-Start,

with operator being the sequence recognized by DBD, Start being the nucleotide where the transcription starts, and N being any base (A, T, C or G).

In a preferred aspect, a transcription terminator is placed upstream the operator, preferably of a transcription terminator having a sequence as shown in SEQ ID NO: 69.

In a more specific aspect, the DBD is a cI protein and the promoter P has an operator selected among OR1, OR2, OR3, OL1, OL2 and OL3 lambda operators operably linked to a sequence GGCGGCTTGACACTTTATGCTTCCGGCTCGGATACTGTGTGGA (SEQ ID NO: 68) or a sequence having at least 80, 85, 90 or 95% of identity with SEQ ID NO: 68 and no modification in the region with bold and underlined nucleotides.

In an even more specific aspect, the DBD is a cI protein and the promoter P has the following sequence

(SEQ ID NO: 24)
CAACACCGCCAGAGATACATTAGGCACCGGCGGCTTGACACTTTATGCTT
CCGGCTCGGATACTGTGTGGA

or a sequence having at least 80, 85, 90 or 95% of identity with SEQ ID NO: 24 and no modification in the region with bold and underlined nucleotides.

The present invention also relates to a method for screening a ligand molecule that loses the capacity of binding a target molecule from variants encoded by altered copies of a gene L, comprising any aspects of the steps described for the module 1 and steps described for module 2 wherein the module 2 comprises an HR factor as detailed above, wherein the provided bacterial cell further comprises a B2H system comprising:
a first promoter P, a sequence defining a first ribosome binding site (RBS) and a reporter gene, the first promoter P being operably linked to the first RBS sequence and the reporter gene and allowing a stable basal level of expression of the reporter gene, and
a second promoter P′, a sequence defining a second RBS and a repressor gene, the second promoter P′ being operably linked to the second RBS sequence and the repressor gene, said repressor being capable of targeting the first promoter P to block the transcription of the reporter gene,
a fusion protein (FPR) comprising the target molecule and a DNA binding domain (DBD), said DBD being capable of binding to a site located at proximity of the promoter P′ so as to promote the expression of the repressor gene when the target molecule is bound to a variant encoded by an altered copy of the gene L, and
a fusion protein (FPL) comprising a variant encoded by an altered copy of the gene L and transcription subunits (TrSu) capable of recruiting an RNA polymerase; and the method comprises the selection of the variant encoded by an altered copy of the gene L when the expression of the reporter is increased, optionally at least to a predetermined level.

Preferably, the B2H comprises a strong promoter and a weak RBS operably linked to the FPR and/or FPL component, preferably FPR. In a particular aspect, the sequences of the FPR and/or

FPL component, preferably FPR, of the B2H system are associated with the weak RBS named RBS7 (SEQ ID NO: 20) and the strong promoter pLTetO (SEQ ID NO: 21). In an alternative aspect, the sequences of the FPR and/or FPL component of the B2H system are operably linked to a combination of the promoter pLTetO with the RB S7 and has the following sequence

(SEQ ID NO 70)
TTGACATCCCTATCAGTGATAGAGATACTGCTAGCACTTAAGTAGACCAG
CTCGCTAGGTCATATA

or a sequence having at least 95% of identity with SEQ ID NO 70 and no modification in the region with bold and underlined nucleotides.

Preferably, the promoter P′ is the promoter epB2H (SEQ ID NO: 24) or a derivative thereof as defined above. For instance, the promoter P′ has the following sequence:

(SEQ ID NO: 24)
CAACACCGCCAGAGATACATTAGGCACCGGCGGCTTGACACTTTATGCTT
CCGGCTCGGATACTGTGTGGA

or a sequence having at least 80, 85, 90 or 95% of identity with SEQ ID NO 24 and no modification in the region with bold and underlined nucleotides.

Optionally, the repressor could be SrpR and the promoter P could be T7-SprOx2.

Alternatively, the present invention also relates to a method for screening a ligand molecule that loses capable the capacity of binding a target molecule from variants encoded by altered copies of a gene L, comprising any aspects of the steps described for the module 1 and steps described for module 2 wherein the module 2 comprises an HR factor as detailed above, wherein the provided bacterial cell further comprises a B2H system comprising:
a first promoter P, a sequence defining a first ribosome binding site (RBS) and a reporter gene, the first promoter P being operably linked to the first RBS sequence and the reporter gene and allowing a stable basal level of expression of the reporter gene, and
a second promoter P′, a sequence defining a second RBS and a repressor gene, the second promoter P′ being operably linked to the second RBS sequence and the repressor gene, said repressor being capable of targeting the first promoter P to block the transcription of the reporter gene,
a fusion protein (FPR) comprising the target molecule and transcription subunits (TrSu) capable of recruiting an RNA polymerase, and
a fusion protein (FPL) comprising a variant encoded by an altered copy of the gene L and a DNA binding domain (DBD), said DBD being capable of binding to a site located at proximity of the promoter P′ so as to promote the expression of the repressor gene when the target molecule is bound to a variant encoded by an altered copy of the gene L; and

the method comprises the selection of the variant encoded by an altered copy of the gene L when the expression of the reporter is increased, optionally at least at a predetermined level.

Preferably, the B2H comprises a strong promoter and a weak RBS operably linked to the FPR and/or FPL component, preferably FPL. In a particular aspect, the sequences of the FPR and/or FPL component, preferably FPL, of the B2H system are associated with the weak RBS named RBS7 (SEQ ID NO: 20) and the strong promoter pLTetO (SEQ ID NO: 21). In an alternative aspect, the sequences of the FPR and/or FPL component of the B2H system are operably linked to a combination of the promoter pLTetO with the RB S7 and has the following sequence

(SEQ ID NO 70)
TTGACATCCCTATCAGTGATAGAGATACTGCTAGCACTTAAGTAGACCAG
CTCGCTAGGTCATATA

or a sequence having at least 95% of identity with SEQ ID NO 70 and no modification in the region with bold and underlined nucleotides.

Preferably, the promoter P′ is the promoter epB2H (SEQ ID NO: 24) or a derivative thereof as disclosed above. For instance, the promoter P′ has the following sequence:

(SEQ ID NO: 24)
CAACACCGCCAGAGATACATTAGGCACCGGCGGCTTGACACTTTATGCTT
CCGGCTCGGATACTGTGTGGA

or a sequence having at least 80, 85, 90 or 95% of identity with SEQ ID NO 24 and no modification in the region with bold and underlined nucleotides.

The present invention further relates to a vector or set of vectors as described above for module 1 and module 2 including HR, to a bacterial comprising said vector or set of vectors, and to the use of said vector or set of vectors or said bacterial cell, said vector or set of vectors further comprising:
an expression cassette (eC4) comprising a sequence encoding a reporter gene operably linked to a promoter (P6),
an expression cassette (eC5) comprising a sequence encoding an FPR protein operably linked to a promoter (P7), said FPR comprising a target domain and a DBD sequence, said DBD being capable of binding to a site located at proximity of the promoter P6 so as to promote the expression of the reporter gene when the target molecule is bound to a variant encoded by an altered copy of the gene L, wherein said eC5 is suitable for allowing, in the bacterial cell, the expression of an FPR protein, and
an expression cassette (eC6) comprising a sequence encoding an FPL protein operably linked to a promoter (P8), said FPL comprising an insertion site suitable for the insertion of the gene L and transcription subunits (TrSu) capable of recruiting an RNA polymerase, wherein said eC6 is suitable for allowing, in the bacterial cell, the expression of a FPL protein comprising either a ligand encoded by the gene L or a variant thereof encoded by an HR-integrated altered copy of gene L;

or
an expression cassette (eC4) comprising a sequence encoding a reporter gene operably linked to a promoter (P6),
an expression cassette (eC5) comprising a sequence encoding an FPR protein operably linked to a promoter (P7), said FPR comprising a target domain and transcription subunits (TrSu) capable of recruiting an RNA polymerase, wherein said eC5 is suitable for allowing, in the bacterial cell, the expression of an FPR protein, and
an expression cassette (eC6) comprising a sequence encoding an FPL protein operably linked to a promoter (P8), said FPL comprising an insertion site suitable for the insertion of the gene L and a DBD sequence, said DBD being capable of binding to a site located at proximity of the promoter P6 so as to promote the expression of the reporter gene when the target molecule is bound to a variant encoded by an altered copy of the gene L, wherein said eC6 is suitable for allowing, in the bacterial cell, the expression of an FPL protein comprising either a ligand encoded by the gene L or a variant thereof encoded by an HR-integrated altered copy of gene L;

or
an expression cassette (eC4) comprising a sequence encoding a reporter gene operably linked to a promoter (P6),
an expression cassette (eC5) comprising a sequence encoding an FPR protein operably linked to a promoter (P7), said FPR comprising a target domain and a DBD sequence, said DBD being capable of binding to a site located at proximity of the promoter P6 so as to promote the expression of the reporter gene when the target molecule is bound to a variant encoded by an altered copy of the gene L, wherein said eC5 is suitable for allowing, in the bacterial cell, the expression of an FPR protein, and
an expression cassette (eC6) comprising a sequence encoding an FPL protein operably linked to a promoter (P8), said FPL comprising the gene L and transcription subunits (TrSu) capable of recruiting an RNA polymerase, wherein said eC6 is suitable for allowing, in the bacterial cell, the expression of a FPL protein comprising either a ligand encoded by the gene L or a variant thereof encoded by an HR-integrated altered copy of gene L;

or
an expression cassette (eC4) comprising a sequence encoding a reporter gene operably linked to a promoter (P6),
an expression cassette (eC5) comprising a sequence encoding an FPR protein operably linked to a promoter (P7), said FPR comprising a target domain and transcription subunits (TrSu) capable of recruiting an RNA polymerase, wherein said eC5 is suitable for allowing, in the bacterial cell, the expression of an FPR protein, and
an expression cassette (eC6) comprising a sequence encoding an FPL protein operably linked to a promoter (P8), said FPL comprising the gene L and a DBD sequence, said DBD being capable of binding to a site located at proximity of the promoter P6 so as to promote the expression of the reporter gene when the target molecule is bound to a variant encoded by an altered copy of the gene L, wherein said eC6 is suitable for allowing, in the bacterial cell, the expression of an FPL protein comprising either a ligand encoded by the gene L or a variant thereof encoded by an HR-integrated altered copy of gene L.

The present invention also relates to a vector or set of vectors as described above, that further comprises:
an expression cassette (eC4) comprising a sequence encoding a repressor gene operably linked to a promoter (P6),
an expression cassette (eC4′) comprising a sequence encoding a reporter gene operably linked to a promoter (P6′), the expression of the reporter gene being negatively controlled by the repressor encoded by (eC4),
an expression cassette (eC5) comprising a sequence encoding an FPR protein operably linked to a promoter (P7), said FPR comprising a target domain and a DBD sequence, said DBD being capable of binding to a site located at proximity of the promoter P6 so as to promote the expression of the repressor gene when the target molecule is bound to a variant encoded by an altered copy of the gene L, wherein said eC5 is suitable for allowing, in the bacterial cell, the expression of an FPR protein, and
an expression cassette (eC6) comprising a sequence encoding an FPL protein operably linked to a promoter (P8), said FPL comprising an insertion site suitable for the insertion of the gene L and transcription subunits (TrSu) capable of recruiting an RNA polymerase, wherein said eC6 is suitable for allowing, in the bacterial cell, the expression of a FPL protein comprising either a ligand encoded by the gene L or a variant thereof encoded by an HR-integrated altered copy of gene L;

or
an expression cassette (eC4) comprising a sequence encoding a repressor gene operably linked to a promoter (P6),
an expression cassette (eC4′) comprising a sequence encoding a reporter gene operably linked to a promoter (P6′), the expression of the reporter gene being negatively controlled by the repressor encoded by (eC4),
an expression cassette (eC5) comprising a sequence encoding an FPR protein operably linked to a promoter (P7), said FPR comprising a target domain and transcription subunits (TrSu) capable of recruiting an RNA polymerase, wherein said eC5 is suitable for allowing, in the bacterial cell, the expression of an FPR protein, and
an expression cassette (eC6) comprising a sequence encoding an FPL protein operably linked to a promoter (P8), said FPL comprising an insertion site suitable for the insertion of the gene L and a DBD sequence, said DBD being capable of binding to a site located at proximity of the promoter P6 so as to promote the expression of the repressor gene when the target molecule is bound to a variant encoded by an altered copy of the gene L, wherein said eC6 is suitable for allowing, in the bacterial cell, the expression of an FPL protein comprising either a ligand encoded by the gene L or a variant thereof encoded by an HR-integrated altered copy of gene L;

or
an expression cassette (eC4) comprising a sequence encoding a repressor gene operably linked to a promoter (P6),
an expression cassette (eC4′) comprising a sequence encoding a reporter gene operably linked to a promoter (P6′), the expression of the reporter gene being negatively controlled by the repressor encoded by (eC4),
an expression cassette (eC5) comprising a sequence encoding an FPR protein operably linked to a promoter (P7), said FPR comprising a target domain and a DBD sequence, said DBD being capable of binding to a site located at proximity of the promoter P6 so as to promote the expression of the repressor gene when the target molecule is bound to a variant encoded by an altered copy of the gene L, wherein said eC5 is suitable for allowing, in the bacterial cell, the expression of an FPR protein, and
an expression cassette (eC6) comprising a sequence encoding an FPL protein operably linked to a promoter (P8), said FPL comprising the gene L and transcription subunits (TrSu) capable of recruiting an RNA polymerase, wherein said eC6 is suitable for allowing, in the bacterial cell, the expression of a FPL protein comprising either a ligand encoded by the gene L or a variant thereof encoded by an HR-integrated altered copy of gene L;

or
an expression cassette (eC4) comprising a sequence encoding a repressor gene operably linked to a promoter (P6),
an expression cassette (eC4′) comprising a sequence encoding a reporter gene operably linked to a promoter (P6′), the expression of the reporter gene being negatively controlled by the repressor encoded by (eC4),
an expression cassette (eC5) comprising a sequence encoding an FPR protein operably linked to a promoter (P7), said FPR comprising a target domain and transcription subunits (TrSu) capable of recruiting an RNA polymerase, wherein said eC5 is suitable for allowing, in the bacterial cell, the expression of an FPR protein, and
an expression cassette (eC6) comprising a sequence encoding an FPL protein operably linked to a promoter (P8), said FPL comprising the gene L and a DBD sequence, said DBD being capable of binding to a site located at proximity of the promoter P6 so as to promote the expression of the repressor gene when the target molecule is bound to a variant encoded by an altered copy of the gene L, wherein said eC6 is suitable for allowing, in the bacterial cell, the expression of an FPL protein comprising either a ligand encoded by the gene L or a variant thereof encoded by an HR-integrated altered copy of gene L.

Optionally, the promoter P7 and/or P8 comprises a strong promoter and a weak RBS, in particular a weak RBS named RBS7 (SEQ ID NO: 20) and a strong promoter such as pLTetO (SEQ ID NO: 21). In a particular aspect, the sequences of the FPR and/or FPL component of the B2H system are operably linked to a combination of the promoter pLTetO with the RBS7 and has the following sequence

(SEQ ID NO 70)
TTGACATCCCTATCAGTGATAGAGATACTGCTAGCACTTAAGTAGACCAG
CTCGCTAGGTCATATA

or a sequence having at least 95% of identity with SEQ ID NO 70 and no modification in the region with bold and underlined nucleotides.

Optionally, the promoter P6 or P6′ is the promoter epB2H (SEQ ID NO: 24) or an alternative thereof.

Accordingly, the promoter P6 or P6′ has the following structure:

Operator - (N)₁₁-GGCGG-N-TTGACA-(N)₁₅₋₁₉-GATACT-
(N)₆-Start,

with operator being the sequence recognized by DBD, Start being the nucleotide where the transcription starts, and N being any base (A, T, C or G).

In a preferred aspect, a transcription terminator is placed upstream the operator, preferably of a transcription terminator having a sequence as shown in SEQ ID NO: 69.

In a more specific aspect, the DBD is a cI protein and the promoter P6 or P6′ has an operator selected among OR1, OR2, OR3, OL1, OL2 and OL3 lambda operators operably linked to a sequence

GGCGGCTTGACACTTTATGCTTCCGGCTCGGATACTGTGTGGA (SEQ ID NO: 68) or a sequence having at least 80, 85, 90 or 95% of identity with SEQ ID NO: 68 and no modification in the region with bold and underlined nucleotides.

In an even more specific aspect, the DBD is a cI protein and the promoter P6 or P6′ has the following sequence

(SEQ ID NO: 24)
CAACACCGCCAGAGATACATTAGGCACCGGCGGCTTGACACTTTATGCTT
CCGGCTCGGATACTGTGTGGA

or a sequence having at least 80, 85, 90 or 95% of identity with SEQ ID NO: 24 and no modification in the region with bold and underlined nucleotides.

Preferably the vector or the set of vectors is low copy vector.

The present invention also relates to the B2H system with the improvements and its uses, independently of the modules 1 and 2.

Accordingly, the present invention relates to a method for determining a capacity of a ligand molecule and variants of the ligand molecule of binding a target molecule in a bacterial cell, wherein the bacterial cell comprises a two-hybrid system (B2H) comprising:
a promoter (P), a sequence defining a ribosome binding site (RBS) and a reporter gene, the P sequence being operably linked to the RBS sequence and the reporter gene,

and
a fusion protein (FPR) comprising the target molecule and a DNA binding domain (DBD), said DBD being capable of binding to a site located at proximity of the promoter P so as to promote the expression of the reporter gene when the target molecule is bound to the ligand molecule or a variant thereof, and
a fusion protein (FPL) comprising the ligand molecule or a variant thereof and transcription subunits (TrSu) capable of recruiting an RNA polymerase,

or
a fusion protein (FPL) comprising the ligand molecule or a variant thereof and a DNA binding domain (DBD), said DBD being capable of binding to a site located at proximity of the promoter P so as to promote the expression of the reporter gene when the target molecule is bound to the ligand molecule or a variant thereof, and
a fusion protein (FPR) comprising the target molecule and transcription subunits (TrSu) capable of recruiting an RNA polymerase,

and the method comprises the measure of the level of expression of the reporter gene, thereby determining the capacity of a ligand molecule and a variant thereof of binding a target molecule;

wherein the promoter (P) has the following structure:

Operator - (N)₁₁-GGCGG-N-TTGACA-(N)₁₅₋₁₉-GATACT-
(N)₆-Start,

with operator being the sequence recognized by DBD, Start being the nucleotide where the transcription starts, and N being any base (A, T, C or G); and

wherein the fusion protein comprising DBD is operably linked to a strong promoter and a weak RBS.

Preferably, a transcription terminator is placed upstream the operator, preferably of a transcription terminator having a sequence as shown in SEQ ID NO: 69.

In a particular aspect, the DBD is a cI protein and the promoter (P) has an operator selected among OR1, OR2, OR3, OL1, OL2 and OL3 lambda operators operably linked to a sequence

GGCGGCTTGACACTTTATGCTTCCGGCTCGGATACTGTGTGGA (SEQ ID NO: 68) or a sequence having at least 80, 85, 90 or 95% of identity with SEQ ID NO: 68 and no modification in the region with bold and underlined nucleotides.

In a more particular aspect, wherein the DBD is a cI protein and the promoter (P) has the following sequence

(SEQ ID NO: 24)
CAACACCGCCAGAGATACATTAGGCACCGGCGGCTTGACACTTTATGCTT
CCGGCTCGGATACTGTGTGGA

or a sequence having at least 80, 85, 90 or 95% of identity with SEQ ID NO: 24 and no modification in the region with bold and underlined nucleotides.

Optionally, the strong promoter with the weak RBS has the following sequence

(SEQ ID NO 70)
TTGACATCCCTATCAGTGATAGAGATACTGCTAGCACTTAAGTAGACCAG
CTCGCTAGGTCATATA

or a sequence having at least 95% of identity with SEQ ID NO 70 and no modification in the region with bold and underlined nucleotides.

Optionally, the weak RBS has the sequence as shown in SEQ ID NO: 20 and the strong promoter has a sequence as shown in SEQ ID NO: 21.

Optionally, the method comprises the comparison of the level of expression of the reporter gene of the ligand molecule to the level of expression of the reporter gene of the variant, thereby determining the effect of the modification in the variant on the binding to the target molecule.

The present invention relates to any use of the method in any kind of applications. For instance, this B2H system is well-adapted interface mapping of interacting proteins. This system is well adapted to the Deep mutational scanning. Then, in a particular aspect, the present invention relates to a method for mapping amino acids in two interacting molecules (ligand and target), wherein variants of the ligand are prepared and the effect of the amino acid substitution(s) on their interaction with the target protein is determined by the method as detailed above. The variants of the ligand can be generated by Deep mutational scanning, in which selected amino acid positions are substituted by one or several amino acids, preferably by all amino acids.

The present invention also relates to a B2H system for determining a capacity of a ligand molecule and variants of the ligand molecule of binding a target molecule comprising a bacterial cell comprising following expression cassettes
an expression cassette (eC4) comprising a sequence encoding a reporter gene operably linked to a promoter (P6),
an expression cassette (eC5) comprising a sequence encoding an FPR protein operably linked to a promoter (P7), said FPR comprising a target domain and a DBD sequence, said DBD being capable of binding to a site located at proximity of the promoter P6 so as to promote the expression of the reporter gene when the target molecule is bound to a ligand molecule or a variant thereof, wherein said eC5 is suitable for allowing, in the bacterial cell, the expression of an FPR protein, and
an expression cassette (eC6) comprising a sequence encoding an FPL protein operably linked to a promoter (P8), said FPL comprising the ligand molecule or a variant thereof and transcription subunits (TrSu) capable of recruiting an RNA polymerase, wherein said eC6 is suitable for allowing, in the bacterial cell, the expression of an FPL protein;

or
an expression cassette (eC4) comprising a sequence encoding a reporter gene operably linked to a promoter (P6),
an expression cassette (eC5) comprising a sequence encoding an FPR protein operably linked to a promoter (P7), said FPR comprising the ligand molecule or a variant thereof and a DBD sequence, said DBD being capable of binding to a site located at proximity of the promoter P6 so as to promote the expression of the reporter gene when the target molecule is bound to a ligand molecule or a variant thereof, wherein said eC5 is suitable for allowing, in the bacterial cell, the expression of an FPR protein, and
an expression cassette (eC6) comprising a sequence encoding an FPL protein operably linked to a promoter (P8), said FPL comprising the target molecule and transcription subunits (TrSu) capable of recruiting an RNA polymerase, wherein said eC6 is suitable for allowing, in the bacterial cell, the expression of an FPL protein;

wherein the promoter (P6) has the following structure:

Operator - (N)₁₁-GGCGG-N-TTGACA-(N)₁₅₋₁₉-GATACT-
(N)₆-Start,

with operator being the sequence recognized by DBD, Start being the nucleotide where the transcription starts, and N being any base (A, T, C or G); and

wherein the promoter (P7) and/or the promoter (P8) is/are a strong promoter operably linked to a weak RBS.

Preferably, a transcription terminator is placed upstream the operator, preferably of a transcription terminator having a sequence as shown in SEQ ID NO: 69.

In a more specific aspect, the DBD is a cI protein and the promoter P6 has an operator selected among OR1, OR2, OR3, OL1, OL2 and OL3 lambda operators operably linked to a sequence

GGCGGCTTGACACTTTATGCTTCCGGCTCGGATACTGTGTGGA (SEQ ID NO: 68) or a sequence having at least 80, 85, 90 or 95% of identity with SEQ ID NO: 68 and no modification in the region with bold and underlined nucleotides.

In an even more specific aspect, the DBD is a cI protein and the promoter P6 has the following sequence:

(SEQ ID NO: 24)
CAACACCGCCAGAGATACATTAGGCACCGGCGGCTTGACACTTTATGCTT
CCGGCTCGGATACTGTGTGGA

or a sequence having at least 80, 85, 90 or 95% of identity with SEQ ID NO: 24 and no modification in the region with bold and underlined nucleotides.

In a particular aspect, the sequences of the FPR and/or FPL component, preferably FPL, of the B2H system are associated with the weak RBS named RBS7 (SEQ ID NO: 20) and the strong promoter pLTetO (SEQ ID NO: 21). In a particular aspect, the sequences of the FPR and/or FPL component of the B2H system are operably linked to a combination of the promoter pLTetO with the RBS7 and has the following sequence

(SEQ ID NO 70)
TTGACATCCCTATCAGTGATAGAGATACTGCTAGCACTTAAGTAGACCAG
CTCGCTAGGTCATATA

or a sequence having at least 95% of identity with SEQ ID NO 70 and no modification in the region with bold and underlined nucleotides.

### Module 4: Arrest of the Evolution

The fourth module comprises means for allowing to stop the generation of diversity carried out by the first and second modules of the disclosure.

The fourth module is an optional module that can be added to the combination of the three other modules in order to stop the evolution process, in particular when a ligand of interest has been generated in a bacterial cell. The advantage of stopping the generation of diversity by using the fourth module is the possibility to preserve the altered copy of the gene L that is expressed by the B2H system, i.e. by avoiding its replacement by another variant of the gene L. In addition, although generation of diversity is stopped by the fourth module, the expression of the selected variant and its detection by the third module continue, thus allowing the isolation of the corresponding cells, and the identification and characterization of the variant by suitable techniques known by the person skilled in the art.

In particular, the fourth module is functionally coupled to the B2H system of the third module. This functional coupling results from the fact that the sequence coding for the arrest factor of the fourth module is operationally linked to a promoter controlled by the B2H, especially the reporter gene and to its promoter or the repressor gene and its promoter. In other words, the expression of this arrest factor depends on the binding or non-binding between FPL and FPR. By “arrest factor” of the fourth module is intended to refer to proteins such as enzyme that actively triggers the arrest of the generation of diversity. In addition, other elements can cooperate with the arrest factor in order to allow the arrest of the generation of diversity.

The arrest factor of the fourth module impairs the HR function and/or the RT function. In a more preferred aspect, the arrest factor of the fourth module impairs both the HR function and the RT function. Impairment of the RT function allows to abolish the generation of altered copies of the gene L while the impairment of the HR function allows to prevent these altered copies from being integrated in an expression cassette of the FPL or FPR of the B2H system.

In a preferred aspect, the arrest factor of the fourth module is expressed by the B2H system of the third module when the latter detects a binding between the FPL and the FPR. According to this aspect, an effective ligand variant is generated from an original gene L that codes for an ineffective ligand. The arrest of the generation of diversity then favours the identification of this effective ligand variant. In this aspect, the expression of the arrest factor is controlled by the promoter of the reporter gene or the repressor gene.

The sequence encoding the arrest factor can then be expressed by a polycistronic construct allowing the expression of the reporter gene and the arrest factor or the expression of the repressor gene and the arrest factor. Alternatively, the expression of the reporter or repressor gene and of the arrest factor can be controlled by similar but distinct promoters, all controlled by the B2H system.

Optionally, the arrest factor is an invertase. In a particular aspect, the fourth module comprises a DNA invertase that recognizes DNA sequences that are flanked by a pair of DNA invertase sites.

According to this aspect, the expression of the DNA invertase is controlled by the B2H system and the DNA invertases sites flank DNA sequence coding the RT and/or DNA sequence coding the HR, thereby allowing their targeting by the DNA invertase. Optionally, the DNA invertase can be the BxB1 DNA invertase (e.g., SEQ ID NO: 25) and the DNA invertase sites correspond to Bxb1 attB (e.g., SEQ ID NO: 26) and Bxb1 attP (e.g., SEQ ID NO: 27). More particularly, attP is located in the reverse/complementary strain of the attB sequence. Other invertases and DNA invertase sites are known by the person skilled in the art and can be used in the fourth module.

Alternatively, the arrest factor can be a highly specific restriction enzyme. By highly specific, it refers to restriction enzymes having a long recognition site, preferable at least 11, 12, 13, 14, 15, 16, 17, 18, 19 or 20 bp. In a particular aspect, the fourth module comprises a highly specific restriction enzyme that recognizes DNA sequences that are flanked by a pair of restriction enzyme sites. According to this aspect, the expression of the restriction enzyme is controlled by the B2H system and the restriction enzyme sites flank DNA sequence coding the RT and/or DNA sequence coding the HR factor, thereby allowing their targeting by the restriction enzyme. Once the binding between the target molecule and the ligand molecule occurs, restriction enzyme introduces double-stranded break at restriction sites that flank DNA sequences encoding the RT and/or the HR factor and thereby remove the DNA sequences encoding the RT and/or the HR factor. The restriction enzyme can be wildtype such as I-SceI, I-CreI and the like or artificial such as Zinc finger nucleases or meganucleases, especially of the LAGLIDADG family.

In another alternative, the method for generating diversity in the gene L can be stopped by using a transcription repressor. In this aspect, the B2H further comprises a gene encoding a transcription repressor to the promoter P or P′, and this transcription repressor is capable of stopping or repressing the expression of the DNA sequences encoding the RT and/or the HR factor, thereby stopping the method for generating diversity in a gene L once the binding between the target molecule and the ligand molecule occurs. Optionally, the repressor under the control of the second promoter P′ could be capable of stopping the expression of the DNA sequences encoding the RT and/or the HR factor. In other words, the expression of the DNA sequences encoding the RT and/or the HR factor can be controlled by the repressor under the control of the second promoter P′.

The present invention relates to a bacterial cell comprising the above-mentioned components of the first module, the second module including HR, the components of the third module, that further comprises at least one arrest factor of the fourth module in any aspect and uses thereof for leading to the arrest of the generation of diversity in a gene L.

The present invention relates to a method for screening a ligand molecule capable of binding a target molecule from variants encoded by altered copies of a gene L, comprising any aspect of the previously described steps of the methods implementing Module 3, wherein the B2H system further comprises at least one arrest factor according to the fourth module, preferably a DNA invertase such as the Bxb1 DNA invertase capable of targeting DNA invertase sites that flank DNA sequences encoding the RT and/or the HR; or a restriction enzyme such as I-SceI capable of introduces double-stranded breaks at restriction sites that flank DNA sequences encoding the RT and/or the HR factor and thereby of removing the DNA sequences encoding the RT and/or the HR factor; or a transcription repressor capable of stopping or repressing the expression of the DNA sequences encoding the RT and/or the HR factor.

In a first aspect, the present invention further relates to a vector or set of vectors as described for modules 1, 2 and 3 and said vector or set of vectors have the following features:
a sequence encoding a DNA invertase gene operably linked to P6 in the eC4 expression cassette; and,
DNA invertase sites flanking the sequence encoding the RT and/or the HR, respectively in the eC1 and eC3 expression cassettes.

In a second aspect, the present invention further relates to a vector or set of vectors as described for modules 1, 2 and 3 and said vector or set of vectors have the following features:
a sequence encoding a restriction enzyme gene operably linked to P6 in the eC4 expression cassette; and,
restriction enzyme sites flanking the sequence encoding the RT and/or the HR, respectively in the eC1 and eC3 expression cassettes.

In a third aspect, the present invention further relates to a vector or set of vectors as described for modules 1, 2 and 3 and said vector or set of vectors have the following features:
a sequence encoding a transcription repressor gene operably linked to P6 in the eC4 expression cassette; and,
the sequence encoding the RT and/or the HR, respectively in the eC1 and eC3 expression cassettes can be negatively controlled by the transcription repressor.

The present invention also relates to a bacterial cell comprising the vector or set of vectors as described above with:
a sequence encoding a DNA invertase gene operably linked to P6 in the eC4 expression cassette; and DNA invertase sites flanking the sequence encoding the RT and/or the HR, respectively in the eC1 and eC3 expression cassettes; or.
a gene encoding a highly specific restriction enzyme (such as SceI) linked to the promoter P6 in the eC4 expression cassette, and the eC1 further comprises restriction sites flanking the sequence encoding RBD-RT and/or the eC3 further comprises restriction sites flanking the sequence encoding HR factor gene; or
the eC4 further comprises a sequence encoding a transcription repressor gene operably linked to P6, and the expression of the sequence encoding RBD-RT of the eC1 and/or the sequence encoding HR factor gene of the eC3 can be stopped or negatively controlled by said transcription repressor gene.

Preferably the vector or the set of vectors is low copy vector.

### Bacterial Cells

The present invention relates to a recombinant bacterial cell comprising elements of modules 1, 2, 3 and 4, of modules 1, 2 and 3 or of modules 1 and 2, in particular the vector or set of vectors as defined in any of the modules 1, 2, 3 and 4.

The bacterial cell can be any prokaryotic cell suitable for having functional modules 1, 2, 3 or 4. For instance, bacterial cells could belong to Escherichia coli, Vibrio natriegens, Bacillus subtilis, Bacillus megaterium, Neisseria lactamica, Salmonella, Klebsiella, Pseudomonas, Caulobacter, Rhizobium and the like. Other bacteria of interest are disclosed in the following publications: Ferre-Miralles et al, 2013, Microbial Cell Factories, 12, 113; Pharm et al, 2019, Front. Microbiol., 10, Article 1404; Weinstock et al, 2016, Nature Methods, 13, 849-851; Vos et al, 2009, The ISME Journal, 3, 199-208).

In preferred aspects, the bacterial cell is a competent bacterial cell, preferably a competent bacterial cell suitable for transformation with a vector or set of vectors comprising elements of the modules 1, 2, 3 or 4. In a more preferred aspect, the competent bacterial cell provides an optimal level of expression from a low number of copies. Competent strains that provides such an advantageous feature are well known to the person skilled in the art, especially among Escherichia coli strains. For instance, the competent bacterial cell is derived from the BL21(DE3) strain, DH10B, Marionette Clo (Addgene Ref #108251), in particular with the removal of a chloramphenicol resistance gene (coding for chloramphenicol resistance protein, SEQ ID NO: 32), or Acella™ (Zageno, Ref #36795).

In a particular aspect, the bacterium has a genotype F-ompT hsdSB(rB-mB-) gal dcm (DE3) ΔendA ΔrecA such as Acella™, a genotype F-ompT hsdSB (rB-, mB-) gal dcmrne131 (DE3) such as BL21(DE3) Star cells, or a genotype F-mcrA Δ(mrr-hsdRMS-mcrBC) Φ80dlacZΔM15 ΔlacX74 endA1 recA1 deoR Δ(ara,leu)7697 araD139 galU galK nupG rpsL λ-Marionette(Δ CmR) such as a strain derived from Marionette Clo, or MG1655 (ybhB-bioAB)::[lcI857 N(cro-ea59)] tetA recJ⁻ sbcB⁻ ΔaraBAD ΔmutS such as strain bMS_453 (kindly provided by Church Lab, Harvard, MIT).

In preferred aspect, the bacterial cell has an improved plasmid stability. In another preferred aspect, the bacterial cell has a reduced endogenous recombination. In a more preferred aspect, the bacterial cell has both an improved plasmid stability and a reduced endogenous recombination. In preferred aspects, the bacterial cell has an increased proliferation rate.

The stability of oligonucleotides in the bacterial cell can be increased by means referred as preservative effectors. Different types of preservative effectors can be used and optionally combined according to the second module, such as effectors impairing the function of the MMR system or effectors increasing RNA or DNA stability in the bacterial cell.

The present invention relates to a bacterial cell at least one preservative effector in any aspect or combinations thereof and the use thereof for generating diversity in a gene of interest and for increasing the stability of oligonucleotides in the bacterial cell, thereby improving the generation of diversity in a gene L.

Optionally, the bacterial cell has a constitutive or inducible modification improving RNA stability. In the bacterial cell, the RNA stability is important to ensure the formation of retrotranscribing complexes, such as RTC. Preferably, the improved RNA stability of the bacterial cell is due to a reduced RNAse activity while sustaining normal growth of the bacterial cell. More preferably, the reduced RNAse activity of the bacterial cell is due to mutations on at least one RNAse gene, such as rne, pnp, or rnr, that respectively encode the RNAse E, the PnPase and the RNase R (Ikeda et al, 2011, Molecular Microbiology, 79, 419-432; Lopez et al, 1999, Molecular Microbiology, 33, 188-199; Bechhofer et al, 2019, Critical Reviews in Biochemistry and Molecular Biology, 54, 242-300). Even more preferably, the mutations on at least one RNAse gene does not alter the normal growth of the bacterial cell. Optionally, the bacterial cell may constitutively express a RNAse E mutant defined by the rne131 mutation.

The present invention relates to a method for generating diversity in a gene L, wherein the bacterial cell further comprises at least one preservative effector capable of impairing RNAse activity such as rhlB or a fragment 711-844 of RNAse E, and/or capable of impairing the MMR function such as dam, and/or capable of increasing stability of single strand DNA such as mutant ssDNA exonuclease. Optionally, the preservative effector capable of increasing the RNA stability can be an effector that competes with RNAse E for interaction with the protein Hfq. Indeed, the above-mentioned interaction between RNAse E and the Hfq protein promotes the degradation of Hfq bounds RNAs. So strategies that inhibit this interaction can improve Hfq bound RNAs half-life with beneficial effects on cDNA synthesis by reverse-transcription.

The effector capable of increasing the RNA stability can be an RNA helicase such as rhlB, whose sequence corresponds to SEQ ID NO: 61, or can be a fragment 711-844 of RNAse E (SEQ ID NO: 63) (Ikeda et al, 2011, 79, 419-432). Since rhlB interacts with RNAse E at the same epitope recognized by Hfq, the over-expression of rhlB can inhibit the interaction between Hfq and RNAse E by competition.

Alternatively, the effector capable of increasing the RNA stability can be the fragment (711-844) of RNAse E. The binding of the RNAse (711-844) peptide to the Hfq protein thus prevents it to interact with the whole functional RNAse E that includes the N-terminal catalytic region.

Then, the bacterial cell may express constitutively or inductively an RNA helicase such as rhlB or a fragment 711-844 of RNAse E as detailed above.

In yet another aspect, alternative or additional, the preservative effector can be an effector that increases the ssDNA strands stability.

Optionally, the bacterial cell has a constitutive or inducible modification reducing linear DNA degradation. Preferably, the reduced linear DNA degradation of the bacterial cell is due to a reduced ssDNAse and/or dsDNAse activity of the bacterial cell. More preferably, the reduced DNAse activity of the bacterial cell is due to mutations on at least one ssDNA exonuclease gene, such as xonA, recJ, xseA exoX. In particular, the mutant ssDNA exonuclease whose exonuclease function is reduced or invalidated can be a mutant xonA (such as SEQ ID NO: 64), a mutant xseA (such as SEQ ID NO: 66), a mutant exoX (such as SEQ ID NO: 65), or a mutant recJ (such as SEQ ID NO: 67) (Mosberg et al, 2012, PLOS One, 7, e44638; Gallagher et al, 2014, Nature Protocols, 9, 2301-2316; Dutra et al, 2007, PNAS, 104, 216-221; Simon et al, 2018, ACS Synth Biol, 7, 2600-2611). Generally, the invalidated gene is generated by knockout or by introduction of a STOP codon in the coding sequence and/or by introducing a change in the open reading frame.

The preservative effector can be an effector capable of impairing the function of the MMR system. Optionally, the bacterial cell has a constitutive or inducible modification impairing the MMR system. Preferably, the impairment of the MMR system of the bacterial cell is due to mutations on MMR component genes, such as mutL, mutS, mutH or UvrD, in particular a dominant mutant of MutS, a dominant mutant of MutL or a dominant mutant of MutH (Junop et al, 2003, DNA Repair, 2, 387-405; Yang et al, 2004, Molecular Microbiology, 53, 283-295). Alternatively or in addition, the impairment of the MMR system of the bacterial cell can be caused by the over-expression of the DNA methylase such as dam. Indeed, the over expression of Dam can increase DNA methylation and impair the recognition of neosynthesized cDNA copies of gene L during mismatch repair. Since the decrease in MMR function should also result in higher levels of mutations over non-target sites, preservative effectors that impairs the MMR function are preferably over-expressed by transient methods in the bacterial cell. In particular aspects, the bacterial cell belongs to Nuc5-, EcNR3, or EcM2.1 strains (Gallagher et al, 2014, Nat. Protoc., 9, 2301-2316) or TOP10 dXseA/dMutS strain (Simon, Morrow and Ellington, 2018, ACS Synth. Biol., acssynbio.8b00273). Nuclease invalidated strain can be found among George Church Lab's strains available at Addgene: addgene.org/search/catalog/bacterial-strains/?q=george+church.

In preferred aspects, the bacterial cell is capable of over-expressing recombinase, in particular a beta recombinase such as lambda phage recombination factors, in particular in an inducible way, for instance when the temperature is shifted above 37° C. An example of such a bacterial cell is DY380 strain. Alternative recobineering strains, including DY380, can be found at Court lab recombineering website (https://redrecombineering.ncifcrf.gov).

Accordingly, the bacterial cell may have one or more of the following features: constitutive or inducible improvement in RNA stability, decrease of linear DNA degradation, impairment of the DNA mismatch repair system, and increased proliferation.

### Combinations of Modules

The present invention relates to the combination of modules 1 and 2, preferably with the co-localization strategy, modules 1, 2 and 3, optionally with the co-localization strategy, and modules 1, 2, 3 and 4, optionally with the co-localization strategy.

Therefore, it relates to bacterial cells and/or vectors or set of vectors comprising the elements of these modules as disclosed above. Optionally, all the element can be comprised into the bacterial cells. Optionally, some of the elements can be comprised into the bacterial cells and the others on vectors or set of vectors. Optionally, all the element can be comprised on vectors or set of vectors. The present invention relates to the use of these bacterial cells and/or vectors or set of vectors for generating diversity and selecting variants.

The bacterial cells and/or vector or set of vectors can be provided as a kit for generating diversity and selecting variants. The present invention relates to this kit, and the use thereof for generating diversity and selecting variants.

The present invention also relates to a vector or set of vectors comprising the elements as defined below and a bacterial cell comprising this vector or set of vectors or comprising the elements as defined below, the elements being:
a transcription cassette (tC1) comprising a sequence encoding a tpRNA operably linked to a promoter (P1), said tpRNA comprising from 5′ to 3′: a gene L, an RTtag sequence operably linked to the gene L and a SPBM1 sequence, wherein said tC1 is suitable for allowing, in the bacterial cell, the transcription of a tpRNA, wherein the SPBM1 is capable of binding to an SP present in the bacterial cell at a first specific binding site (SPS1);
a transcription cassette (tC2) comprising a sequence encoding a prRNA operably linked to a promoter (P2), said prRNA comprising: an RBM sequence positioned in 5′ end, preferably the RBM of SEQ ID NO: 7, an SPBM2 sequence, preferably the SPBM2 of SEQ ID NO: 18, and an RTprimer, preferably an RTprimer of SEQ ID NO: 13, wherein said tC2 is suitable for allowing, in the bacterial cell, the transcription of a prRNA, wherein the RTprimer is capable of complementary pairing to the RTtag, the SPBM2 is capable of binding to the SP, the sequence encoding the prRNA optionally further comprising a sequence encoding a tRNA sequence contiguously positioned downstream of the RTprimer sequence, a site cleavable by an RNAse of the bacterial cell is present between said tRNA sequence and said RTprimer, thereby allowing the production of a well-defined 3′ prRNA end;
an expression cassette (eC1) comprising a sequence encoding an RBD-RT fusion protein operably linked to a promoter (P3), said RBD-RT comprising a reverse transcriptase (RT) sequence, especially TF1 RT (e.g., of SEQ ID NO: 1), MMLV RT (e.g., SEQ ID NO: 3) or HIV-1 RT (e.g., of SEQ ID NO: 2), and an RBD sequence, preferably an RBD of SEQ ID NO: 5, wherein said eC1 is suitable for allowing, in the bacterial cell, the expression of the RBD-RT fusion protein, wherein the RBD is capable of binding to the RBM of prRNA,
an expression cassette (eC2) comprising a sequence encoding the SP operably linked to a promoter (P4), preferably said SP being the Hfq protein, preferably the Hfq of SEQ ID NO: 15, wherein eC2 is suitable for allowing, in the bacterial cell, the expression of the SP, preferably the Hfq protein, and
an expression cassette (eC3) comprising an HR gene operably linked to a promoter (P5), wherein said eC3 is suitable for allowing, in the bacterial cell, the expression of an HR capable of integrating the altered copies of the gene L into a DNA vector or into the genome of the bacterial cell, said vector or genome comprising a copy of the gene L, thereby preserving the altered copies of the gene L from degradation.

Optionally, the vector or set of vectors or the bacterial cell comprising this vector or set of vectors further comprises:
an expression cassette (eC4) comprising a sequence encoding a reporter gene operably linked to a promoter (P6),
an expression cassette (eC5) comprising a sequence encoding an FPR protein operably linked to a promoter (P7), said FPR comprising a target domain and a DBD sequence, said DBD being capable of binding to a site located at proximity of the promoter P6 so as to promote the expression of the reporter gene when the target molecule is bound to a variant encoded by an altered copy of the gene L, wherein said eC5 is suitable for allowing, in the bacterial cell, the expression of an FPR protein, and
an expression cassette (eC6) comprising a sequence encoding an FPL protein operably linked to a promoter (P8), said FPL comprising an insertion site suitable for the insertion of the gene L and transcription subunits (TrSu) capable of recruiting an RNA polymerase, wherein said eC6 is suitable for allowing, in the bacterial cell, the expression of a FPL protein comprising either a ligand encoded by the gene L or a variant thereof encoded by an HR-integrated altered copy of gene L;

or
an expression cassette (eC4) comprising a sequence encoding a reporter gene operably linked to a promoter (P6),
an expression cassette (eC5) comprising a sequence encoding an FPR protein operably linked to a promoter (P7), said FPR comprising a target domain and transcription subunits (TrSu) capable of recruiting an RNA polymerase, wherein said eC5 is suitable for allowing, in the bacterial cell, the expression of an FPR protein, and
an expression cassette (eC6) comprising a sequence encoding an FPL protein operably linked to a promoter (P8), said FPL comprising an insertion site suitable for the insertion of the gene L and a DBD sequence, said DBD being capable of binding to a site located at proximity of the promoter P6 so as to promote the expression of the reporter gene when the target molecule is bound to a variant encoded by an altered copy of the gene L, wherein said eC6 is suitable for allowing, in the bacterial cell, the expression of an FPL protein comprising either a ligand encoded by the gene L or a variant thereof encoded by an HR-integrated altered copy of gene L;

or
an expression cassette (eC4) comprising a sequence encoding a reporter gene operably linked to a promoter (P6),
an expression cassette (eC5) comprising a sequence encoding an FPR protein operably linked to a promoter (P7), said FPR comprising a target domain and a DBD sequence, said DBD being capable of binding to a site located at proximity of the promoter P6 so as to promote the expression of the reporter gene when the target molecule is bound to a variant encoded by an altered copy of the gene L, wherein said eC5 is suitable for allowing, in the bacterial cell, the expression of an FPR protein, and
an expression cassette (eC6) comprising a sequence encoding an FPL protein operably linked to a promoter (P8), said FPL comprising the gene L and transcription subunits (TrSu) capable of recruiting an RNA polymerase, wherein said eC6 is suitable for allowing, in the bacterial cell, the expression of a FPL protein comprising either a ligand encoded by the gene L or a variant thereof encoded by an HR-integrated altered copy of gene L;

or
an expression cassette (eC4) comprising a sequence encoding a reporter gene operably linked to a promoter (P6),
an expression cassette (eC5) comprising a sequence encoding an FPR protein operably linked to a promoter (P7), said FPR comprising a target domain and transcription subunits (TrSu) capable of recruiting an RNA polymerase, wherein said eC5 is suitable for allowing, in the bacterial cell, the expression of an FPR protein, and
an expression cassette (eC6) comprising a sequence encoding an FPL protein operably linked to a promoter (P8), said FPL comprising the gene L and a DBD sequence, said DBD being capable of binding to a site located at proximity of the promoter P6 so as to promote the expression of the reporter gene when the target molecule is bound to a variant encoded by an altered copy of the gene L, wherein said eC6 is suitable for allowing, in the bacterial cell, the expression of an FPL protein comprising either a ligand encoded by the gene L or a variant thereof encoded by an HR-integrated altered copy of gene L.

Optionally, the vector or set of vectors or the bacterial cells further comprises: a sequence encoding a DNA invertase gene operably linked to P6 in the eC4 expression cassette; and DNA invertase sites flanking the sequence encoding the RT and/or the HR, respectively in the eC1 and eC3 expression cassettes.

Optionally, the vector or set of vectors or the bacterial cells further present the following features: the eC1 further comprises restriction sites flanking the sequence encoding RBD-RT and/or the eC3 further comprises restriction sites flanking the sequence encoding HR factor gene, and the eC4 further comprises a sequence encoding a restriction enzyme gene operably linked to P6.

Optionally, the vector or set of vectors or the bacterial cells further present the following features: the eC4 further comprises a sequence encoding a transcription repressor gene operably linked to P6, and the expression of the sequence encoding RBD-RT of the eC1 and/or the sequence encoding HR factor gene of the eC3 can be stopped by said transcription repressor gene.

The present invention further relates to a vector or set of vectors, said vector or set of vectors comprising:
a transcription cassette (tC1) comprising a sequence encoding a pre-tpRNA operably linked to a promoter (P1), said pre-tpRNA comprising from 5′ to 3′: an insertion site suitable for the insertion of a gene L, an RTtag sequence, preferably an RTtag of SEQ ID NO: 14, operably linked to the gene L to be inserted and a SPBM1 sequence, preferably a SPBM1 of SEQ ID NO: 17, wherein said tC1 is suitable for allowing, in the bacterial cell, the transcription of a tpRNA including an inserted gene L, wherein the SPBM1 is capable of binding to an SP present in the bacterial cell at a first specific binding site (SPS1);
a transcription cassette (tC2) comprising a sequence encoding a prRNA operably linked to a promoter (P2), said prRNA comprising: an RBM sequence positioned in 5′ end, preferably the RBM of SEQ ID NO: 7, an SPBM2 sequence, preferably the SPBM2 of SEQ ID NO: 18, and an RTprimer, preferably an RTprimer of SEQ ID NO: 13, wherein said tC2 is suitable for allowing, in the bacterial cell, the transcription of a prRNA, wherein the RTprimer is capable of complementary pairing to the RTtag, the SPBM2 is capable of binding to the SP, the sequence encoding the prRNA optionally further comprising a sequence encoding a tRNA sequence contiguously positioned downstream of the RTprimer sequence, a site cleavable by an RNAse of the bacterial cell is present between said tRNA sequence and said RTprimer, thereby allowing the production of a well-defined 3′ prRNA end;
an expression cassette (eC1) comprising a sequence encoding an RBD-RT fusion protein operably linked to a promoter (P3), said RBD-RT comprising a reverse transcriptase (RT) sequence, especially TF1 RT (e.g., of SEQ ID NO: 1), MMLV RT (e.g., SEQ ID NO: 3) or HIV-1 RT (e.g., of SEQ ID NO: 2), and an RBD sequence, preferably an RBD of SEQ ID NO: 5, wherein said eC1 is suitable for allowing, in the bacterial cell, the expression of the RBD-RT fusion protein, wherein the RBD is capable of binding to the RBM of prRNA,
optionally, an expression cassette (eC2) comprising a sequence encoding the SP operably linked to a promoter (P4), preferably said SP being the Hfq protein, preferably the Hfq of SEQ ID NO: 15, wherein eC2 is suitable for allowing, in the bacterial cell, the expression of the SP, preferably the Hfq protein, and
an expression cassette (eC3) comprising an HR gene operably linked to a promoter (P5), wherein said eC3 is suitable for allowing, in the bacterial cell, the expression of an HR capable of integrating the altered copies of the gene L into a DNA vector or into the genome of the bacterial cell, said vector or genome comprising a copy of the gene L, thereby preserving the altered copies of the gene L from degradation.

Optionally, the vector or set of vectors further comprises:
an expression cassette (eC4) comprising a sequence encoding a repressor gene operably linked to a promoter (P6),
an expression cassette (eC4′) comprising a sequence encoding a reporter gene operably linked to a promoter (P6′), the expression of the reporter gene being negatively controlled by the repressor encoded by (eC4),
an expression cassette (eC5) comprising a sequence encoding an FPR protein operably linked to a promoter (P7), said FPR comprising a target domain and a DBD sequence, said DBD being capable of binding to a site located at proximity of the promoter P6 so as to promote the expression of the repressor gene when the target molecule is bound to a variant encoded by an altered copy of the gene L, wherein said eC5 is suitable for allowing, in the bacterial cell, the expression of an FPR protein, and
an expression cassette (eC6) comprising a sequence encoding an FPL protein operably linked to a promoter (P8), said FPL comprising an insertion site suitable for the insertion of the gene L and transcription subunits (TrSu) capable of recruiting an RNA polymerase, wherein said eC6 is suitable for allowing, in the bacterial cell, the expression of a FPL protein comprising either a ligand encoded by the gene L or a variant thereof encoded by an HR-integrated altered copy of gene L;

or
an expression cassette (eC4) comprising a sequence encoding a repressor gene operably linked to a promoter (P6),
an expression cassette (eC4′) comprising a sequence encoding a reporter gene operably linked to a promoter (P6′), the expression of the reporter gene being negatively controlled by the repressor encoded by (eC4),
an expression cassette (eC5) comprising a sequence encoding an FPR protein operably linked to a promoter (P7), said FPR comprising a target domain and transcription subunits (TrSu) capable of recruiting an RNA polymerase, wherein said eC5 is suitable for allowing, in the bacterial cell, the expression of an FPR protein, and
an expression cassette (eC6) comprising a sequence encoding an FPL protein operably linked to a promoter (P8), said FPL comprising an insertion site suitable for the insertion of the gene L and a DBD sequence, said DBD being capable of binding to a site located at proximity of the promoter P6 so as to promote the expression of the repressor gene when the target molecule is bound to a variant encoded by an altered copy of the gene L, wherein said eC6 is suitable for allowing, in the bacterial cell, the expression of an FPL protein comprising either a ligand encoded by the gene L or a variant thereof encoded by an HR-integrated altered copy of gene L;

or
an expression cassette (eC4) comprising a sequence encoding a repressor gene operably linked to a promoter (P6),
an expression cassette (eC4′) comprising a sequence encoding a reporter gene operably linked to a promoter (P6′), the expression of the reporter gene being negatively controlled by the repressor encoded by (eC4),
an expression cassette (eC5) comprising a sequence encoding an FPR protein operably linked to a promoter (P7), said FPR comprising a target domain and a DBD sequence, said DBD being capable of binding to a site located at proximity of the promoter P6 so as to promote the expression of the repressor gene when the target molecule is bound to a variant encoded by an altered copy of the gene L, wherein said eC5 is suitable for allowing, in the bacterial cell, the expression of an FPR protein, and
an expression cassette (eC6) comprising a sequence encoding an FPL protein operably linked to a promoter (P8), said FPL comprising the gene L and transcription subunits (TrSu) capable of recruiting an RNA polymerase, wherein said eC6 is suitable for allowing, in the bacterial cell, the expression of a FPL protein comprising either a ligand encoded by the gene L or a variant thereof encoded by an HR-integrated altered copy of gene L;

or
an expression cassette (eC4) comprising a sequence encoding a repressor gene operably linked to a promoter (P6),
an expression cassette (eC4′) comprising a sequence encoding a reporter gene operably linked to a promoter (P6′), the expression of the reporter gene being negatively controlled by the repressor encoded by (eC4),
an expression cassette (eC5) comprising a sequence encoding an FPR protein operably linked to a promoter (P7), said FPR comprising a target domain and transcription subunits (TrSu) capable of recruiting an RNA polymerase, wherein said eC5 is suitable for allowing, in the bacterial cell, the expression of an FPR protein, and
an expression cassette (eC6) comprising a sequence encoding an FPL protein operably linked to a promoter (P8), said FPL comprising the gene L and a DBD sequence, said DBD being capable of binding to a site located at proximity of the promoter P6 so as to promote the expression of the repressor gene when the target molecule is bound to a variant encoded by an altered copy of the gene L, wherein said eC6 is suitable for allowing, in the bacterial cell, the expression of an FPL protein comprising either a ligand encoded by the gene L or a variant thereof encoded by an HR-integrated altered copy of gene L.

Optionally, the vector or set of vectors further comprises: a sequence encoding a DNA invertase gene operably linked to P6 in the eC4 expression cassette; and DNA invertase sites flanking the sequence encoding the RT and/or the HR, respectively in the eC1 and eC3 expression cassettes.

Optionally, the vector or set of vectors or the bacterial cells further present the following features: the eC1 further comprises restriction sites flanking the sequence encoding RBD-RT and/or the eC3 further comprises restriction sites flanking the sequence encoding HR factor gene, and the eC4 further comprises a sequence encoding a restriction enzyme gene operably linked to P6.

Optionally, the vector or set of vectors or the bacterial cells further present the following features: the eC4 further comprises a sequence encoding a transcription repressor gene operably linked to P6, and the expression of the sequence encoding RBD-RT of the eC1 and/or the sequence encoding HR factor gene of the eC3 can be stopped by said transcription repressor gene.

Optionally, some encoding sequences can be arranged in a polycistronic constructs and their expression can be controlled by the same promoter. For instance, the RT, especially RBD-RT and the HR can be assembled as a bicistronic construct and their expression can be controlled by the same promoter. The FPL and FPR coding region can also constitute bicistronic constructs controlled by the same promoter. Finally, bi- or polycistronic constructions can be used for generating signals correlated to the interaction between FPL and FPR. Preferentially, fluorescent or luminescent proteins can be coupled to antibiotic resistance markers and/or genes related to the system arrest such as DNA invertases, restriction enzymes or repressors.

### EXAMPLES

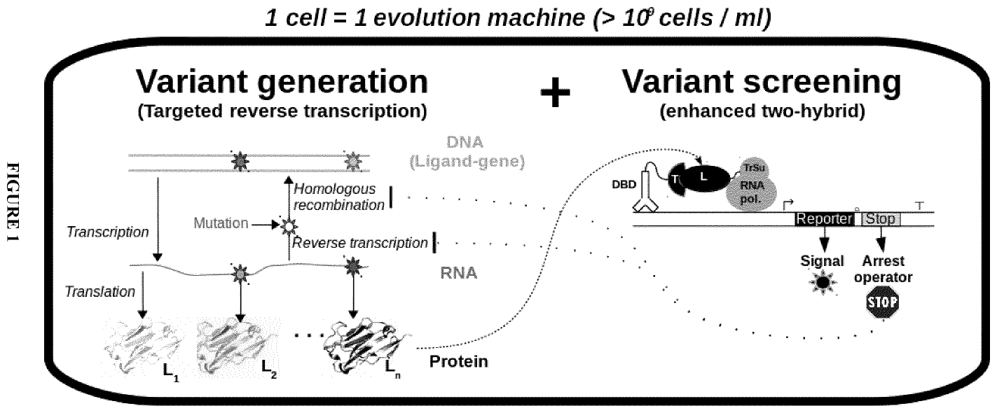

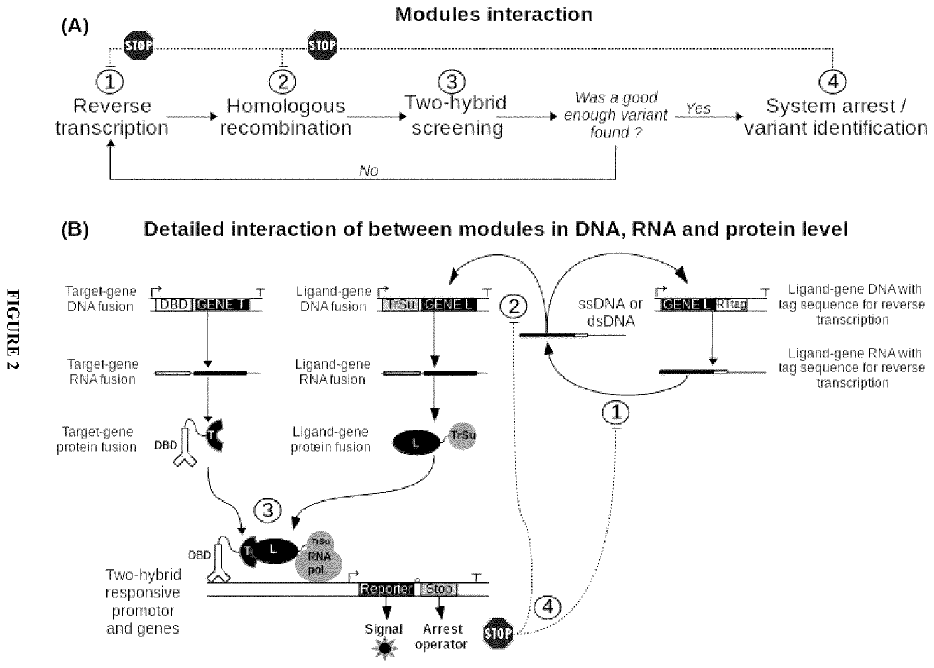

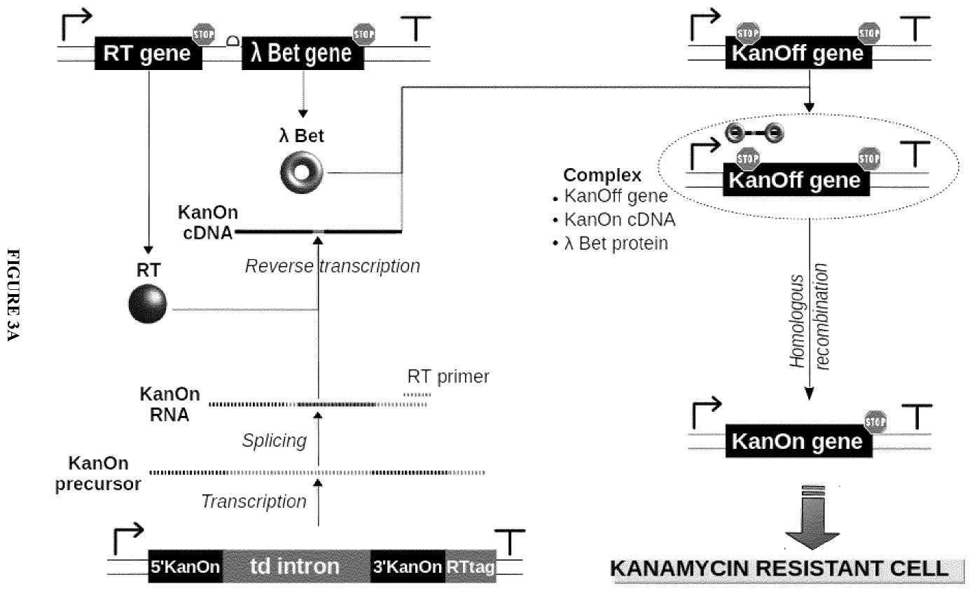

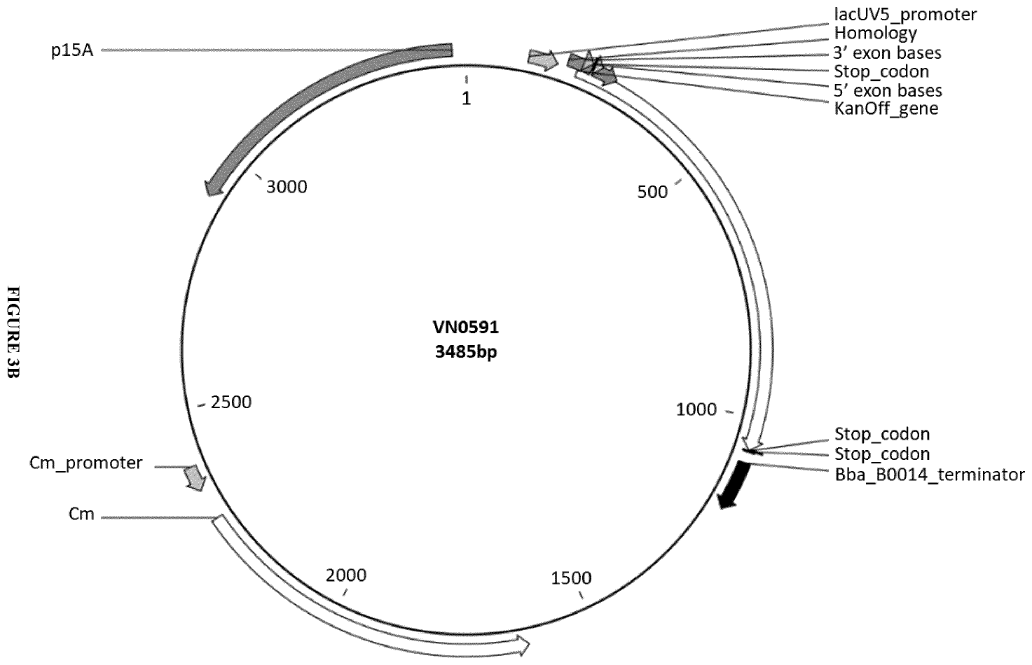

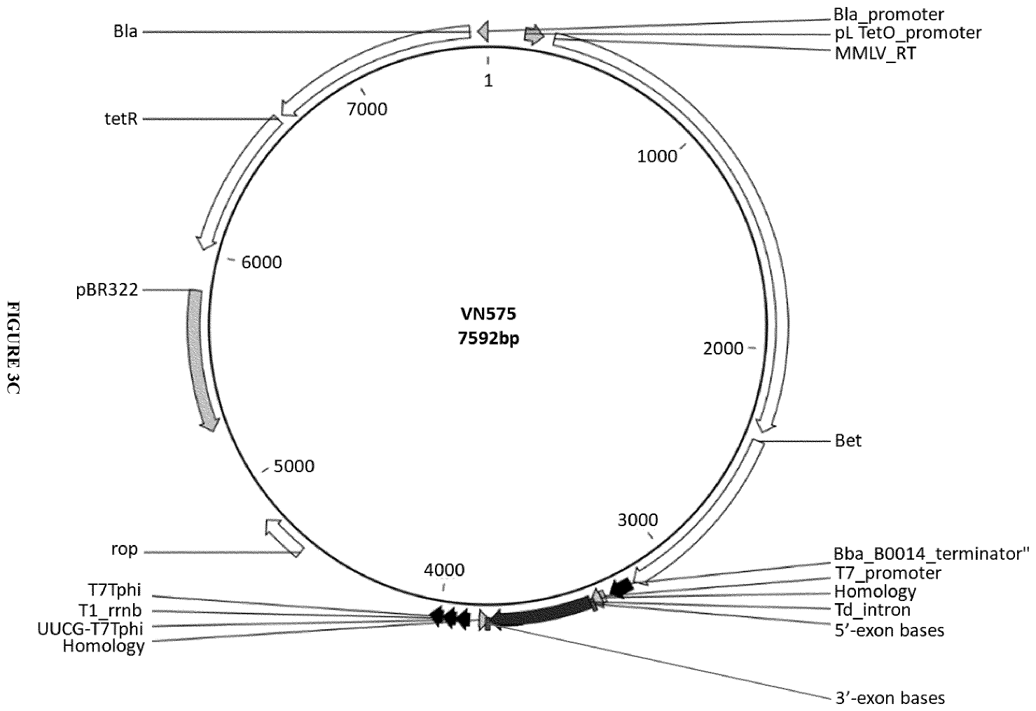

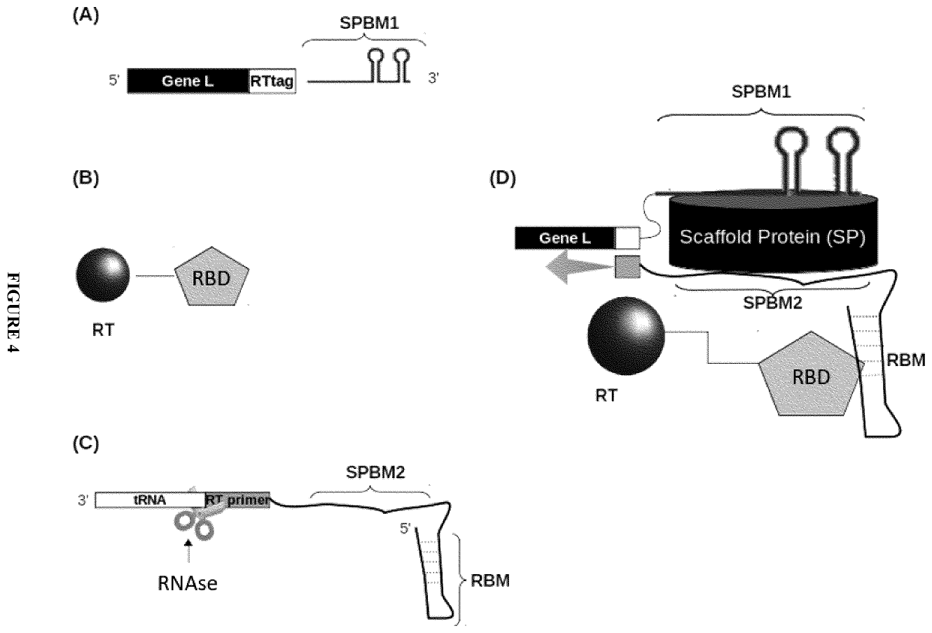

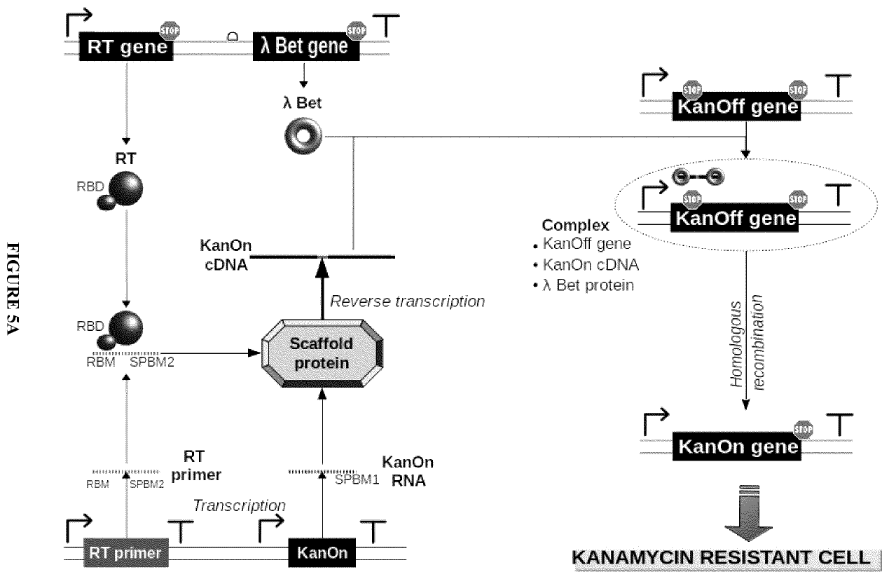

Due to the complexity of the 4-module system, the inventors began implementing the modules and testing them in pairs before implementing a complete 4-module system. The four module system is schematically disclosed in FIGS. 1 and 2.

### Example 1: Test of RT/HR Coupling

In order to test the coupling of RT (reverse transcription) and HR (homologous recombination) modules in a bacterial cell, an artificial biological system implemented in two plasmids was constructed (FIG. 3A). The first plasmid (FIG. 3B, VN591; SEQ ID NO: 37) harbors a modified kanamycin resistance gene with an internal stop codon. Consequently, a truncated (not functional) protein is produced and does not grant kanamycin resistance to transformed bacterial cells (KanOff gene). The second plasmid (FIG. 3C, VN575; SEQ ID NO: 38) provides a coding region of a retroviral reverse transcriptase (“MMLV_RT” corresponding to SEQ ID NO: 3, also referred as RT) and a lambda phage recombination factor (“Bet”, also referred as λBet), both comprised in a bicistronic construct. The same plasmid also contains a region which encompasses the following segments: a) a segment homologous to the KanOff gene of the first plasmid (VN591) immediately downstream of the stop codon, followed by; b) a group I intron capable of spontaneous self-splicing from RNA in bacterial cells (td intron); c) a segment homologous to the KanOff gene, immediately upstream of the stop codon and; d) a sequence corresponding to the reverse complement (RTtag) of RNA oligonucleotide (for instance an endogenous small RNA (sRNA) or a designed transcript) that should function as primer for reverse transcription (RT primer).

As illustrated in FIG. 3A (bottom-left part), the transcription of this region generates an RNA including an internal intron (KanOn precursor). The intron region self-splices, giving rise to an intronless RNA product (KanOn RNA) corresponding to the fusion of the homologous regions present at the extremities of the unprocessed RNA plus the RTtag. The intronless transcript and RT primer then hybridize by their complementary regions and the RT enzyme synthesizes a complementary DNA strand (KanOn cDNA) complementary to the flanking regions of the internal stop codon present at the KanOff gene. Thus, the free 3′OH of RT primer is used for DNA polymerization and the KanOn RNA is used as a template. When both VN591 and VN575 plasmids co-transform bacterial cells, the KanOn cDNA produced is used by λBet protein for homologous recombination and the outcome should be the deletion of the internal stop codon on the KanOff gene and, consequently, the rescue of kanamycin resistance. Therefore, only if KanOn cDNA is generated from intronless RNA molecules by reverse transcription and recombines with KanOff gene the corresponding cells can proliferate in the presence of kanamycin, because recombination products involving exclusively the plasmids do not rescue kanamycin resistance. Indeed, the alternative possibility of direct recombination involving DNA from the plasmids would not rescue kanamycin resistance because the intron sequence (about 437 base pairs) generates insertions, several stop codons and a frame-shift that would surely abrogate the expression of a functional kanamycin resistance gene.

To test this hypothesis, Acella cells, a BL21(DE3) derived strain that provides better plasmid stability and reduces non lambda factors mediated recombination, were co-transformed with VN575 and VN591 (plasmids described in FIGS. 3B and 3C) and cultivated overnight (37° C., 200 rpm) in SOB medium supplemented with antibiotics (75 μg/ml Ampicillin, 25 μg/ml Chloramphenicol). Next, the saturated culture was diluted (1:100) and induced (aTc 100 ng/ml; IPTG 100 μM). After about 3 hours incubation (37° C., 200 rpm), the cells (O.D.=0.5-0.6) were plated in LB-Agar plates containing kanamycin (1 mg/ml) and IPTG (100 μM) for colony counting and sequencing. The kanamycin resistant clones contained exactly the expected final sequence (without stop codon) and no colony was found containing the intron region. Thus, the feasibility of the coupling between RT and RH modules was considered validated. It is the first time that the feasibility of the reverse transcription using a retroviral reverse transcriptase and no tRNA derived primer structure (or 3′ self-primer strategy) as primer is demonstrated in E. coli, therefore, unleashing the intracellular use of these enzymes from this requirement.

The efficiency of the coupling between RT and HR (evaluated by the frequency of selected kanamycin resistant clones) should rely on several steps and factors including: a) the expression levels of RT and Bet; b) the transcription level of the intron containing RNA and its self-splicing efficiency; c) the concentration of intracellular oligonucleotides that should function as primer for reverse transcription; d) the secondary structure stability of each RNA involved and their half-life; d) recognition of dsRNA stretches by the RT and the efficiency of cDNA synthesis; e) degradation of RNA strand of the DNA/RNA hybrid; f) the rate of cDNA degradation by intracellular single-strand exonucleases (such as xonA, xseA, exoX and recJ) and; g) Bet (or other annealing protein) promoted recombination of the synthesized cDNA (KanOn cDNA) with the target plasmid (KanOff gene).

In the assays using intron containing RNAs, the observed frequency (counted colonies/total plated cells) of kanamycin resistant colonies was about 4.02×10⁻⁹ (FIG. 10, (1)), demonstrating that system works but with a low efficiency. Some possible explanations for this can be related to: a) the presence of the intron that does not self-splices efficiently in the current context; b) structural elements of the involved RNAs can be stable enough to impairs double-stranded RNA (dsRNA) annealing and, subsequent, reverse transcription; c) the fast turnover of RNAs in Escherichia coli (Selinger and cols, 2003, found that half-life of total mRNA was about 6.8 minutes and some mRNA have half-life ≤2.5 minutes) reduces the probability of reverse transcription complex formation (RNA template, RNA primer and reverse transcriptase); d) insufficient amounts of annealing protein (λBet monomer) is expressed (from the bicistronic RNA including RT enzyme), thereby impairing the formation of λBet functional multimers.

### Example 2: Implementation of the Co-Localization Strategy

In order to address some of the above-mentioned potential problems, a new system was designed to recruit the kanON RNA, RNA primer and RT enzyme on a scaffold in order to increase involved RNA half-life, to promote dsRNA annealing, to increase local concentration of the ternary complex members (RT template, RT primer and RT enzyme) and, consequently, to improve the likelihood of cDNA synthesis. The selected scaffold was the Hfq protein. Thoughtfully, in order to this recruitment strategy to work, specific RNA secondary structures are required. Thus, the RNA involved in the complex comprise specific RNA regions either dedicated to interact with the protein scaffold (in some embodiment SPBM1 and SPBM2) or RT interactions (in some embodiment, RBM) (FIG. 4).

One implementation of this new strategy was tested using DY380 cells that over-expresses lambda recombination factors when the temperature is shifted above 37° C. Cells were co-transformed with KanOff plasmid (FIG. 3B, VN591) and the new KanOn plasmid (FIG. 5B, VN669; SEQ ID NO: 39) and cultivated overnight (30° C., 200 rpm) in SOC medium supplemented with antibiotics (75 μg/ml Ampicillin, 25 μg/ml Chloramphenicol). Next, the saturated culture was diluted (1:65), incubated (30° C., 200 rpm) for 2 hours (O.D.>0.1) and induced (aTc 100 ng/ml; IPTG 100 μM, 42° C. for 12 minutes). After about 2 hours, cells were plated in LB-Agar plates containing kanamycin (1 mg/ml) and IPTG (100 μM) for colony counting and sequencing. Surprisingly, this new strategy resulted in an improved frequency of 3.12×10⁻⁶ (FIG. 10, (2)), more than 750 times more efficient than the former system including td intron and no recruitment of RNAs and RT enzyme (example 1). The sequencing results indicate that DNA products correspond exactly to the expected sequence.

Also, the strategy concerning the generation of RT primer could be applied to the intracellular generation of RNAs with defined sequence at 3′. The latter strategy consists in fusing an RNA region to a tRNA containing a leader sequence that should be split off by a host cell RNAse, such as RNAse P (FIG. 4C).

### Example 3: Adaptation of the System to Ligand Screening Using an Enhanced Bacterial Two Hybrid (B2H) System

Concerning the third module (eB2H), first, the inventors have tested currently available B2Hs (bacterial two-hybrid systems), such as the one created by the team of Ann Hochschild (Harvard University, USA; Nickels, 2009) and Rama Ranganathan (Green Center for Systems Biology, USA; McLaughlin, 2012). In order to compare them, the original systems were modified in order to harmonize the plasmids used: the reporter gene (eGFP, SEQ ID NO: 33) and the complex formation partners (FPL and FPR), thus, the only relevant element differing was the two-hybrid responsive promoter. Protein-protein interactions (PPIs) with varying strengths, ranging from 3 to 8000 nM, were tested to evaluate their signal intensities and their correlation to the affinities. Based on the results (FIG. 6A), the inventors have noticed that the former does not provide a sufficient strong signal output and the second does not show good correlations between ligand affinities and signal intensity. Therefore, the inventors had to create by rational genetic engineering a new two-hybrid responsive promoter (FIG. 6B) that conciliates stronger genetic output and improved correlation between affinity with signal intensity. One promoter variant (epB2H, SEQ ID NO: 24) was chosen after comparing some alternatives and the enhanced B2H (eB2H) can provide significant signals even for μM affinities and robust signal for nM affinities. Moreover, this responsive promoter correlates well the complex affinity and the signal output (FIG. 6A).

The tests were carried out by co-transforming BL21(DE3) Star cells with plasmids harboring each of the promoter variants (respectively, VN520, VN552 and VN550 corresponding to SEQ ID NOs: 40-42) and the target gene fused to X. cI DNA binding domain (cI-Asf1) plus one of the plasmids containing different rpoA-peptide fusions (rpoA, RNA polymerase alpha subunit). Each peptide interacts with Asf1 with varying affinities (VN515_IP1: 8000 nM, VN516_IP2: 560 nM, VN517_IP3: 84 nM, VN518_IP4: 3 nM, VN519_IP3mutA: no-interaction; corresponding to SEQ ID NOs: 43-47). Co-transformed cells were cultivated (200 rpm, 37° C., overnight) in LB supplemented with ampicillin (75 μg/ml) and chloramphenicol (25 μg/ml), saturated cultures were diluted 100× and fresh cultures were cultivated for 2h (37° C., 200 rpm). Next, the cultures were induced (20 μM IPTG) and grown overnight (20° C., 200 rpm). The next day, culture samples were diluted in PBS, analyzed by flow cytometry (Millipore Guava easyCyte HT). The mean fluorescence intensity (MFI) of each sample was calculated and plotted against the reported affinity for each peptide binder (FIG. 6A). Thus, it can be noted that, compared to the B2H systems from Hochschild and Ranganathan, the selected responsive promoter correlates well the complex affinity and the signal output (FIG. 6A,“Ramos/martin (2 plasmids)” curve).

The inventors also created a single vector encompassing all biological elements required for the B2H system to work, generated a series of derivatives corresponding to the peptides with varying affinities that the inventors tested under the same conditions but using only chloramphenicol (34 μg/mL) as antibiotic for selection of transformed cells (VN750_IP1: 8000 nM, VN751_IP2: 560 nM, VN752_IP3: 84 nM, VN753_IP4: 3 nM, VN754_IP3mutA: no-interaction; corresponding to SEQ ID NOs: 48-52) (FIG. 6A,“Ramos/martin (1 plasmid)” curve). Interestingly, the single vector configuration allows the B2H system to be more sensitive, with higher MFI values compared to the dual plasmid configuration described above.

Finally, the inventors constructed a series of vectors that indirectly correlate the sensed affinity with the resulting gene expression signal. The signal inversion was obtained by replacing the reporter/marker genes in the previous constructions by a repressor (SrpR) that blocks the transcription from a promoter (T7-SrprOx2) associated to the expression of the reporter/marker genes (FIG. 6A, “Ramos/martin reverse (2 plasmids)” curve). The latter vector series was implemented in a two plasmids setting and tested under the same conditions using ampicillin (75 μg/mL) and chloramphenicol (25 μg/mL). Interestingly, since the fluorescence signals are relatively high in the low affinity range (10³ to 10⁵ nM), the reverse configuration can be particularly interesting for detecting low affinity bindings.

### Example 4: B2H Optimization

In addition to the improved responsive promoter, other modifications of the B2H system were introduced in order to decrease the stochastic behavior.

### 1) cI Fusion Regulation

The expression of the cI fusion element (comprising the DNA binding domain, DBD), was regulated by the promoter lacUV5 (IPTG induced) and its strong RBS in the plasmid VN1197 (SEQ ID NO: 53). In VN1296 (SEQ ID NO: 54), this promoter and its associated RBS were replaced by a strong promoter (pLtetO) associated with a weak RBS. This promoter and this RBS were selected from a library composed of 3 promoters of varying strengths (pLTetO, J23113 and J23116) and 24 RBS variants that have been designed using an RBS Library calculator (https://salislab.net/software/RBSLibraryCalculatorSearchMode, containing RBSs from weak to moderate strength).

Briefly, for promoter+RBS selection, Acella strain was transformed with the library and plated in LB-Agar chloramphenicol containing anhydrotetracycline hydrochloride (aTc, 200 ng/ml) and IPTG (250 μM). The most fluorescent colonies were inoculated in liquid media for plasmid extraction and DNA sequencing. The couple pLTetO+RBS7 was found to be the most prevalent among the combinations that yield high fluorescence.

### 2) The RNA Transcribed as an Output of the Bacterial Two-Hybrid System

In VN1197, it consisted of a tricistronic construction composed of the following elements: RBS+smURFP+RBS+heme oxygenase+weak RBS+kanamycin resistance. In VN1296, the RNA output was replaced by a simpler version composed by the following elements: weak RBS+kanamycin resistance.

### 3) The Strain

VN1197 was tested in Acella while VN1296 was tested in SB33 Strain (having the genome of Marionette Clo (Addgene: 108251) with the removal of the chloramphenicol resistance gene). The genome of SB33 is: F-mcrA Δ(mrr-hsdRMS-mcrBC) Φ80dlacZΔM15 ΔlacX74 endA1 recA1 deoR Δ(ara,leu) 7697 araD139 galU galK nupG rpsL λ-Marionette(ΔCmR).

Then, the inventors tested the effects of the above-mentioned modifications on the stochastic effects by comparing silent mutations of the wild type sequence (FIG. 7). The inventors observed that the use of the strong promoter with a weak RBS allowed a considerable improvement in stochastic effect, by reducing the dispersion of enrichment values.

### Example 5: Adding of a “STOP” Module

To implement the fourth module (diversity generation arrest or “STOP”), a variant of the third module was implemented in a plasmid similar to VN550 (plasmid VN419; SEQ ID NO: 55) in which the two-hybrid responsive promoter controls the transcription of a bicistronic RNA consisting in a DNA invertase gene (BxB1) and a fluorescent reporter gene (eGFP) (FIG. 8A). In the second plasmid (VN376. SEQ ID NO: 56) a DNA region representing the bicistronic RT enzyme+λ Bet protein coded in one strand and a kanamycin resistance gene coded in the reverse complementary strand ( [symbol] ♦RT-Bet=><=KanR⋄⊥) was flanked by DNA invertase sites (“└”, Bxb1 attB and “⋄”, attP). A moderate strength promoter (“ [symbol] ”, Bba_J23105) was placed upstream of this region and the whole fragment was inserted between two different transcription terminators (“ [symbol] ” BBa_B0014 and “⊥” Bba_B0015) (FIG. 8B). As these plasmids also contained DBD-target (cI-PDZ) and TrSu-L fusions (rpoA-L; L=G4S or CRIPT (Cysteine-rich PDZ-binding peptide), Bxb1 DNA invertase should be sufficiently expressed only if the hybrid fusions interact (cI-PDZ+rpoA-CRIPT), thereby inverting the DNA region between Bxb1 attB and attP sites (FIG. 8C). Consequently, RT enzyme and λ Bet should no longer be expressed and the kanamycin resistance gene (coding for kanamycin resistance protein, SEQ ID NO: 34) should now be expressed ( [symbol] >KanR=><=Bet-RT [symbol] ⊥), thus allowing the cells to be selected in presence of kanamycin. The weak RBS (ribosome binding site) controlling Bxb1 translation (FIG. 8A) was selected from a library generated using RBS calculator (Salis, Mirsky & Voigt, 2010) containing 48 variants with predicted strengths between 0.099 and 477.818au. For the convenient selection of RBS strength, the [symbol] ♦RT-Bet=><=KanR⋄⊥ (FIG. 8B) fragment was replaced by [symbol] [symbol] ♦aadA-Bet=><=KanR⋄⊥, and RBSs that do not allow inversion when there is no interaction between hybrid fusions were selected in presence of streptomycin (aaDa encodes for a streptomycin, spectinomycin resistance protein, SEQ ID NO: 35). Then, the sub-library was used to select a new sub-library of RBSs in presence of kanamycin that now allow DNA inversion when the fusion proteins interact ( [symbol] ♦KanR=><=Bet-aadA⋄⊥).

To test if the evolution arrest mechanism worked as expected, BL21(DE3) Star cells (F-ompT hsdSB (rB-, mB-) galdcmrne131 (DE3)) or Acella (F-ompT hsdSB(rB-mB-)gal dcm (DE3) ΔendA ΔrecA, BL21(DE3)) were co-transformed with plasmids VN419 (containing cI-PDZ fusion) and either VN376 or VN405 (respectively: premature stop codon resulting in no fusion peptide or CRIPT fusion peptide; corresponding to SEQ ID NO: 56 and 58) and induced cells (as described for the third module with enhanced B2H) of the corresponding pairs (no-binding: cI-PDZ/rpoA-stop or; 800 nM affinity: cI-PDZ/rpoA-CRIPT) were obtained in LB-Agar supplemented with suitable antibiotics (37° C., overnight). The sequencing results confirm that for colonies representing the non-interacting pair (cI-PDZ/rpoA-stop), the DNA region flanked by Bxb1 attB and attP sites is not inverted, in opposition to colonies representing the interaction cI-PDZ/rpoA-CRIPT.

### Example 6: Whole System Implementation

Since the interactions between the couples of interacting modules (RT and HR; eB2H and STOP) were validated, a new implementation was created to unequivocally and conveniently estimate the efficiency of the whole system (including the four modules in the same cell, represented in FIG. 1 and FIG. 2) by introducing an antibiotic resistance gene (Shble*, invalidated zeocin resistance; Shble coding for a zeocin resistance protein, SEQ ID NO: 36) between the transcription subunit (TrsU, rpoA) and the ligand (SpyTag_D7A, a peptide that interacts with SpyCatcher domain with affinity around 200 nM (Zakeri et al., 2012) in the hybrid construction, therefore creating an extended ligand (Shble*-SpyTag_D7A). Due to the presence of a stop codon in the 5′ region coding the antibiotic resistance sequence (Shble*) that also introduces a frame shift in the downstream open reading frame, the transformed cells are neither resistant to zeocin nor fluorescent (plasmid VN1238, FIG. 9A; corresponding to SEQ ID NO: 59). If the codon is correctly edited in this set up, the corresponding cells become resistant to zeocin and fluorescent because the two-hybrid fusions (cI-SpyCatcher, rpoA-Shble-SpyTag_D7A) should now interact therefore triggering B2H markers and reporters. For convenience, the generation of diversity (RT adapted for co-localization strategy and HR, plasmid VN1228, FIG. 9B; corresponding to SEQ ID NO: 60) and selection of clones related modules (eB2H and STOP, plasmid VN1238, FIG. 9C) were separately implemented, thereby, the whole system is reconstituted (FIG. 9D) by the transformation of cells using both plasmids.

To estimate the frequency of edited cells due to the action of RT and RH modules, the inventors transformed bMS_453 cells with the whole system composed of four modules (plasmids VN1228 and VN1238). Briefly, electrocompetent cells were prepared in room temperature using the protocol described by Tu and cols (2016), transformed cells were recovered in 1 mL SOC media and incubated for 90 minutes. Next, cells were inoculated in 10 mL of LB media supplemented with carbenicillin (75m/mL), chloramphenicol (25 μg/mL), aTc (200 ng/mL), IPTG (20 μM) and incubated overnight. The cultures were diluted (1:200) and incubated for 6 hours; then a dilution corresponding to 500 cells (for the calculations, the concentration of 5×10⁸ cells/mL was considered equivalent to O.D.₆₀₀ₙₘ=1) was plated in LB-agar supplemented with carbenicillin (75 μg/mL), chloramphenicol (25 μg/mL) and IPTG (20 μM) in order to count the number of viable cells. Different amounts of cells (5×10² to 5×10⁶) were plated in LB-agar supplemented with zeocin (30 μg/mL) and IPTG (20 μM) to evaluate the number of edited/evolved cells. All cultures were kept at 31° C. and liquid cultures were shaked at 190 rpm.

The number of viable cells plated in zeocin/IPTG media was corrected based on the proportion of colonies obtained in Carbenicillin/Chloramphenicol media and the frequency of edited/evolved cells was estimated by the ratio between the number of selected cells and the expected number of viable cells that were plated. In opposition to non-edited cells, the majority of selected colonies (zeocin resistant) exhibited intense green fluorescence indicating that the interaction between hybrid proteins was appropriately sensed. Selected colonies were sequenced and the results indicate that the premature stop codon was reverted and the expression of the invertase protein (Bxb1) was sufficient to invert the DNA corresponding to the generation of diversity main effectors (RT+HR) and to activate the expression of the ORF related to Spectinomycin resistance (50 colonies were verified in LB-agar spectinomycin, 50 μg/mL). Furthermore, the analysis of colonies in solid media without zeocin indicated that fluorescent colonies correspond to about 0.75% of the population.

### Example 7: Efficiency Test for Systems with Distinct Modules Implementations

The efficiency of different system implementations expressed as edited cell frequencies is available in FIG. 10. Briefly, cells were transformed with different sets of plasmids allowing different system implementations, cultured under induction prior to be plated in LB-agar containing antibiotics (kanamycin or zeocin) and counted. Some protocols for cell transformation and culture are detailed in previous examples 1 (first bar), 2 (second bar), 4 (third and ninth bar) and 6 (sixth bar).

Interestingly, comparison of the different phenotype frequencies provided by different system implementations allows to highlight the respective benefit of various system modules. Firstly, it can be noted that the use of nuclease mutated strains (for instance bMS_453), even in the presence of the third module (B2H, (3)), significantly increases the phenotype frequency up to 3.08×10⁻⁴, thus indicating an improved generation of diversity compared to the system with only first and second modules implementing the co-localization strategy in cells harboring wild-type nucleases (2). In contrast, this increase in phenotype frequency is less important (5.79×10⁻⁵) for the implementation of the whole system comprising the four modules. This can be explained by an early cessation of the diversity generation process caused by the edition of the stop codon of the Shble* sequence, that allows the expression of a functional ligand (SpyTag_D7A), thereby allowing the expression of the invertase Bxb1 and, consequently, evolution arrest.

In addition, the replacement of the HR (λ Bet) by an RNA helicase (rhlB, (4) and (7)) or a DNA methylase (dam, (6) and (8)) leads to relative decreases in phenotype frequency compared to systems implementing three (3) or four (6) modules. This can be explained by the absence of the HR that significantly reduce the functional coupling between first (RT) and third (B2H) modules, thereby reducing the integration of Shble gene variants into the VN1238 plasmid. However, it is interesting to note that even in absence of HR, the rhlb and dam effectors, coupled with the B2H module, induce a significant improvement of phenotype frequency compared to the “naïve” implementation with no co-localization (1). Nevertheless, the use of effectors alone cannot compensate the absence of HR (respectively, implementations 4 and 5 compared to 3 and; implementations 7 and 8 compared to 6). It is also noticeable that rhlB exhibits better performance that dam expression in these cases and can potentially improve the system in the context of HR.

### Example 8: Error-Rate Estimations for TF1 RT

In order to evaluate the in vivo error profile of TF1 reverse transcriptase, bMS_453 cells were double transformed with VN1270+VN1269 (system 1) or VN1237+VN1228 (system 2). VN1237 plasmid were previously described herein as VN1238 and VN1228 has also been previously described herein. VN1270 is a derivative of VN1237 B2H single plasmid by replacing the original antibiotic resistance gene (intended for chloramphenicol selection) by the Bla gene (for ampicillin selection). VN1269 is a modified version of the plasmid described by Schubert et al. (Schubert et al. bioRxiv 2020.03.05.975441; doi: https://doi.org/10.1101/2020.03.05.975441) which encodes a chlorampenicol resistance gene and is intended for retron reverse-transcriptase based edition of the same locus target by VN1228 (i.e., ShBle Stop that invalidate zeocin resistance).

The transformed cells were culture in LB containing ampicillin (75 μg/ml) and chloramphenicol (25 μg/ml) (31° C., 190 rpm, overnight). Then, fresh dilutions were made from saturated cultures in 50 ml tubes (O.D.₆₀₀ₙₘ=0.01, 10 ml) and kept at 31° C. for 1 hour and 30 minutes (O.D.₆₀₀ₙₘ<0.3) when system 1 was induced by arabinose (50 mM) and IPTG (20 nM) while system 2 was induced by aTc (200 ng/ml) and IPTG (20 nM). Next, the cultures were incubated in a thermomixer (Eppendorf) at 42° C., 900 rpm, for 14 minutes and put back at 31° C., 190 rpm for about 6h and 30 minutes. Finally, 10⁸ cells of the obtained culture (O.D.₆₀₀ₙₘ˜3.0) were inoculated into 10 ml of LB containing zeocin (20 μg/ml) and IPTG (20 μM).

The plasmids were extracted from zeocin resistant cells and used as template for PCR reactions (˜350 ng for 100 μl reactions) designed for the amplification of the targeted region in the B2H plasmids (i.e. ShBle Stop in VN1237 or VN1270) using Q5 polymerase. The PCR products were agarose gel purified and used (0,062 μmol) in a 3-way golden gate reaction (10 μl; NEB, Golden Gate Assembly Kit BsaI-HF® v2, E1601S) with 5′ adaptor fragment (0,025 μmol) and 3′ adaptor fragment (0,025 pmol). 5′ and 3′ fragments contained demultiplexing and UMI (unique molecular identifier) sequences and required regions for Illumina NGS. Ligated products, were column purified (GeneJET PCR Purification, Thermo, K0701) and PCR amplified using 5′ and 3′ primers, the product of the expected size was gel purified and sequenced (2×150 paired-end reads, Illumina NOVASEQ 6000 platform, NOVOGEN, UK). To decrease sequencing errors, the cDNA targeted region was fully covered by both paired-end reads in order to reconstruct high quality assemblies for bioinformatics analysis. This strategy allows the efficient deep sequencing of single molecules in order to improve statistics reliability and to suppress sequencing errors. In the one hand, under the described conditions, system 1 (retron based edition) shows 27.35% of mutated sequences (in other words 72.65% of the sequences corresponded to the expected product—faithful to the presented reverse transcription template). In the other hand, system 2 (TF1 RT based using the described concepts) resulted in 99.81% mutated sequences. Focused analysis of the mutated sequences indicate higher insertion frequency for system 2 (7.65E-03 insertion per base) compared to system 1 (3.25E-05 insertion per base). The majority of these events correspond to “A” insertions in poly-A regions for system 2, which is compatible with previously described TF1 RT profile (Kirshenboim et al., Virology. 2007 Sep. 30; 366(2):263-76. doi: 10.1016/j.virol.2007.04.002. Epub 2007 May 23. PMID: 17524442). Similar frequencies of mutation by nucleotide misincorporation were observed for both systems (System 1: 7.34E-04 mutations per base; System 2: 6.37E-04 mutations per base).

>NGS Full amplicon sequence double tagged with
unique molecular identifiers (UMIs)
(SEQ ID NO: 71)

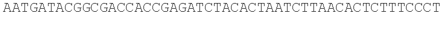

ACACGACGCTCTTCCGATCTCGCHHNHHNHATTCGGAAGCTTTCGTTGAC
TTACGTGATGTACGTCAGCCTGAAGTGAAAGAAGAGAAACCAGAGGCGGC
CGCAGCCAAGTTGACCAGTGCAGTTCCGGTGCTCACCGCGCGCGACGTCG
CCGGAGCGGTCGAGTTCTGGACCGACCGGCTCGGGTTCTCACCTDNDDND
DGCGAGATCGGAAGAGCACACGTCTGAACTCCAGTCACCTTGCTAGATCT

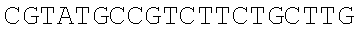

UMI (Unique Molecular Identifier): the corresponding region is indicated in bold and the subregion of variable size is underlined (three sequences are expected at this site: CGC, CT or A). [symbol] for the amplification of the full DNA fragment for NGS sequencing (Illumina platform) is indicated.

For each UMI, the constant size region (HHNHHNH or DNDDNDD) corresponds to 3888 sequences that can be found fused to 3 different variable regions for a total of 11664 possible UMIs. By combining the UMIs at both sides a theoretical diversity of 136 048 896 is achieved.

>5’ fragment
(SEQ ID NO: 72)
AATGATACGGCGACCACCGAGATCTACACTAATCTTAACACTCTTTCCC
TACACGACGCTCTTCCGATCTCGCHHNHHNHATTCTGAGACCTTTCCC
>3’fragment
(SEQ ID NO: 73)
GGGAAAGGTCTCAACCTDNDDNDDGCGAGATCGGAAGAGCACACGTCTG
AACTCCAGTCACCTTGCTAGATCTCGTATGCCGTCTTCTGCTTG
>Internal fragment
(SEQ ID NO: 74)
GGGAAAGGTCTCAATTCGGAAGCTTTCGTTGACTTACGTGATGTACGTC
AGCCTGAAGTGAAAGAAGAGAAACCAGAGGCGGCCGCAGCCAAGTTGAC
CAGTGCAGTTCCGGTGCTCACCGCGCGCGACGTCGCCGGAGCGGTCGAG
TTCTGGACCGACCGGCTCGGGTTCTCACCTTGAGACCTTTCCC
>OR2004: Internal fragment forward primer
(SEQ ID NO: 75)
GGGAAAGGTCTCAATTCGGAAGCTTTCGTTGACTTACG
>OR2005: Internal fragment reverse primer
(SEQ ID NO: 76)
GGGAAAGGTCTCAAGGTGAGAACCCGAGCCGG

1. A method for generating diversity in a gene L, comprising:
providing a bacterial cell comprising a molecular complex formed by the association of:
a scaffold protein (SP),
a template RNA (tpRNA) comprising from 5′ to 3′: the gene L, an RTtag sequence operably linked to the gene L and a scaffold protein binding module 1 (SPBM1) sequence capable of binding to the SP at a first specific binding site (SPS1).
a primer RNA (prRNA) comprising: an RTprimer sequence positioned in 3′ end of the prRNA and capable of complementary pairing to the RTtag sequence, a scaffold protein binding module 2 (SPBM2) sequence capable of binding to the SP at a second specific binding site (SPS2) and a reverse transcriptase binding module (RBM) sequence,
a fusion protein (RBD-RT) comprising a reverse transcriptase (RT) and an RBM binding domain (RBD) capable of binding to the RBM of the prRNA.
placing the bacterial cell in conditions that allow the reverse transcription of the gene L, thereby generating altered copies of said gene L of the tpRNA.

2-38. (canceled)


SEQUENCE LISTING (76 sequences)

SEQ ID NO: 1 - protein, 505 aa, artificial sequence
  Tf1 RT
ISSSKHTLSQMNKVSNIVKEPELPDIYKEFKDITADTNTEKLPKPIKGLEFEVELTQENYRLPIRNYPLPPGKMQAMNDEINQGLKSGIIRESKAINACPVMFVPKKEGTLRMVVDYKPLNKYVKPNIYPLPLIEQLLAKIQGSTIFTKLDLKSAYHLIRVRKGDEHKLAFRCPRGVFEYLVMPYGISTAPAHFQYFINTILGEAKESHVVCYMDDILIHSKSESEHVKHVKDVLQKLKNANLIINQAKCEFHQSQVKFIGYHISEKGFTPCQENIDKVLQWKQPKNRKELRQFLGSVNYLRKFIPKTSQLTHPLNKLLKKDVRWKWTPTQTQAIENIKQCLVSPPVLRHFDFSKKILLETDASDVAVGAVLSQKHDDDKYYPVGYYSAKMSKAQLNYSVSDKEMLAIIKSLKHWRHYLESTIEPFKILTDHRNLIGRITNESEPENKRLARWQLFLQDFNFEINYRPGSANHIADALSRIVDETEPIPKDSEDNSINFVNQISI

SEQ ID NO: 2 - protein, 561 aa, artificial sequence
  HIV-1 RT p66
MPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKQKKSVTVLDVGDAYFSVPLDKDFRKYTAFTIPSINNETPGIRYQYNVLPQGWKGSPAIFQCSMTKILEPFRKQNPDIVIYQYMDDLYVGSDLEIGQHRTKIEELRQHLLRWGFTTPDKKHQKEPPFLWMGYELHPDKWTVQPIVLPEKDSWTVNDIQKLVGKLNWASQIYAGIKVRQLCKLLRGTKALTEVVPLTEEAELELAENREILKEPVHGVYYDPSKDLIAEIQKQGQGQWTYQIYQEPFKNLKTGKYARMKGAHTNDVKQLTEAVQKIATESIVIWGKTPKFKLPIQKETWEAWWTEYWQATWIPEWEFVNTPPLVKLWYQLEKEPIIGAETFYVDGAANRETKLGKAGYVTDRGRQKVVPLTDTTNQKTELQAIHLALQDSGLEVNIVTDSQYALGIIQAQPDKSESELVSQIIEQLIKKEKVYLAWVPAHKGIGGNEQVDGLVSAGIRKVL

SEQ ID NO: 3 - protein, 627 aa, artificial sequence
  MMLV RT
MGQPLQVLTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEIFK

SEQ ID NO: 4 - DNA, 77 bp, artificial sequence
  tRNA
cggtgattggcgcagcctggtagcgcacttcgttcgggacgaaggggtcggaggttcgaatcctctatcaccgacca

SEQ ID NO: 5 - protein, 22 aa, artificial sequence
  Lambda phage N protein (1-22) domain
MDAQTRRRERRAEKQAQWKAAN

SEQ ID NO: 6 - DNA, 66 bp, artificial sequence
  CDS Lambda phage N protein domain
atggacgcacaaacacgccgccgcgaacgtcgcgcagagaaacaggctcaatggaaagcagcaaat

SEQ ID NO: 7 - RNA, 15 nt, artificial sequence
  Lambda BoxB from nutL
gcccugaagaagggc

SEQ ID NO: 8 - protein, 130 aa, artificial sequence
  MS2 phage coat protein/ Genbank accession FJ799575.1
MASNFTQFVLVDNGGTGDVTVAPSNFANGVAEWISSNSRSQAYKVTCSVRQSSAQNRKYTIKVEVPKVATQTVGGVELPVAAWRSYLNMELTIPIFATNPDCELIVKAMQGLLKDGNPIPSAIAANSGIY

SEQ ID NO: 9 - DNA, 23 bp, artificial sequence
  WT MS2 binding motif
aaacatgaggattacccatgtcg

SEQ ID NO: 10 - DNA, 23 bp, artificial sequence
  high affinity MS2 binding motif
aaacatgaggatcacccatgtcg

SEQ ID NO: 11 - protein, 128 aa, artificial sequence
  PP7 phage coat protein
MSKTIVLSVGEATRTLTEIQSTADRQIFEEKVGPLVGRLRLTASLRQNGAKTAYRVNLKLDQADVVDCSTSVCGELPKVRYTQVWSHDVTIVANSTEASRKSLYDLTKSLVATSQVEDLVVNLVPLGR

SEQ ID NO: 12 - RNA, 25 nt, artificial sequence
  PP7 binding motif
ggcacagaagauauggcuucgugcc

SEQ ID NO: 13 - DNA, 20 bp, artificial sequence
  RTprimer
tctagtgctttactctcatt

SEQ ID NO: 14 - DNA, 20 bp, artificial sequence
  RTtag
aatgagagtaaagcactaga

SEQ ID NO: 15 - protein, 102 aa, artificial sequence
  Hfq protein
MAKGQSLQDPFLNALRRERVPVSIYLVNGIKLQGQIESFDQFVILLKNTVSQMVYKHAISTVVPSRPVSHHSNNAGGGTSSNYHHGSSAQNTSAQQDSEETE

SEQ ID NO: 16 - protein, 65 aa, artificial sequence
  Hfq (delta)C-term
MAKGQSLQDPFLNALRRERVPVSIYLVNGIKLQGQIESFDQFVILLKNTVSQMVYKHAISTVVPS

SEQ ID NO: 17 - DNA, 49 bp, artificial sequence
  SPBM1
tattggtgtaaaatcacccgccagcagattatacctgctggtttttttt

SEQ ID NO: 18 - DNA, 21 bp, artificial sequence
  SPBM2
aacaacaacaacaacaacaac

SEQ ID NO: 19 - protein, 261 aa, artificial sequence
  Lambda Bet
MSTALATLAGKLAERVGMDSVDPQELITTLRQTAFKGDASDAQFIALLIVANQYGLNPWTKEIYAFPDKQNGIVPVVGVDGWSRIINENQQFDGMDFEQDNESCTCRIYRKDRNHPICVTEWMDECRREPFKTREGREITGPWQSHPKRMLRHKAMIQCARLAFGFAGIYDKDEAERIVENTAYTAERQPERDITPVNDETMQEINTLLIALDKTWDDDLLPLCSQIFRRDIRASSELTQAEAVKALGFLKQKAAEQKVAA

SEQ ID NO: 20 - DNA, 24 bp, artificial sequence
  RBS7
tagaccagctcgctaggtcatata

SEQ ID NO: 21 - DNA, 36 bp, artificial sequence
  pLTetO promoter
ttgacatccctatcagtgatagagatactgctagca

SEQ ID NO: 22 - protein, 237 aa, artificial sequence
  DBD from cI
MSTKKKPLTQEQLEDARRLKAIYEKKKNELGLSQPSVADKMGMGQSGVGALFNGINALNAYNAALLAKILKVSVEEFSPSIAREIYEMYEAVSMQPSLRSEYEYPVFSHVQAGMFSPELRTFTKGDAERWVSTTKKASDSAFWLEVEGNSMTAPTGSKPSFPDGMLILVDPEQAVEPGDFCIARLGGDEFTFKKLIRDSGQVFLQPLNPQYPMIPCNESCSVVGKVIASQWPEETFG

SEQ ID NO: 23 - protein, 248 aa, artificial sequence
  subunit alpha from RNA polymerase
MQGSVTEFLKPRLVDIEQVSSTHAKVTLEPLERGFGHTLGNALRRILLSSMPGCAVTEVEIDGVLHEYSTKEGVQEDILEILLNLKGLAVRVQGKDEVILTLNKSGIGPVTAADITHDGDVEIVKPQHVICHLTDENASISMRIKVQRGRGYVPASTRIHSEEDERPIGRLLVDACYSPVERIAYNVEAARVEQRTDLDKLVIEMETNGTIDPEEAIRRAATILAEQLEAFVDLRDVRQPEVKEEKPE

SEQ ID NO: 24 - DNA, 71 bp, artificial sequence
  epB2H
caacaccgccagagatacattaggcaccggcggcttgacactttatgcttccggctcggatactgtgtgga

SEQ ID NO: 25 - protein, 500 aa, artificial sequence
  DNA invertase BxB1
MRALVVIRLSRVTDATTSPERQLESCQQLCAQRGWDVVGVAEDLDVSGAVDPFDRKRRPNLARWLAFEEQPFDVIVAYRVDRLTRSIRHLQQLVHWAEDHKKLVVSATEAHFDTTTPFAAVVIALMGTVAQMELEAIKERNRSAAHFNIRAGKYRGSLPPWGYLPTRVDGEWRLVPDPVQRERILEVYHRVVDNHEPLHLVAHDLNRRGVLSPKDYFAQLQGREPQGREWSATALKRSMISGAMLGYATLNGKTVRDDDGAPLVRAEPILTREQLEALRAELVKTSRAKPAVSTPSLLLRVLFCAVCGEPAYKFAGGGRKHPRYRCRSMGFPKHCGNGTVAMAEWDAFCEEQVLDLLGDAERLEKVWVAGSDSAVELAEVNAELVDLTSLIGSPAYRAGSPQREALDARIAALAARQEELEGLEARPSGWEWRETGQRFGDWWREQDTAAKNTWLRSMNVRLTFDVRGGLTRTIDFGDLQEYEQHLRLGSVVERLHTGMS

SEQ ID NO: 26 - DNA, 50 bp, artificial sequence
  attB (DNA invertase site)
tcggccggcttgtcgacgacggcggtctccgtcgtcaggatcatccgggc

SEQ ID NO: 27 - DNA, 58 bp, artificial sequence
  attP (DNA invertase site)
gtcgtggtttgtctggtcaaccaccgcggtctcagtggtgtacggtacaaaccccgac

SEQ ID NO: 28 - protein, 207 aa, artificial sequence
  tetR repressor
MSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGS

SEQ ID NO: 29 - protein, 102 aa, artificial sequence
  PDZ domain
LGEEDIPREPRRIVIHRGSTGLGFNIVGGEDGEGIFISFILAGGPADLSGELRKGDQILSVNGVDLRNASHEQAAIALKNAGQTVTIIAQYKPEEYSRFEAK

SEQ ID NO: 30 - protein, 9 aa, artificial sequence
  CRIPT peptide
TKNYKQTSV

SEQ ID NO: 31 - protein, 5 aa, artificial sequence
  G4S
GGGGS

SEQ ID NO: 32 - protein, 219 aa, artificial sequence
  Chloramphenicol resistance protein
MEKKITGYTTVDISQWHRKEHFEAFQSVAQCTYNQTVQLDITAFLKTVKKNKHKFYPAFIHILARLMNAHPEFRMAMKDGELVIWDSVHPCYTVFHEQTETFSSLWSEYHDDFRQFLHIYSQDVACYGENLAYFPKGFIENMFFVSANPWVSFTSFDLNVANMDNFFAPVFTMGKYYTQGDKVLMPLAIQVHHAVCDGFHVGRMLNELQQYCDEWQGGA

SEQ ID NO: 33 - protein, 239 aa, artificial sequence
  enhanced Green Fluorescent Protein (eGFP)
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 34 - protein, 272 aa, artificial sequence
  kanamycin resistance protein
MISHIQRETSCSRPRLNSNMDADLYGYKWARDNVGQSGATIYRLYGKPDAPELFLKHGKGSVANDVTDEMVRLNWLTEFMPLPTIKHFIRTPDDAWLLTTAIPGKTAFQVLEEYPDSGENIVDALAVFLRRLHSIPVCNCPFNSDRVFRLAQAQSRMNNGLVDASDFDDERNGWPVEQVWKEMHKLLPFSPDSVVTHGDFSLDNLIFDEGKLIGCIDVGRVGIADRYQDLAILWNCLGEFSPSLQKRLFQKYGIDNPDMNKLQFHLMLDEFF

SEQ ID NO: 35 - protein, 263 aa, artificial sequence
  streptomycin, spectinomycin resistance protein
MREAVIAEVSTQLSEVVGVIERHLEPTLLAVHLYGSAVDGGLKPHSDIDLLVTVTVRLDETTRRALINDLLETSASPGESEILRAVEVTIVVHDDIIPWRYPAKRELQFGEWQRNDILAGIFEPATIDIDLAILLTKAREHSVALVGPAAEELFDPVPEQDLFEALNETLTLWNSPPDWAGDERNVVLTLSRIWYSAVTGKIAPKDVAADWAMERLPAQYQPVILEARQAYLGQEEDRLASRADQLEEFVHYVKGEITKVVGK

SEQ ID NO: 36 - protein, 122 aa, artificial sequence
  Zeocin resistance protein
KLTSAVPVLTARDVAGAVEFWTDRLGFSRDFVEDDFAGVVRDDVTLFISAVQDQVVPDNTLAWVWVRGLDELYAEWSEVVSTNFRDASGPAMTEIGEQPWGREFALRDPAGNCVHFVAEEQD

SEQ ID NO: 37 - DNA, 3485 bp, artificial sequence
  plasmid VN591
tatttctagatttcagtgcaatttatctcttcaaatgtagcacctgaagtcagccccatacgatataagttgtaattctcatgtttgacagcttatcatcgataagctaattctcactcattaggcaccccaggctttacactttatgcttccggctcgtataatgtgtggaattgtgagcggataacaatttcacacaggaaacagcatatgagcgcaatattaaacggtagataaccaagaaaacatctcatattcaacgggaaacgtcttgctctaggccgcgattaaattccaacatggatgctgatttatatgggtataaatgggcccgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatgaggtaacctattattgctcgagtcacactggctcaccttcgggtgggcctttctgcgtttatatactagagagagaatataaaaagccagattattaatccggcttttttattatttagttacgctagggataacagggtaatatagggtaccatgattgagctcctattaaagctttaatgcggatccttaccttacagatctgcatcgcaggatgctgctggctaccctgtggaacacctacatctgtattaacgaagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgaatgctgccaacttactgattagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaa

SEQ ID NO: 38 - DNA, 7592 bp, artificial sequence
  plasmid VN575
gcaataaaaaatcaaatcggatttcactatataatctcactttatctaagatgaatccgatggaagcatcctgttttctctcaatttttttatctaaaacccagcgttcgatgcttctttgagcgaacgatcaaaaataagtgccttctctagattttcagcaggacgcactgacctccctatcagtgatagagattgacatccctatcagtgatagagatactgagcacatatataagcttcctacttacttacttaaacactacggttaattatatggcaactctgaacatcgaagatgagcatcgcttgcacgaaaccagcaaagagcctgatgtttctctgggctcgacatggttgagtgacttcccgcaggcctgggctgaaacgggaggaatgggtctggcggtacgccaagcaccgctgattattccgctgaaagcaaccagcacccctgtgtcgatcaaacagtacccgatgagccagaaagcccgcctgggtatcaaaccccacattcagcgcttgctggaccaaggcattttggtgccgtgtcagtccccgtggaatactcctttactgccggtgaaaaaacccggcaccaacgattatcgtccggtccaggatttgcgcgaggttaacaaacgtgttgaggacattcacccgaccgttcccaatccatacaacctcctgtcaggcctgcctccatcacatcaatggtataccgtgttagacctgaaagatgcattcttttgcctgcgtttacatccgacgagccagcctcttttcgcttttgagtggcgggatccggagatgggcatttccggacagctgacatggactcgcctgccgcagggcttcaagaatagcccaacgctgtttgacgaagcgttgcatcgtgatctggccgatttccgcatccagcatccggacttaattctcttgcagtacgtggacgatttactgttggccgcaacgtctgaactggattgccaacaggggacacgcgcgctgctgcagacgctcggcaacctgggttatcgcgcatcggcgaaaaaagcccagatctgccagaaacaagtcaaatacctcggctacttgttaaaagaaggccaacgctggctgactgaagcgcggaaagaaaccgtgatggggcaaccaaccccgaaaacaccacgtcagctgcgtcgctttctgggtaccgcaggcttttgtcgcctgtttatccccgggtttgccgaaatggcagctccgttgtatccgctgaccaaaaccggtaccctgttcaactggggcccggatcagcagaaagcatatcaggaaatcaaacaggcgcttctgaccgctcccgccctgggtctgccggaccttacaaaaccgtttgaactgtttgtagacgagaaacaaggttatgcgaaaggggtacttacgcagaagctgggcccttggcgtcgtccagtggcttatctgtccaagaaactggatccggtagcagcgggctggcctccttgtctgcgcatggtcgccgcgattgccgtgctgactaaggatgcgggcaaactcacgatgggccaaccactggttattggtgctccacatgcggtcgaagcgttagtgaaacagccgcctgatcgttggctgagtaaggcgcgcatgacgcactatcaagccctgcttctggataccgatcgcgtgcagttcggaccggttgttgccctgaatccggctaccctgttacccctgccggaagaagggttacagcacaattgcttggacattctggcggaagcgcatggtactcgtcccgacctgacggaccaaccgttaccggatgccgatcacacctggtatacgaatggcagttcgctccttcaagagggtcaacgtaaggcgggtgctgcggtcaccacggaaaccgaagtgatttgggctaaagcgctcccagcgggaacgtcagcgcaacgtgccgaactgattgctcttacccaggcccttaaaatggcagagggtaagaaacttaatgtctacaccgatagccgctatgcgtttgctactgcgcatatccacggtgaaatttaccgtcgtcgcggtctgctgacgagtgaagggaaagaaatcaagaacaaagatgaaattctggcgctcttaaaggcgctcttcttaccaaagcgtttgtccatcattcactgtccgggccatcagaaaggtcattctgcagaggcacggggcaatcggatggccgatcaggccgcacgcaaagccgcaatcaccgaaacaccggataccagcactctcctgcgtgatcgcaacaagaacaacgaccgccgcaaagccaaagaaaatgagtaatgaagatctatacaggtcaaaatactacggttacatataatgagtactgcactcgcaacgctggctgggaagctggctgaacgtgtcggcatggattctgtcgacccacaggaactgatcaccactcttcgccagacggcatttaaaggtgatgccagcgatgcgcagttcatcgcattactgatcgttgccaaccagtacggccttaatccgtggacgaaagaaatttacgcctttcctgataagcagaatggcatcgttccggtggtgggcgttgatggctggtcccgcatcatcaatgaaaaccagcagtttgatggcatggactttgagcaggacaatgaatcctgtacatgccggatttaccgcaaggaccgtaatcatccgatctgcgttaccgaatggatggatgaatgccgccgcgaaccattcaaaactcgcgaaggcagagaaatcacggggccgtggcagtcgcatcccaaacggatgttacgtcataaagccatgattcagtgtgcccgtctggccttcggatttgctggtatctatgacaaggatgaagccgagcgcattgtcgaaaatactgcatacactgcagaacgtcagccggaacgcgacatcactccggttaacgatgaaaccatgcaggagattaacactctgctgatcgccctggataaaacatgggatgacgacttattgccgctctgttcccagatatttcgccgcgacattcgtgcatcgtcagaactgacacaggccgaagcagtaaaagctcttggattcctgaaacagaaagccgcagagcagaaggtggcagcatgaggtaatgaacgcgttcacactggctcaccttcgggtgggcctttctgcgtttatatactagagagagaatataaaaagccagattattaatccggcttttttattatttgcggccgccgatcccgcgaaattaatacgactcactataggcctagagcaagacgtttcccgttgaatatgagatgttttcttgggttaattgaggcctgagtataaggtgacttatacttgtaatctatctaaacggggaacctctctagtagacaatcccgtgctaaattgtaggactgccctttaataaatacttctatatttaaagaggtatttatgaaaagcggaatttatcagattaaaaatactttttaattaacaatgtagtggtgctggataaccgtacgaaaatttcgtctggattagttacttatcgtgtaaaatctgataaatggaattggttctacataaatgcctaacgactatccctttggggagtagggtcaagtgactcgaaacgatagacaacttgctttaacaagttggagatatagtctgctctgcatggtgacatgcagctggatataattccggggtaagattaacgaccttatctgaacataatgctaccgtttaatattgcgctcatatgctgtttcctgtgtgaaatactagtgcgcattgccagagtgaaaatgcctggcgcgcccctagcataaccccgcggggcctcttcgggggtctcgcggggttttttgctgaaagatcaaataaaacgaaaggctcagtcgaaagactgggcctttcgttttatctaaagatcagttaaccccttggggcctctaaacgggtcttgaggggttttttgctgaaagctcgagagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtcgagctcttaggacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctggacatcattaattcctaattttatgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgt

SEQ ID NO: 39 - DNA, 7494 bp, artificial sequence
  plasmid VN669
gcaataaaaaatcaaatcggatttcactatataatctcactttatctaagatgaatccgatggaagcatcctgttttctctcaatttttttatctaaaacccagcgttcgatgcttctttgagcgaacgatcaaaaataagtgccttctctagattttcagcaggacgcactgacctccctatcagtgatagagattgacatccctatcagtgatagagatactgagcacatatataagcttttttactaacgagcaaaatactaaggaggttactaatggacgcacaaacacgccgccgcgaacgtcgcgcagagaaacaggctcaatggaaagcagcaaatggaggtggcgggagtggtggcggaggtagcatggcaactctgaacatcgaagatgagcatcgcttgcacgaaaccagcaaagagcctgatgtttctctgggctcgacatggttgagtgacttcccgcaggcctgggctgaaacgggaggaatgggtctggcggtacgccaagcaccgctgattattccgctgaaagcaaccagcacccctgtgtcgatcaaacagtacccgatgagccagaaagcccgcctgggtatcaaaccccacattcagcgcttgctggaccaaggcattttggtgccgtgtcagtccccgtggaatactcctttactgccggtgaaaaaacccggcaccaacgattatcgtccggtccaggatttgcgcgaggttaacaaacgtgttgaggacattcacccgaccgttcccaatccatacaacctcctgtcaggcctgcctccatcacatcaatggtataccgtgttagacctgaaagatgcattcttttgcctgcgtttacatccgacgagccagcctcttttcgcttttgagtggcgggatccggagatgggcatttccggacagctgacatggactcgcctgccgcagggcttcaagaatagcccaacgctgtttgacgaagcgttgcatcgtgatctggccgatttccgcatccagcatccggacttaattctcttgcagtacgtggacgatttactgttggccgcaacgtctgaactggattgccaacaggggacacgcgcgctgctgcagacgctcggcaacctgggttatcgcgcatcggcgaaaaaagcccagatctgccagaaacaagtcaaatacctcggctacttgttaaaagaaggccaacgctggctgactgaagcgcggaaagaaaccgtgatggggcaaccaaccccgaaaacaccacgtcagctgcgtcgctttctgggtaccgcaggcttttgtcgcctgtttatccccgggtttgccgaaatggcagctccgttgtatccgctgaccaaaaccggtaccctgttcaactggggcccggatcagcagaaagcatatcaggaaatcaaacaggcgcttctgaccgctcccgccctgggtctgccggaccttacaaaaccgtttgaactgtttgtagacgagaaacaaggttatgcgaaaggggtacttacgcagaagctgggcccttggcgtcgtccagtggcttatctgtccaagaaactggatccggtagcagcgggctggcctccttgtctgcgcatggtcgccgcgattgccgtgctgactaaggatgcgggcaaactcacgatgggccaaccactggttattggtgctccacatgcggtcgaagcgttagtgaaacagccgcctgatcgttggctgagtaaggcgcgcatgacgcactatcaagccctgcttctggataccgatcgcgtgcagttcggaccggttgttgccctgaatccggctaccctgttacccctgccggaagaagggttacagcacaattgcttggacattctggcggaagcgcatggtactcgtcccgacctgacggaccaaccgttaccggatgccgatcacacctggtatacgaatggcagttcgctccttcaagagggtcaacgtaaggcgggtgctgcggtcaccacggaaaccgaagtgatttgggctaaagcgctcccagcgggaacgtcagcgcaacgtgccgaactgattgctcttacccaggcccttaaaatggcagagggtaagaaacttaatgtctacaccgatagccgctatgcgtttgctactgcgcatatccacggtgaaatttaccgtcgtcgcggtctgctgacgagtgaagggaaagaaatcaagaacaaagatgaaattctggcgctcttaaaggcgctcttcttaccaaagcgtttgtccatcattcactgtccgggccatcagaaaggtcattctgcagaggcacggggcaatcggatggccgatcaggccgcacgcaaagccgcaatcaccgaaacaccggataccagcactctcctgcgtgatcgcaacaagaacaacgaccgccgcaaagccaaagaaaatgagtaatgaagatctatacaggtcaaaatactacggttacatataatgagtactgcactcgcaacgctggctgggaagctggctgaacgtgtcggcatggattctgtcgacccacaggaactgatcaccactcttcgccagacggcatttaaaggtgatgccagcgatgcgcagttcatcgcattactgatcgttgccaaccagtacggccttaatccgtggacgaaagaaatttacgcctttcctgataagcagaatggcatcgttccggtggtgggcgttgatggctggtcccgcatcatcaatgaaaaccagcagtttgatggcatggactttgagcaggacaatgaatcctgtacatgccggatttaccgcaaggaccgtaatcatccgatctgcgttaccgaatggatggatgaatgccgccgcgaaccattcaaaactcgcgaaggcagagaaatcacggggccgtggcagtcgcatcccaaacggatgttacgtcataaagccatgattcagtgtgcccgtctggccttcggatttgctggtatctatgacaaggatgaagccgagcgcattgtcgaaaatactgcatacactgcagaacgtcagccggaacgcgacatcactccggttaacgatgaaaccatgcaggagattaacactctgctgatcgccctggataaaacatgggatgacgacttattgccgctctgttcccagatatttcgccgcgacattcgtgcatcgtcagaactgacacaggccgaagcagtaaaagctcttggattcctgaaacagaaagccgcagagcagaaggtggcagcatgaggtaatgaacgcgtttcagccaaaaaacttaagaccgccggtcttgtccactaccttgcagtaatgcggtggacaggatcggcggttttcttttctcttctcaagcggccgccgatcccgcgaaatttgacatccctatcagtgatagatataatgctagcaatcgcggcctagagcaagacgtttcccgttgaatatgagatgttttcttgggtctaccgtttaatattgcgctcatatgctgtttcctgtgtgaaatactagtaatgtgtcatgcatgtgtcctattggtgtaaaatcacccgccagcagattatacctgctggttttttttggcgcgccctcggtaccaaattccagaaaagaggcctcccgaaaggggggccttttttcgttttggtccttcgtccatggaggcattttgctattaagggattgacgtccctatcagtgatagataagatgcgccccgcattgggccctgaagaagggcccgggccctgaagaagggcccaacaacaacaacaacaacaacggacacatgcatgacacattcggtgattggcgcagcctggtagcgcacttcgttcgggacgaaggggtcggaggttcgaatcctctatcaccgaccaaattcgaaaagcctgctcaacgagcaggcttttttgcatctcgagagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtcgagctcttaggacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctggacatcattaattcctaattttatgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgt

SEQ ID NO: 40 - DNA, 6135 bp, artificial sequence
  plasmid VN520
cgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggtgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccagagcctgataaaaacggttagcgcttcgttaatacagatgtaggtgttccacagggtagccagcagcatcctgcgatgcagatctgtaaggtaaggatccttatcagttatcttcccaattaatgtggaatcttgtgaccctgggattagatgccaaaatattcctttgaagcttagaaaagtctggttttactggtggattttcccttaattctgtctcagtatattcattatttacataatagccaactctaataaattcttgtcctcgataggtacaagtaattagcacaacagttacgcctactgcatctgcatctggaatgagtcctggattaggtgcatcagcctgaaatacaaacatatgccttcctgcgggaacaggacccactaaaacagagtctaaaacttgatcgtattcttcactttctgcagagcccacatagataattttccattccaagtcttcagacaggtcctcgatgcactcgaaggtgatctcgaactggaacgggttgtagaaaggagaagggttatccagcaccactacattgttcacctgaacctttgctgcggccgcgccaaacgtctcttcaggccactgactagcgataactttccccacaacggaacaactctcattgcatgggatcattgggtactgtgggtttagtggttgtaaaaacacctgaccgctatccctgatcagtttcttgaaggtaaactcatcacccccaagtctggctatgcagaaatcacctggctcaacagcctgctcagggtcaacgagaattaacattccgtcaggaaagcttggcttggagcctgttggtgcggtcatggaattaccttcaacctcaagccagaatgcagaatcactggcttttttggttgtgcttacccatctctccgcatcacctttggtaaaggttctaagctcaggtgagaacatccctgcctgaacatgagaaaaaacagggtactcatactcacttctaagtgacggctgcatactaaccgcttcatacatctcgtagatttctctggcgattgaagggctaaattcttcaacgctaactttgagaatttttgcaagcaatgcggcgttataagcatttaatgcattgatgccattaaataaagcaccaacgcctgactgccccatccccatcttgtctgcgacagattcctgggataagccaagttcatttttctttttttcataaattgctttaaggcgacgtgcgtcctcaagctgctcttgtgttaatggtttcttttttgtgctcatacgctgtttcctgtgtgaaattgttatccgctcacaattccacacattatacgagccggaagcataaagtgtaaagcctggggtgcctaatgagtgagaattagcttatcgatgataagctgtcaaacatgagaattacaacttatatcgtatggggctgacttcaggtgctacatttgaagagataaattgcactgaaatctagaatgtctccagaccgctagtagacgagtccatgtgctggcgttcaaatttcgcagcagcggtttctttaccagactcgagagcgaggctcttcaagctcgcaaaaaaccccgcccctgacagggcggggttttttcgcgttcgatcccgcgaaatttaattaaccaacaccgccagagatacattaggcaccccaggctttacactttatgcttccggctcgtatgttgtgtggaattgaaagaggaacgtacgatggtcagtaaaggcgaagagttgtttacgggtgtagtaccgattttagtggaactcgatggagatgtgaatgggcataaattttcggtttcaggtgaaggtgaaggagatgccacctatggcaaactgacgctgaagttcatctgtacgacaggcaaactgcctgttccgtggccaacactggttactacgctgacctatggcgttcaatgcttttcgcgttatccggaccacatgaaacagcatgacttctttaaatccgcgatgcctgaaggttacgtacaagaacgcaccatcttcttcaaagatgacgggaactacaaaacccgtgcagaggtcaaatttgagggtgacacccttgtgaatcggattgagctgaaaggcattgactttaaagaagatggcaacattctgggtcataaactggagtacaactacaacagccataacgtgtatatcatggcggacaaacagaagaatgggatcaaagtgaatttcaagattcgccacaacattgaagatggtagtgtgcagttagccgaccactatcagcagaataccccgattggcgatggcccagtcttgctgccggataatcattaccttagcacccaatctgcgttaagcaaagatcccaacgaaaagcgcgatcacatggtcctcctggaatttgtgactgctgcaggcatcactttgggtatggatgaactgtataagtaaacgcgttaaacaggaaacagaagtccaaatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcagatctaaataataaaaaagccggattaataatctggctttttatattctctctctagtatataaacgcagaaaggcccacccgaaggtgagccagtgtgacgaacccccgcctcagagtgggaaagtctagaaatattttatctgattaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccaggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgatgtccggcggtgcttttgccgttacgcaccaccccgtcagtagctgaacaggagggacagctgatagaaacagaagccactggagcacctcaaaaacaccatcatacactaatcagtaagttggcagcattcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattc

SEQ ID NO: 41 - DNA, 6189 bp, artificial sequence
  plasmid VN552
cgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggtgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccagagcctgataaaaacggttagcgcttcgttaatacagatgtaggtgttccacagggtagccagcagcatcctgcgatgcagatctgtaaggtaaggatccttatcagttatcttcccaattaatgtggaatcttgtgaccctgggattagatgccaaaatattcctttgaagcttagaaaagtctggttttactggtggattttcccttaattctgtctcagtatattcattatttacataatagccaactctaataaattcttgtcctcgataggtacaagtaattagcacaacagttacgcctactgcatctgcatctggaatgagtcctggattaggtgcatcagcctgaaatacaaacatatgccttcctgcgggaacaggacccactaaaacagagtctaaaacttgatcgtattcttcactttctgcagagcccacatagataattttccattccaagtcttcagacaggtcctcgatgcactcgaaggtgatctcgaactggaacgggttgtagaaaggagaagggttatccagcaccactacattgttcacctgaacctttgctgcggccgcgccaaacgtctcttcaggccactgactagcgataactttccccacaacggaacaactctcattgcatgggatcattgggtactgtgggtttagtggttgtaaaaacacctgaccgctatccctgatcagtttcttgaaggtaaactcatcacccccaagtctggctatgcagaaatcacctggctcaacagcctgctcagggtcaacgagaattaacattccgtcaggaaagcttggcttggagcctgttggtgcggtcatggaattaccttcaacctcaagccagaatgcagaatcactggcttttttggttgtgcttacccatctctccgcatcacctttggtaaaggttctaagctcaggtgagaacatccctgcctgaacatgagaaaaaacagggtactcatactcacttctaagtgacggctgcatactaaccgcttcatacatctcgtagatttctctggcgattgaagggctaaattcttcaacgctaactttgagaatttttgcaagcaatgcggcgttataagcatttaatgcattgatgccattaaataaagcaccaacgcctgactgccccatccccatcttgtctgcgacagacggctgggataagccaagttcatttttctttttttcataaattgctttaaggcgacgtgcgtcctcaagctgctcttgtgttaatggtttcttttttgtgctcatacgctgtttcctgtgtgaaattgttatccgctcacaattccacacattatacgagccggaagcataaagtgtaaagcctggggtgcctaatgagtgagaattagcttatcgatgataagctgtcaaacatgagaattacaacttatatcgtatggggctgacttcaggtgctacatttgaagagataaattgcactgaaatctagaatgtctccagaccgctagtagacgagtccatgtgctggcgttcaaatttcgcagcagcggtttctttaccagactcgagagcgaggctcttcaagctcgcaaaattgctcttcaagctcgcaaaaaaccccgcccctgacagggcggggttttttcgcgttcgatcccgcgaaatttaattaacctatcaccgccagaggtaaaatagtcaacacgcacggtgttagatatttatcccttgtggtgatagatttaacgtatcagcacaaaaaagaaaccgaattcattaaagaggagaaaacgtacgatggtcagtaaaggcgaagagttgtttacgggtgtagtaccgattttagtggaactcgatggagatgtgaatgggcataaattttcggtttcaggtgaaggtgaaggagatgccacctatggcaaactgacgctgaagttcatctgtacgacaggcaaactgcctgttccgtggccaacactggttactacgctgacctatggcgttcaatgcttttcgcgttatccggaccacatgaaacagcatgacttctttaaatccgcgatgcctgaaggttacgtacaagaacgcaccatcttcttcaaagatgacgggaactacaaaacccgtgcagaggtcaaatttgagggtgacacccttgtgaatcggattgagctgaaaggcattgactttaaagaagatggcaacattctgggtcataaactggagtacaactacaacagccataacgtgtatatcatggcggacaaacagaagaatgggatcaaagtgaatttcaagattcgccacaacattgaagatggtagtgtgcagttagccgaccactatcagcagaataccccgattggcgatggcccagtcttgctgccggataatcattaccttagcacccaatctgcgttaagcaaagatcccaacgaaaagcgcgatcacatggtcctcctggaatttgtgactgctgcaggcatcactttgggtatggatgaactgtataagtaaacgcgttaaacaggaaacagaagtccaaatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcagatctaaataataaaaaagccggattaataatctggctttttatattctctctctagtatataaacgcagaaaggcccacccgaaggtgagccagtgtgacgaacccccgcctcagagtgggaaagtctagaaatattttatctgattaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccaggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgatgtccggcggtgcttttgccgttacgcaccaccccgtcagtagctgaacaggagggacagctgatagaaacagaagccactggagcacctcaaaaacaccatcatacactaatcagtaagttggcagcattcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattc

SEQ ID NO: 42 - DNA, 6156 bp, artificial sequence
  plasmid VN550
cgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggtgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccagagcctgataaaaacggttagcgcttcgttaatacagatgtaggtgttccacagggtagccagcagcatcctgcgatgcagatctgtaaggtaaggatccttatcagttatcttcccaattaatgtggaatcttgtgaccctgggattagatgccaaaatattcctttgaagcttagaaaagtctggttttactggtggattttcccttaattctgtctcagtatattcattatttacataatagccaactctaataaattcttgtcctcgataggtacaagtaattagcacaacagttacgcctactgcatctgcatctggaatgagtcctggattaggtgcatcagcctgaaatacaaacatatgccttcctgcgggaacaggacccactaaaacagagtctaaaacttgatcgtattcttcactttctgcagagcccacatagataattttccattccaagtcttcagacaggtcctcgatgcactcgaaggtgatctcgaactggaacgggttgtagaaaggagaagggttatccagcaccactacattgttcacctgaacctttgctgcggccgcgccaaacgtctcttcaggccactgactagcgataactttccccacaacggaacaactctcattgcatgggatcattgggtactgtgggtttagtggttgtaaaaacacctgaccgctatccctgatcagtttcttgaaggtaaactcatcacccccaagtctggctatgcagaaatcacctggctcaacagcctgctcagggtcaacgagaattaacattccgtcaggaaagcttggcttggagcctgttggtgcggtcatggaattaccttcaacctcaagccagaatgcagaatcactggcttttttggttgtgcttacccatctctccgcatcacctttggtaaaggttctaagctcaggtgagaacatccctgcctgaacatgagaaaaaacagggtactcatactcacttctaagtgacggctgcatactaaccgcttcatacatctcgtagatttctctggcgattgaagggctaaattcttcaacgctaactttgagaatttttgcaagcaatgcggcgttataagcatttaatgcattgatgccattaaataaagcaccaacgcctgactgccccatccccatcttgtctgcgacagacggctgggataagccaagttcatttttctttttttcataaattgctttaaggcgacgtgcgtcctcaagctgctcttgtgttaatggtttcttttttgtgctcatacgctgtttcctgtgtgaaattgttatccgctcacaattccacacattatacgagccggaagcataaagtgtaaagcctggggtgcctaatgagtgagaattagcttatcgatgataagctgtcaaacatgagaattacaacttatatcgtatggggctgacttcaggtgctacatttgaagagataaattgcactgaaatctagaatgtctccagaccgctagtagacgagtccatgtgctggcgttcaaatttcgcagcagcggtttctttaccagactcgagagcgaggctcttcaagctcgcaaaattgctcttcaagctcgcaaaaaaccccgcccctgacagggcggggttttttcgcgttcgatcccgcgaaatttaattaaccaacaccgccagagatacattaggcaccccaggcttgacactttatgcttccggctcggatactgtgtggaattgaaagaggaacgtacgatggtcagtaaaggcgaagagttgtttacgggtgtagtaccgattttagtggaactcgatggagatgtgaatgggcataaattttcggtttcaggtgaaggtgaaggagatgccacctatggcaaactgacgctgaagttcatctgtacgacaggcaaactgcctgttccgtggccaacactggttactacgctgacctatggcgttcaatgcttttcgcgttatccggaccacatgaaacagcatgacttctttaaatccgcgatgcctgaaggttacgtacaagaacgcaccatcttcttcaaagatgacgggaactacaaaacccgtgcagaggtcaaatttgagggtgacacccttgtgaatcggattgagctgaaaggcattgactttaaagaagatggcaacattctgggtcataaactggagtacaactacaacagccataacgtgtatatcatggcggacaaacagaagaatgggatcaaagtgaatttcaagattcgccacaacattgaagatggtagtgtgcagttagccgaccactatcagcagaataccccgattggcgatggcccagtcttgctgccggataatcattaccttagcacccaatctgcgttaagcaaagatcccaacgaaaagcgcgatcacatggtcctcctggaatttgtgactgctgcaggcatcactttgggtatggatgaactgtataagtaaacgcgttaaacaggaaacagaagtccaaatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcagatctaaataataaaaaagccggattaataatctggctttttatattctctctctagtatataaacgcagaaaggcccacccgaaggtgagccagtgtgacgaacccccgcctcagagtgggaaagtctagaaatattttatctgattaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccaggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgatgtccggcggtgcttttgccgttacgcaccaccccgtcagtagctgaacaggagggacagctgatagaaacagaagccactggagcacctcaaaaacaccatcatacactaatcagtaagttggcagcattcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattc

SEQ ID NO: 43 - DNA, 5158 bp, artificial sequence
  plasmid VN515
gcaataaaaaatcaaatcggatttcactatataatctcactttatctaagatgaatccgatggaagcatcctgttttctctcaatttttttatctaaaacccagcgttcgatgcttctttgagcgaacgatcaaaaataagtgccttcccatcaaaaaaatattgacaacataaaaaactttgtgttatacttgtaacgctacatggagattaactcaatctagctagagaggctttacactttatgcttccggctcgtataatgtgtggaattgtgagcggataacaatttcacacaggaaacagctatgaccatgattacggattcactggaactctagaccaaagagaggacacaatgcagggttctgtgacagagtttctaaaaccgcgcctggttgatatcgagcaagtgagttcgacgcacgccaaggtgacccttgagcctttagagcgtggctttggccatactctgggtaacgcactgcgccgtattctgctctcatcgatgccgggttgcgcggtgaccgaggttgagattgatggtgtactacatgagtacagcaccaaagaaggcgttcaggaagatatcctggaaatcctgctcaacctgaaagggctggcggtgagagttcagggcaaagatgaagttattcttaccttgaataaatctggcattggccctgtgactgcagccgatatcacccacgacggtgatgtcgaaatcgtcaagccgcagcacgtgatctgccacctgaccgatgagaacgcgtctattagcatgcgtatcaaagttcagcgcggtcgtggttatgtgccggcttctacccgaattcattcggaagaagatgagcgcccaatcggccgtctgctggtcgacgcatgctacagccctgtggagcgtattgcctacaatgttgaagcagcgcgtgtagaacagcgtaccgacctggacaagctggtcatcgaaatggaaaccaacggcacaatcgatcctgaagaggcgattcgtcgtgcggcaaccattctggctgaacaactggaagctttcgttgacttacgtgatgtacgtcagcctgaagtgaaagaagagaaaccagaggcggccgcaggcgcgatgggcaccatcatgccgaaagatatccagctggcgcgtcgtatccgtggctgaggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttggataaccagaa

SEQ ID NO: 44 - DNA, 5188 bp, artificial sequence
  plasmid VN516
gcaataaaaaatcaaatcggatttcactatataatctcactttatctaagatgaatccgatggaagcatcctgttttctctcaatttttttatctaaaacccagcgttcgatgcttctttgagcgaacgatcaaaaataagtgccttcccatcaaaaaaatattgacaacataaaaaactttgtgttatacttgtaacgctacatggagattaactcaatctagctagagaggctttacactttatgcttccggctcgtataatgtgtggaattgtgagcggataacaatttcacacaggaaacagctatgaccatgattacggattcactggaactctagaccaaagagaggacacaatgcagggttctgtgacagagtttctaaaaccgcgcctggttgatatcgagcaagtgagttcgacgcacgccaaggtgacccttgagcctttagagcgtggctttggccatactctgggtaacgcactgcgccgtattctgctctcatcgatgccgggttgcgcggtgaccgaggttgagattgatggtgtactacatgagtacagcaccaaagaaggcgttcaggaagatatcctggaaatcctgctcaacctgaaagggctggcggtgagagttcagggcaaagatgaagttattcttaccttgaataaatctggcattggccctgtgactgcagccgatatcacccacgacggtgatgtcgaaatcgtcaagccgcagcacgtgatctgccacctgaccgatgagaacgcgtctattagcatgcgtatcaaagttcagcgcggtcgtggttatgtgccggcttctacccgaattcattcggaagaagatgagcgcccaatcggccgtctgctggtcgacgcatgctacagccctgtggagcgtattgcctacaatgttgaagcagcgcgtgtagaacagcgtaccgacctggacaagctggtcatcgaaatggaaaccaacggcacaatcgatcctgaagaggcgattcgtcgtgcggcaaccattctggctgaacaactggaagctttcgttgacttacgtgatgtacgtcagcctgaagtgaaagaagagaaaccagaggcggccgcaggcgcgatgggcaccatcatgccgaaagatatccagctggcgcgtcgtatccgtggcgcgggcggccgtaccctgtacggctttggctgaggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttggataaccagaa

SEQ ID NO: 45 - DNA, 5179 bp, artificial sequence
  plasmid VN517
gcaataaaaaatcaaatcggatttcactatataatctcactttatctaagatgaatccgatggaagcatcctgttttctctcaatttttttatctaaaacccagcgttcgatgcttctttgagcgaacgatcaaaaataagtgccttcccatcaaaaaaatattgacaacataaaaaactttgtgttatacttgtaacgctacatggagattaactcaatctagctagagaggctttacactttatgcttccggctcgtataatgtgtggaattgtgagcggataacaatttcacacaggaaacagctatgaccatgattacggattcactggaactctagaccaaagagaggacacaatgcagggttctgtgacagagtttctaaaaccgcgcctggttgatatcgagcaagtgagttcgacgcacgccaaggtgacccttgagcctttagagcgtggctttggccatactctgggtaacgcactgcgccgtattctgctctcatcgatgccgggttgcgcggtgaccgaggttgagattgatggtgtactacatgagtacagcaccaaagaaggcgttcaggaagatatcctggaaatcctgctcaacctgaaagggctggcggtgagagttcagggcaaagatgaagttattcttaccttgaataaatctggcattggccctgtgactgcagccgatatcacccacgacggtgatgtcgaaatcgtcaagccgcagcacgtgatctgccacctgaccgatgagaacgcgtctattagcatgcgtatcaaagttcagcgcggtcgtggttatgtgccggcttctacccgaattcattcggaagaagatgagcgcccaatcggccgtctgctggtcgacgcatgctacagccctgtggagcgtattgcctacaatgttgaagcagcgcgtgtagaacagcgtaccgacctggacaagctggtcatcgaaatggaaaccaacggcacaatcgatcctgaagaggcgattcgtcgtgcggcaaccattctggctgaacaactggaagctttcgttgacttacgtgatgtacgtcagcctgaagtgaaagaagagaaaccagaggcggccgcagcaagcaccgaacgcaaatgggcagaactggcacgtcgtattcgtggtgcaggcggtgttaccctcaatggttttggttgaggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttggataaccagaa

SEQ ID NO: 46 - DNA, 5179 bp, artificial sequence
  plasmid VN518
gcaataaaaaatcaaatcggatttcactatataatctcactttatctaagatgaatccgatggaagcatcctgttttctctcaatttttttatctaaaacccagcgttcgatgcttctttgagcgaacgatcaaaaataagtgccttcccatcaaaaaaatattgacaacataaaaaactttgtgttatacttgtaacgctacatggagattaactcaatctagctagagaggctttacactttatgcttccggctcgtataatgtgtggaattgtgagcggataacaatttcacacaggaaacagctatgaccatgattacggattcactggaactctagaccaaagagaggacacaatgcagggttctgtgacagagtttctaaaaccgcgcctggttgatatcgagcaagtgagttcgacgcacgccaaggtgacccttgagcctttagagcgtggctttggccatactctgggtaacgcactgcgccgtattctgctctcatcgatgccgggttgcgcggtgaccgaggttgagattgatggtgtactacatgagtacagcaccaaagaaggcgttcaggaagatatcctggaaatcctgctcaacctgaaagggctggcggtgagagttcagggcaaagatgaagttattcttaccttgaataaatctggcattggccctgtgactgcagccgatatcacccacgacggtgatgtcgaaatcgtcaagccgcagcacgtgatctgccacctgaccgatgagaacgcgtctattagcatgcgtatcaaagttcagcgcggtcgtggttatgtgccggcttctacccgaattcattcggaagaagatgagcgcccaatcggccgtctgctggtcgacgcatgctacagccctgtggagcgtattgcctacaatgttgaagcagcgcgtgtagaacagcgtaccgacctggacaagctggtcatcgaaatggaaaccaacggcacaatcgatcctgaagaggcgattcgtcgtgcggcaaccattctggctgaacaactggaagctttcgttgacttacgtgatgtacgtcagcctgaagtgaaagaagagaaaccagaggcggccgcagcaagcaccgaagaaaaatgggcacgtctggcacgtcgtattgcaggcgcaggcggtgttaccctggatggttttggttgaggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttggataaccagaa

SEQ ID NO: 47 - DNA, 5179 bp, artificial sequence
  plasmid VN519
gcaataaaaaatcaaatcggatttcactatataatctcactttatctaagatgaatccgatggaagcatcctgttttctctcaatttttttatctaaaacccagcgttcgatgcttctttgagcgaacgatcaaaaataagtgccttcccatcaaaaaaatattgacaacataaaaaactttgtgttatacttgtaacgctacatggagattaactcaatctagctagagaggctttacactttatgcttccggctcgtataatgtgtggaattgtgagcggataacaatttcacacaggaaacagctatgaccatgattacggattcactggaactctagaccaaagagaggacacaatgcagggttctgtgacagagtttctaaaaccgcgcctggttgatatcgagcaagtgagttcgacgcacgccaaggtgacccttgagcctttagagcgtggctttggccatactctgggtaacgcactgcgccgtattctgctctcatcgatgccgggttgcgcggtgaccgaggttgagattgatggtgtactacatgagtacagcaccaaagaaggcgttcaggaagatatcctggaaatcctgctcaacctgaaagggctggcggtgagagttcagggcaaagatgaagttattcttaccttgaataaatctggcattggccctgtgactgcagccgatatcacccacgacggtgatgtcgaaatcgtcaagccgcagcacgtgatctgccacctgaccgatgagaacgcgtctattagcatgcgtatcaaagttcagcgcggtcgtggttatgtgccggcttctacccgaattcattcggaagaagatgagcgcccaatcggccgtctgctggtcgacgcatgctacagccctgtggagcgtattgcctacaatgttgaagcagcgcgtgtagaacagcgtaccgacctggacaagctggtcatcgaaatggaaaccaacggcacaatcgatcctgaagaggcgattcgtcgtgcggcaaccattctggctgaacaactggaagctttcgttgacttacgtgatgtacgtcagcctgaagtgaaagaagagaaaccagaggcggccgcagcaagcaccgaaagaaaatgggcagaagcagcacgtcgtgcacgtggtgccggtggtgttaccctcaatggtgcaggttgaggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttggataaccagaa

SEQ ID NO: 48 - DNA, 7064 bp, artificial sequence
  plasmid VN750
ggcagcattcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggtgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccagagcctgataaaaacggttagcgcttcgttaatacagatgtaggtgttccacagggtagccagcagcatcctgcgatgcagatctgtaaggtaagagctcttatcagttatcccaattgatatggaagcgggtaacacgcggattggatgccagaatgttgcgttgcagctgactaaaatccggtttcatcggcggattttcacgcagttccgggttcaggtattcattgttaacgtaataacccacgcggatgaattcctggccgtgatacgtgcaggtaatcagaaccaccgtgacaccaactgcgtcggtttccggaatcagcgacgggttcggagcatctgcttggaaaacaaacatatgacgacctgccgggaccgggcccaccaggactgagtccaggatctgatcaaattcttcagattccgcgctgcccacatagataattttccattccaggtcatcggccagcgcttcggaacattcaaacgagatttcaaagcggaacgggctatggaacggggacggattttccagcactgccacattcagcaccgagactttcgccattgcggtaccgccaaacgtctcttcaggccactgactagcgataactttccccacaacggaacaactctcattgcatgggatcattgggtactgtgggtttagtggttgtaaaaacacctgaccgctatccctgatcagtttcttgaaggtaaactcatcacccccaagtctggctatgcagaaatcacctggctcaacagcctgctcagggtcaacgagaattaacattccgtcaggaaagcttggcttggagcctgttggtgcggtcatggaattaccttcaacctcaagccagaatgcagaatcactggcttttttggttgtgcttacccatctctccgcatcacctttggtaaaggttctaagctcaggtgagaacatccctgcctgaacatgagaaaaaacagggtactcatactcacttctaagtgacggctgcatactaaccgcttcatacatctcgtagatttctctggcgattgaagggctaaattcttcaacgctaactttgagaatttttgcaagcaatgcggcgttataagcatttaatgcattgatgccattaaataaagcaccaacgcctgactgccccatccccatcttgtctgcgacagacggctgggataagccaagttcatttttctttttttcataaattgctttaaggcgacgtgcgtcctcaagctgctcttgtgttaatggtttcttttttgtgctcatacgctgtttcctgtgtgaaattgttatccgctcacaattccacacattatacgagccggaagcataaagtgtaaagcctggggtgcctaatgagtgagaattagcttatcgatgataagctgtcaaacatgagaattacaacttatatcgtatggggctgacttcaggtgctacatttgaagagataaattgcactgaaatctagaatgtctccagaccgctagtagacgagtccatgtgctggcgttcaaatttcgcagcagcggtttctttaccagactcgagagcgaggctcttcaagctcgcaaaattgctcttcaagctcgcaaaaaaccccgcccctgacagggcggggttttttcgcgttcgatcccgcgaaatttaattaaccaacaccgccagagatacattaggcaccccaggcttgacactttatgcttccggctcggatactgtgtggaattgaaagaggaacgtacgatggtcagtaaaggcgaagagttgtttacgggtgtagtaccgattttagtggaactcgatggagatgtgaatgggcataaattttcggtttcaggtgaaggtgaaggagatgccacctatggcaaactgacgctgaagttcatctgtacgacaggcaaactgcctgttccgtggccaacactggttactacgctgacctatggcgttcaatgcttttcgcgttatccggaccacatgaaacagcatgacttctttaaatccgcgatgcctgaaggttacgtacaagaacgcaccatcttcttcaaagatgacgggaactacaaaacccgtgcagaggtcaaatttgagggtgacacccttgtgaatcggattgagctgaaaggcattgactttaaagaagatggcaacattctgggtcataaactggagtacaactacaacagccataacgtgtatatcatggcggacaaacagaagaatgggatcaaagtgaatttcaagattcgccacaacattgaagatggtagtgtgcagttagccgaccactatcagcagaataccccgattggcgatggcccagtcttgctgccggataatcattaccttagcacccaatctgcgttaagcaaagatcccaacgaaaagcgcgatcacatggtcctcctggaatttgtgactgctgcaggcatcactttgggtatggatgaactgtataagtaaacgcgttaaacaggaaacagaagtccaaatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcagatctaaataataaaaaagccggattaataatctggctttttatattctctctctagtatataaacgcagaaaggcccacccgaaggtgagccagtgtgacgaacccccgcctcagagtgggaaagtctagaaatattttatctgattaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccaggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgcgaacgatcaaaaataagtgccttcccatcaaaaaaatattgacaacataaaaaactttgtgttatacttgtaacgctacatggagattaactcaatctagctagagaggctttacactttatgcttccggctcgtataatgtgtggaattgtgagcggataacaatttcacacaggaaacagctatgaccatgattacggattcactggaactctagaccaaagagaggacacaatgcagggttctgtgacagagtttctaaaaccgcgcctggttgatatcgagcaagtgagttcgacgcacgccaaggtgacccttgagcctttagagcgtggctttggccatactctgggtaacgcactgcgccgtattctgctctcatcgatgccgggttgcgcggtgaccgaggttgagattgatggtgtactacatgagtacagcaccaaagaaggcgttcaggaagatatcctggaaatcctgctcaacctgaaagggctggcggtgagagttcagggcaaagatgaagttattcttaccttgaataaatctggcattggccctgtgactgcagccgatatcacccacgacggtgatgtcgaaatcgtcaagccgcagcacgtgatctgccacctgaccgatgagaacgcgtctattagcatgcgtatcaaagttcagcgcggtcgtggttatgtgccggcttctacccgaattcattcggaagaagatgagcgcccaatcggccgtctgctggtcgacgcatgctacagccctgtggagcgtattgcctacaatgttgaagcagcgcgtgtagaacagcgtaccgacctggacaagctggtcatcgaaatggaaaccaacggcacaatcgatcctgaagaggcgattcgtcgtgcggcaaccattctggctgaacaactggaagctttcgttgacttacgtgatgtacgtcagcctgaagtgaaagaagagaaaccagaggcggccgcaggcgcgatgggcaccatcatgccgaaagatatccagctggcgcgtcgtatccgtggctgaggatcctctacgccggacgcactcggttccaaaaaaaaaaaaaaagacgctgaaaagcgtcttttttttttttggtccccactggagcacctcaaaaacaccatcatacactaatcagtaagtt

SEQ ID NO: 49 - DNA, 7094 bp, artificial sequence
  plasmid VN751
ggcagcattcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggtgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccagagcctgataaaaacggttagcgcttcgttaatacagatgtaggtgttccacagggtagccagcagcatcctgcgatgcagatctgtaaggtaagagctcttatcagttatcccaattgatatggaagcgggtaacacgcggattggatgccagaatgttgcgttgcagctgactaaaatccggtttcatcggcggattttcacgcagttccgggttcaggtattcattgttaacgtaataacccacgcggatgaattcctggccgtgatacgtgcaggtaatcagaaccaccgtgacaccaactgcgtcggtttccggaatcagcgacgggttcggagcatctgcttggaaaacaaacatatgacgacctgccgggaccgggcccaccaggactgagtccaggatctgatcaaattcttcagattccgcgctgcccacatagataattttccattccaggtcatcggccagcgcttcggaacattcaaacgagatttcaaagcggaacgggctatggaacggggacggattttccagcactgccacattcagcaccgagactttcgccattgcggtaccgccaaacgtctcttcaggccactgactagcgataactttccccacaacggaacaactctcattgcatgggatcattgggtactgtgggtttagtggttgtaaaaacacctgaccgctatccctgatcagtttcttgaaggtaaactcatcacccccaagtctggctatgcagaaatcacctggctcaacagcctgctcagggtcaacgagaattaacattccgtcaggaaagcttggcttggagcctgttggtgcggtcatggaattaccttcaacctcaagccagaatgcagaatcactggcttttttggttgtgcttacccatctctccgcatcacctttggtaaaggttctaagctcaggtgagaacatccctgcctgaacatgagaaaaaacagggtactcatactcacttctaagtgacggctgcatactaaccgcttcatacatctcgtagatttctctggcgattgaagggctaaattcttcaacgctaactttgagaatttttgcaagcaatgcggcgttataagcatttaatgcattgatgccattaaataaagcaccaacgcctgactgccccatccccatcttgtctgcgacagacggctgggataagccaagttcatttttctttttttcataaattgctttaaggcgacgtgcgtcctcaagctgctcttgtgttaatggtttcttttttgtgctcatacgctgtttcctgtgtgaaattgttatccgctcacaattccacacattatacgagccggaagcataaagtgtaaagcctggggtgcctaatgagtgagaattagcttatcgatgataagctgtcaaacatgagaattacaacttatatcgtatggggctgacttcaggtgctacatttgaagagataaattgcactgaaatctagaatgtctccagaccgctagtagacgagtccatgtgctggcgttcaaatttcgcagcagcggtttctttaccagactcgagagcgaggctcttcaagctcgcaaaattgctcttcaagctcgcaaaaaaccccgcccctgacagggcggggttttttcgcgttcgatcccgcgaaatttaattaaccaacaccgccagagatacattaggcaccccaggcttgacactttatgcttccggctcggatactgtgtggaattgaaagaggaacgtacgatggtcagtaaaggcgaagagttgtttacgggtgtagtaccgattttagtggaactcgatggagatgtgaatgggcataaattttcggtttcaggtgaaggtgaaggagatgccacctatggcaaactgacgctgaagttcatctgtacgacaggcaaactgcctgttccgtggccaacactggttactacgctgacctatggcgttcaatgcttttcgcgttatccggaccacatgaaacagcatgacttctttaaatccgcgatgcctgaaggttacgtacaagaacgcaccatcttcttcaaagatgacgggaactacaaaacccgtgcagaggtcaaatttgagggtgacacccttgtgaatcggattgagctgaaaggcattgactttaaagaagatggcaacattctgggtcataaactggagtacaactacaacagccataacgtgtatatcatggcggacaaacagaagaatgggatcaaagtgaatttcaagattcgccacaacattgaagatggtagtgtgcagttagccgaccactatcagcagaataccccgattggcgatggcccagtcttgctgccggataatcattaccttagcacccaatctgcgttaagcaaagatcccaacgaaaagcgcgatcacatggtcctcctggaatttgtgactgctgcaggcatcactttgggtatggatgaactgtataagtaaacgcgttaaacaggaaacagaagtccaaatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcagatctaaataataaaaaagccggattaataatctggctttttatattctctctctagtatataaacgcagaaaggcccacccgaaggtgagccagtgtgacgaacccccgcctcagagtgggaaagtctagaaatattttatctgattaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccaggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgcgaacgatcaaaaataagtgccttcccatcaaaaaaatattgacaacataaaaaactttgtgttatacttgtaacgctacatggagattaactcaatctagctagagaggctttacactttatgcttccggctcgtataatgtgtggaattgtgagcggataacaatttcacacaggaaacagctatgaccatgattacggattcactggaactctagaccaaagagaggacacaatgcagggttctgtgacagagtttctaaaaccgcgcctggttgatatcgagcaagtgagttcgacgcacgccaaggtgacccttgagcctttagagcgtggctttggccatactctgggtaacgcactgcgccgtattctgctctcatcgatgccgggttgcgcggtgaccgaggttgagattgatggtgtactacatgagtacagcaccaaagaaggcgttcaggaagatatcctggaaatcctgctcaacctgaaagggctggcggtgagagttcagggcaaagatgaagttattcttaccttgaataaatctggcattggccctgtgactgcagccgatatcacccacgacggtgatgtcgaaatcgtcaagccgcagcacgtgatctgccacctgaccgatgagaacgcgtctattagcatgcgtatcaaagttcagcgcggtcgtggttatgtgccggcttctacccgaattcattcggaagaagatgagcgcccaatcggccgtctgctggtcgacgcatgctacagccctgtggagcgtattgcctacaatgttgaagcagcgcgtgtagaacagcgtaccgacctggacaagctggtcatcgaaatggaaaccaacggcacaatcgatcctgaagaggcgattcgtcgtgcggcaaccattctggctgaacaactggaagctttcgttgacttacgtgatgtacgtcagcctgaagtgaaagaagagaaaccagaggcggccgcaggcgcgatgggcaccatcatgccgaaagatatccagctggcgcgtcgtatccgtggcgcgggcggccgtaccctgtacggctttggctgaggatcctctacgccggacgcactcggttccaaaaaaaaaaaaaaagacgctgaaaagcgtcttttttttttttggtccccactggagcacctcaaaaacaccatcatacactaatcagtaagtt

SEQ ID NO: 50 - DNA, 7085 bp, artificial sequence
  plasmid VN752
ggcagcattcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggtgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccagagcctgataaaaacggttagcgcttcgttaatacagatgtaggtgttccacagggtagccagcagcatcctgcgatgcagatctgtaaggtaagagctcttatcagttatcccaattgatatggaagcgggtaacacgcggattggatgccagaatgttgcgttgcagctgactaaaatccggtttcatcggcggattttcacgcagttccgggttcaggtattcattgttaacgtaataacccacgcggatgaattcctggccgtgatacgtgcaggtaatcagaaccaccgtgacaccaactgcgtcggtttccggaatcagcgacgggttcggagcatctgcttggaaaacaaacatatgacgacctgccgggaccgggcccaccaggactgagtccaggatctgatcaaattcttcagattccgcgctgcccacatagataattttccattccaggtcatcggccagcgcttcggaacattcaaacgagatttcaaagcggaacgggctatggaacggggacggattttccagcactgccacattcagcaccgagactttcgccattgcggtaccgccaaacgtctcttcaggccactgactagcgataactttccccacaacggaacaactctcattgcatgggatcattgggtactgtgggtttagtggttgtaaaaacacctgaccgctatccctgatcagtttcttgaaggtaaactcatcacccccaagtctggctatgcagaaatcacctggctcaacagcctgctcagggtcaacgagaattaacattccgtcaggaaagcttggcttggagcctgttggtgcggtcatggaattaccttcaacctcaagccagaatgcagaatcactggcttttttggttgtgcttacccatctctccgcatcacctttggtaaaggttctaagctcaggtgagaacatccctgcctgaacatgagaaaaaacagggtactcatactcacttctaagtgacggctgcatactaaccgcttcatacatctcgtagatttctctggcgattgaagggctaaattcttcaacgctaactttgagaatttttgcaagcaatgcggcgttataagcatttaatgcattgatgccattaaataaagcaccaacgcctgactgccccatccccatcttgtctgcgacagacggctgggataagccaagttcatttttctttttttcataaattgctttaaggcgacgtgcgtcctcaagctgctcttgtgttaatggtttcttttttgtgctcatacgctgtttcctgtgtgaaattgttatccgctcacaattccacacattatacgagccggaagcataaagtgtaaagcctggggtgcctaatgagtgagaattagcttatcgatgataagctgtcaaacatgagaattacaacttatatcgtatggggctgacttcaggtgctacatttgaagagataaattgcactgaaatctagaatgtctccagaccgctagtagacgagtccatgtgctggcgttcaaatttcgcagcagcggtttctttaccagactcgagagcgaggctcttcaagctcgcaaaattgctcttcaagctcgcaaaaaaccccgcccctgacagggcggggttttttcgcgttcgatcccgcgaaatttaattaaccaacaccgccagagatacattaggcaccccaggcttgacactttatgcttccggctcggatactgtgtggaattgaaagaggaacgtacgatggtcagtaaaggcgaagagttgtttacgggtgtagtaccgattttagtggaactcgatggagatgtgaatgggcataaattttcggtttcaggtgaaggtgaaggagatgccacctatggcaaactgacgctgaagttcatctgtacgacaggcaaactgcctgttccgtggccaacactggttactacgctgacctatggcgttcaatgcttttcgcgttatccggaccacatgaaacagcatgacttctttaaatccgcgatgcctgaaggttacgtacaagaacgcaccatcttcttcaaagatgacgggaactacaaaacccgtgcagaggtcaaatttgagggtgacacccttgtgaatcggattgagctgaaaggcattgactttaaagaagatggcaacattctgggtcataaactggagtacaactacaacagccataacgtgtatatcatggcggacaaacagaagaatgggatcaaagtgaatttcaagattcgccacaacattgaagatggtagtgtgcagttagccgaccactatcagcagaataccccgattggcgatggcccagtcttgctgccggataatcattaccttagcacccaatctgcgttaagcaaagatcccaacgaaaagcgcgatcacatggtcctcctggaatttgtgactgctgcaggcatcactttgggtatggatgaactgtataagtaaacgcgttaaacaggaaacagaagtccaaatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcagatctaaataataaaaaagccggattaataatctggctttttatattctctctctagtatataaacgcagaaaggcccacccgaaggtgagccagtgtgacgaacccccgcctcagagtgggaaagtctagaaatattttatctgattaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccaggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgcgaacgatcaaaaataagtgccttcccatcaaaaaaatattgacaacataaaaaactttgtgttatacttgtaacgctacatggagattaactcaatctagctagagaggctttacactttatgcttccggctcgtataatgtgtggaattgtgagcggataacaatttcacacaggaaacagctatgaccatgattacggattcactggaactctagaccaaagagaggacacaatgcagggttctgtgacagagtttctaaaaccgcgcctggttgatatcgagcaagtgagttcgacgcacgccaaggtgacccttgagcctttagagcgtggctttggccatactctgggtaacgcactgcgccgtattctgctctcatcgatgccgggttgcgcggtgaccgaggttgagattgatggtgtactacatgagtacagcaccaaagaaggcgttcaggaagatatcctggaaatcctgctcaacctgaaagggctggcggtgagagttcagggcaaagatgaagttattcttaccttgaataaatctggcattggccctgtgactgcagccgatatcacccacgacggtgatgtcgaaatcgtcaagccgcagcacgtgatctgccacctgaccgatgagaacgcgtctattagcatgcgtatcaaagttcagcgcggtcgtggttatgtgccggcttctacccgaattcattcggaagaagatgagcgcccaatcggccgtctgctggtcgacgcatgctacagccctgtggagcgtattgcctacaatgttgaagcagcgcgtgtagaacagcgtaccgacctggacaagctggtcatcgaaatggaaaccaacggcacaatcgatcctgaagaggcgattcgtcgtgcggcaaccattctggctgaacaactggaagctttcgttgacttacgtgatgtacgtcagcctgaagtgaaagaagagaaaccagaggcggccgcagcaagcaccgaacgcaaatgggcagaactggcacgtcgtattcgtggtgcaggcggtgttaccctcaatggttttggttgaggatcctctacgccggacgcactcggttccaaaaaaaaaaaaaaagacgctgaaaagcgtcttttttttttttggtccccactggagcacctcaaaaacaccatcatacactaatcagtaagtt

SEQ ID NO: 51 - DNA, 7085 bp, artificial sequence
  plasmid VN753
ggcagcattcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggtgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccagagcctgataaaaacggttagcgcttcgttaatacagatgtaggtgttccacagggtagccagcagcatcctgcgatgcagatctgtaaggtaagagctcttatcagttatcccaattgatatggaagcgggtaacacgcggattggatgccagaatgttgcgttgcagctgactaaaatccggtttcatcggcggattttcacgcagttccgggttcaggtattcattgttaacgtaataacccacgcggatgaattcctggccgtgatacgtgcaggtaatcagaaccaccgtgacaccaactgcgtcggtttccggaatcagcgacgggttcggagcatctgcttggaaaacaaacatatgacgacctgccgggaccgggcccaccaggactgagtccaggatctgatcaaattcttcagattccgcgctgcccacatagataattttccattccaggtcatcggccagcgcttcggaacattcaaacgagatttcaaagcggaacgggctatggaacggggacggattttccagcactgccacattcagcaccgagactttcgccattgcggtaccgccaaacgtctcttcaggccactgactagcgataactttccccacaacggaacaactctcattgcatgggatcattgggtactgtgggtttagtggttgtaaaaacacctgaccgctatccctgatcagtttcttgaaggtaaactcatcacccccaagtctggctatgcagaaatcacctggctcaacagcctgctcagggtcaacgagaattaacattccgtcaggaaagcttggcttggagcctgttggtgcggtcatggaattaccttcaacctcaagccagaatgcagaatcactggcttttttggttgtgcttacccatctctccgcatcacctttggtaaaggttctaagctcaggtgagaacatccctgcctgaacatgagaaaaaacagggtactcatactcacttctaagtgacggctgcatactaaccgcttcatacatctcgtagatttctctggcgattgaagggctaaattcttcaacgctaactttgagaatttttgcaagcaatgcggcgttataagcatttaatgcattgatgccattaaataaagcaccaacgcctgactgccccatccccatcttgtctgcgacagacggctgggataagccaagttcatttttctttttttcataaattgctttaaggcgacgtgcgtcctcaagctgctcttgtgttaatggtttcttttttgtgctcatacgctgtttcctgtgtgaaattgttatccgctcacaattccacacattatacgagccggaagcataaagtgtaaagcctggggtgcctaatgagtgagaattagcttatcgatgataagctgtcaaacatgagaattacaacttatatcgtatggggctgacttcaggtgctacatttgaagagataaattgcactgaaatctagaatgtctccagaccgctagtagacgagtccatgtgctggcgttcaaatttcgcagcagcggtttctttaccagactcgagagcgaggctcttcaagctcgcaaaattgctcttcaagctcgcaaaaaaccccgcccctgacagggcggggttttttcgcgttcgatcccgcgaaatttaattaaccaacaccgccagagatacattaggcaccccaggcttgacactttatgcttccggctcggatactgtgtggaattgaaagaggaacgtacgatggtcagtaaaggcgaagagttgtttacgggtgtagtaccgattttagtggaactcgatggagatgtgaatgggcataaattttcggtttcaggtgaaggtgaaggagatgccacctatggcaaactgacgctgaagttcatctgtacgacaggcaaactgcctgttccgtggccaacactggttactacgctgacctatggcgttcaatgcttttcgcgttatccggaccacatgaaacagcatgacttctttaaatccgcgatgcctgaaggttacgtacaagaacgcaccatcttcttcaaagatgacgggaactacaaaacccgtgcagaggtcaaatttgagggtgacacccttgtgaatcggattgagctgaaaggcattgactttaaagaagatggcaacattctgggtcataaactggagtacaactacaacagccataacgtgtatatcatggcggacaaacagaagaatgggatcaaagtgaatttcaagattcgccacaacattgaagatggtagtgtgcagttagccgaccactatcagcagaataccccgattggcgatggcccagtcttgctgccggataatcattaccttagcacccaatctgcgttaagcaaagatcccaacgaaaagcgcgatcacatggtcctcctggaatttgtgactgctgcaggcatcactttgggtatggatgaactgtataagtaaacgcgttaaacaggaaacagaagtccaaatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcagatctaaataataaaaaagccggattaataatctggctttttatattctctctctagtatataaacgcagaaaggcccacccgaaggtgagccagtgtgacgaacccccgcctcagagtgggaaagtctagaaatattttatctgattaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccaggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgcgaacgatcaaaaataagtgccttcccatcaaaaaaatattgacaacataaaaaactttgtgttatacttgtaacgctacatggagattaactcaatctagctagagaggctttacactttatgcttccggctcgtataatgtgtggaattgtgagcggataacaatttcacacaggaaacagctatgaccatgattacggattcactggaactctagaccaaagagaggacacaatgcagggttctgtgacagagtttctaaaaccgcgcctggttgatatcgagcaagtgagttcgacgcacgccaaggtgacccttgagcctttagagcgtggctttggccatactctgggtaacgcactgcgccgtattctgctctcatcgatgccgggttgcgcggtgaccgaggttgagattgatggtgtactacatgagtacagcaccaaagaaggcgttcaggaagatatcctggaaatcctgctcaacctgaaagggctggcggtgagagttcagggcaaagatgaagttattcttaccttgaataaatctggcattggccctgtgactgcagccgatatcacccacgacggtgatgtcgaaatcgtcaagccgcagcacgtgatctgccacctgaccgatgagaacgcgtctattagcatgcgtatcaaagttcagcgcggtcgtggttatgtgccggcttctacccgaattcattcggaagaagatgagcgcccaatcggccgtctgctggtcgacgcatgctacagccctgtggagcgtattgcctacaatgttgaagcagcgcgtgtagaacagcgtaccgacctggacaagctggtcatcgaaatggaaaccaacggcacaatcgatcctgaagaggcgattcgtcgtgcggcaaccattctggctgaacaactggaagctttcgttgacttacgtgatgtacgtcagcctgaagtgaaagaagagaaaccagaggcggccgcagcaagcaccgaagaaaaatgggcacgtctggcacgtcgtattgcaggcgcaggcggtgttaccctggatggttttggttgaggatcctctacgccggacgcactcggttccaaaaaaaaaaaaaaagacgctgaaaagcgtcttttttttttttggtccccactggagcacctcaaaaacaccatcatacactaatcagtaagtt

SEQ ID NO: 52 - DNA, 7085 bp, artificial sequence
  plasmid VN754
ggcagcattcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggtgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccagagcctgataaaaacggttagcgcttcgttaatacagatgtaggtgttccacagggtagccagcagcatcctgcgatgcagatctgtaaggtaagagctcttatcagttatcccaattgatatggaagcgggtaacacgcggattggatgccagaatgttgcgttgcagctgactaaaatccggtttcatcggcggattttcacgcagttccgggttcaggtattcattgttaacgtaataacccacgcggatgaattcctggccgtgatacgtgcaggtaatcagaaccaccgtgacaccaactgcgtcggtttccggaatcagcgacgggttcggagcatctgcttggaaaacaaacatatgacgacctgccgggaccgggcccaccaggactgagtccaggatctgatcaaattcttcagattccgcgctgcccacatagataattttccattccaggtcatcggccagcgcttcggaacattcaaacgagatttcaaagcggaacgggctatggaacggggacggattttccagcactgccacattcagcaccgagactttcgccattgcggtaccgccaaacgtctcttcaggccactgactagcgataactttccccacaacggaacaactctcattgcatgggatcattgggtactgtgggtttagtggttgtaaaaacacctgaccgctatccctgatcagtttcttgaaggtaaactcatcacccccaagtctggctatgcagaaatcacctggctcaacagcctgctcagggtcaacgagaattaacattccgtcaggaaagcttggcttggagcctgttggtgcggtcatggaattaccttcaacctcaagccagaatgcagaatcactggcttttttggttgtgcttacccatctctccgcatcacctttggtaaaggttctaagctcaggtgagaacatccctgcctgaacatgagaaaaaacagggtactcatactcacttctaagtgacggctgcatactaaccgcttcatacatctcgtagatttctctggcgattgaagggctaaattcttcaacgctaactttgagaatttttgcaagcaatgcggcgttataagcatttaatgcattgatgccattaaataaagcaccaacgcctgactgccccatccccatcttgtctgcgacagacggctgggataagccaagttcatttttctttttttcataaattgctttaaggcgacgtgcgtcctcaagctgctcttgtgttaatggtttcttttttgtgctcatacgctgtttcctgtgtgaaattgttatccgctcacaattccacacattatacgagccggaagcataaagtgtaaagcctggggtgcctaatgagtgagaattagcttatcgatgataagctgtcaaacatgagaattacaacttatatcgtatggggctgacttcaggtgctacatttgaagagataaattgcactgaaatctagaatgtctccagaccgctagtagacgagtccatgtgctggcgttcaaatttcgcagcagcggtttctttaccagactcgagagcgaggctcttcaagctcgcaaaattgctcttcaagctcgcaaaaaaccccgcccctgacagggcggggttttttcgcgttcgatcccgcgaaatttaattaaccaacaccgccagagatacattaggcaccccaggcttgacactttatgcttccggctcggatactgtgtggaattgaaagaggaacgtacgatggtcagtaaaggcgaagagttgtttacgggtgtagtaccgattttagtggaactcgatggagatgtgaatgggcataaattttcggtttcaggtgaaggtgaaggagatgccacctatggcaaactgacgctgaagttcatctgtacgacaggcaaactgcctgttccgtggccaacactggttactacgctgacctatggcgttcaatgcttttcgcgttatccggaccacatgaaacagcatgacttctttaaatccgcgatgcctgaaggttacgtacaagaacgcaccatcttcttcaaagatgacgggaactacaaaacccgtgcagaggtcaaatttgagggtgacacccttgtgaatcggattgagctgaaaggcattgactttaaagaagatggcaacattctgggtcataaactggagtacaactacaacagccataacgtgtatatcatggcggacaaacagaagaatgggatcaaagtgaatttcaagattcgccacaacattgaagatggtagtgtgcagttagccgaccactatcagcagaataccccgattggcgatggcccagtcttgctgccggataatcattaccttagcacccaatctgcgttaagcaaagatcccaacgaaaagcgcgatcacatggtcctcctggaatttgtgactgctgcaggcatcactttgggtatggatgaactgtataagtaaacgcgttaaacaggaaacagaagtccaaatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcagatctaaataataaaaaagccggattaataatctggctttttatattctctctctagtatataaacgcagaaaggcccacccgaaggtgagccagtgtgacgaacccccgcctcagagtgggaaagtctagaaatattttatctgattaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccaggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgcgaacgatcaaaaataagtgccttcccatcaaaaaaatattgacaacataaaaaactttgtgttatacttgtaacgctacatggagattaactcaatctagctagagaggctttacactttatgcttccggctcgtataatgtgtggaattgtgagcggataacaatttcacacaggaaacagctatgaccatgattacggattcactggaactctagaccaaagagaggacacaatgcagggttctgtgacagagtttctaaaaccgcgcctggttgatatcgagcaagtgagttcgacgcacgccaaggtgacccttgagcctttagagcgtggctttggccatactctgggtaacgcactgcgccgtattctgctctcatcgatgccgggttgcgcggtgaccgaggttgagattgatggtgtactacatgagtacagcaccaaagaaggcgttcaggaagatatcctggaaatcctgctcaacctgaaagggctggcggtgagagttcagggcaaagatgaagttattcttaccttgaataaatctggcattggccctgtgactgcagccgatatcacccacgacggtgatgtcgaaatcgtcaagccgcagcacgtgatctgccacctgaccgatgagaacgcgtctattagcatgcgtatcaaagttcagcgcggtcgtggttatgtgccggcttctacccgaattcattcggaagaagatgagcgcccaatcggccgtctgctggtcgacgcatgctacagccctgtggagcgtattgcctacaatgttgaagcagcgcgtgtagaacagcgtaccgacctggacaagctggtcatcgaaatggaaaccaacggcacaatcgatcctgaagaggcgattcgtcgtgcggcaaccattctggctgaacaactggaagctttcgttgacttacgtgatgtacgtcagcctgaagtgaaagaagagaaaccagaggcggccgcagcaagcaccgaaagaaaatgggcagaagcagcacgtcgtgcacgtggtgccggtggtgttaccctcaatggtgcaggttgaggatcctctacgccggacgcactcggttccaaaaaaaaaaaaaaagacgctgaaaagcgtcttttttttttttggtccccactggagcacctcaaaaacaccatcatacactaatcagtaagtt

SEQ ID NO: 53 - DNA, 7533 bp, artificial sequence
  plasmid VN1197
ggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggtgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccagagcctgataaaaacggttagcgcttcgttaatacagatgtaggtgttccacagggtagccagcagcatcctgcgatgcagatctgtaaggtaagagctcttatcaagatccgttatcccaattgatatggaagcgggtaacacgcggattggatgccagaatgttgcgttgcagctgactaaaatccggtttcatcggcggattttcacgcagttccgggttcaggtattcattgttaacgtaataacccacgcggatgaattcctggccgtgatacgtgcaggtaatcagaaccaccgtgacaccaactgcgtcggtttccggaatcagcgacgggttcggagcatctgcttggaaaacaaacatatgacgacctgccgggaccgggcccaccaggacgctgtccaggatctgatcaaattcttcagattccgcgctgcccacatagataattttccattccaggtcatcggccagcgcttcggaacattcaaacgagatttcaaagcggaacgggctatggaacggggacggattttccagcactgccacattcagcaccgagactttcgccatggcggtaccgccaaacgtctcttcaggccactgactagcgataactttccccacaacggaacaactctcattgcatgggatcattgggtactgtgggtttagtggttgtaaaaacacctgaccgctatccctgatcagtttcttgaaggtaaactcatcacccccaagtctggctatgcagaaatcacctggctcaacagcctgctcagggtcaacgagaattaacattccgtcaggaaagcttggcttggagcctgttggtgcggtcatggaattaccttcaacctcaagccagaatgcagaatcactggcttttttggttgtgcttacccatctctccgcatcacctttggtaaaggttctaagctcaggtgagaacatccctgcctgaacatgagaaaaaacagggtactcatactcacttctaagtgacggctgcatactaaccgcttcatacatctcgtagatttctctggcgattgaagggctaaattcttcaacgctaactttgagaatttttgcaagcaatgcggcgttataagcatttaatgcattgatgccattaaataaagcaccaacgcctgactgccccatccccatcttgtctgcgacagacggctgggataagccaagttcatttttctttttttcataaattgctttaaggcgacgtgcgtcctcaagctgctcttgtgttaatggtttcttttttgtgctcatacgctgtttcctgtgtgaaattgttatccgctcacaattccacacattatacgagccggaagcataaagtgtaaagcctggggtgcctaatgagtgagaattagcttatcgatgataagctgtcaaacatgagaattacaacttatatcgtatggggctgacttcaggtgctacatttgaagagataaattgcactgaaatctagatgtctccagaccgctagtagacgagtccatgtgctggcgttcaaatttcgcagcagcggtttctttaccagactcgagagcgaggctcttcaagctcgcaaaattgctcttcaagctcgcaaaaaaccccgcccctgacagggcggggttttttcgcgttcgatcccgcgaaatttaattaaccaacaccgccagagatacattaggcaccccaggcttgacactttatgcttccggctcggatactgtgtggaattgaaagaggaacgtacgatgaaaacttctgaacaacgtgtaaacatcgcaactctgctgactgagaacaaaaagaaaatcgttgataaggcgtctcaggatctgtggcgtcgtcatccagatctcattgcgccgggcggcatcgcttttagtcaacgcgatcgtgcactgtgcctgcgtgactacggctggtttctgcacctgatcacgttctgtctgctggctggtgataaaggtcctattgaatccatcggtctaattagcatacgcgaaatgtacaacagcctgggtgtgccggttccggctatgatggaatctatccgttgtctgaaagaggcctccctgtcactgctggacgaggaagacgccaatgagactgcaccgtactttgactatatcatcaaggctatgtccgaattccatcatcaccatcaccattgaaagaaggagatatacatacccatgagtgtcaacttagcttcccagttgcgggaagggacgaaaaaatcccactccatggcggagaacgtcggctttgtcaaatgcttcctcaagggcgttgtcgagaaaaattcctaccgtaagctggttggcaatctctactttgtctacagtgccatggaagaggaaatggcaaaatttaaggaccatcccatcctcagccacatttacttccccgaactcaaccgcaaacaaagcctagagcaagacctgcaattctattacggctccaactggcggcaagaagtgaaaatttctgccgctggccaagcctatgtggaccgagtccggcaagtggccgctacggcccctgaattgttggtggcccattcctacacccgttacctgggggatctttccggcggtcaaattctcaagaaaattgcccaaaatgccatgaatctccacgatggtggcacagctttctatgaatttgccgacattgatgacgaaaaggcttttaaaaatacctaccgtcaagctatgaatgatctgcccattgaccaagccaccgccgaacggattgtggatgaagccaatgacgcctttgccatgaacatgaaaatgttcaacgaacttgaaggcaacctgatcaaggcgatcggcattatggtgttcaacagcctcacccgtcgccgcagtcaaggcagcaccgaagttggcctcgccacctccgaaggctgaaattcctggtccaggaatttcacatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagactagtgaattaattcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcagatctaaataataaaaaagccggattaataatctggctttttatattctctctctagtatataaacgcagaaaggcccacccgaaggtgagccagtgtgacgaacccccgcctcagagtgggaaagtctagaaatattttatctgattaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctctccgcagtcgaacgaccgagcgtagcgagtcagtgcgaacgatcaaaaataagtgccttcccatcaaaaaaatattgacaacataaaaaactttgtgttatacttgtaacgctacatggagattaactcaatctagctagagaggctttacactttatgcttccggctcgtataatgtgtggaattgtgagcggataacaatttcacacaggaaacagctatgaccatgattacggattcactggaactctagaccaaagagaggacacaatgcagggttctgtgacagagtttctaaaaccgcgcctggttgatatcgagcaagtgagttcgacgcacgccaaggtgacccttgagcctttagagcgtggctttggccatactctgggtaacgcactgcgccgtattctgctctcatcgatgccgggttgcgcggtgaccgaggttgagattgatggtgtactacatgagtacagcaccaaagaaggcgttcaggaagatatcctggaaatcctgctcaacctgaaagggctggcggtgagagttcagggcaaagatgaagttattcttaccttgaataaatctggcattggccctgtgactgcagccgatatcacccacgacggtgatgtcgaaatcgtcaagccgcagcacgtgatctgccacctgaccgatgagaacgcgtctattagcatgcgtatcaaagttcagcgcggtcgtggttatgtgccggcttctacccgaattcattcggaagaagatgagcgcccaatcggccgtctgctggtcgacgcatgctacagccctgtggagcgtattgcctacaatgttgaagcagcgcgtgtagaacagcgtaccgacctggacaagctggtcatcgaaatggaaaccaacggcacaatcgatcctgaagaggcgattcgtcgtgcggcaaccattctggctgaacaactggaagctttcgttgacttacgtgatgtacgtcagcctgaagtgaaagaagagaaaccagaggcggccgcagcaagcaccgaacgcaaatgggcagaactggcacgtcgtattcgtggtgcaggcggtgttaccctcaatggttttggttgaggatcctctacgccggacgcactcggttccaaaaaaaaaaaaaaagacgctgaaaagcgtcttttttttttttggtccccactggagcacctcaaaaacaccatcatacactaaatcagtaagtt

SEQ ID NO: 54 - DNA, 6206 bp, artificial sequence
  plasmid VN1296
ggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggtgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccagagcctgataaaaacggttagcgcttcgttaatacagatgtaggtgttccacagggtagccagcagcatcctgcgatgcagatctgtaaggtaagagctcttatcaagatccgttatcccaattgatatggaagcgggtaacacgcggattggatgccagaatgttgcgttgcagctgactaaaatccggtttcatcggcggattttcacgcagttccgggttcaggtattcattgttaacgtaataacccacgcggatgaattcctggccgtgatacgtgcaggtaatcagaaccaccgtgacaccaactgcgtcggtttccggaatcagcgacgggttcggagcatctgcttggaaaacaaacatatgacgacctgccgggaccgggcccaccaggacgctgtccaggatctgatcaaattcttcagattccgcgctgcccacatagataattttccattccaggtcatcggccagcgcttcggaacattcaaacgagatttcaaagcggaacgggctatggaacggggacggattttccagcactgccacattcagcaccgagactttcgccatggcggtaccgccaaacgtctcttcaggccactgactagcgataactttccccacaacggaacaactctcattgcatgggatcattgggtactgtgggtttagtggttgtaaaaacacctgaccgctatccctgatcagtttcttgaaggtaaactcatcacccccaagtctggctatgcagaaatcacctggctcaacagcctgctcagggtcaacgagaattaacattccgtcaggaaagcttggcttggagcctgttggtgcggtcatggaattaccttcaacctcaagccagaatgcagaatcactggcttttttggttgtgcttacccatctctccgcatcacctttggtaaaggttctaagctcaggtgagaacatccctgcctgaacatgagaaaaaacagggtactcatactcacttctaagtgacggctgcatactaaccgcttcatacatctcgtagatttctctggcgattgaagggctaaattcttcaacgctaactttgagaatttttgcaagcaatgcggcgttataagcatttaatgcattgatgccattaaataaagcaccaacgcctgactgccccatccccatcttgtctgcgacagacggctgggataagccaagttcatttttctttttttcataaattgctttaaggcgacgtgcgtcctcaagctgctcttgtgttaatggtttcttttttgtgctcattatatgacctagcgagctggtctacttaagtgctagcagtatctctatcactgatagggatgtcaagcgcgcttgaagagataaattgcactgaaatctagatgtctccagaccgctagtagacgagtccatgtctcgagagcgaggctcttcaagctcgcaaaattgctcttcaagctcgcaaaaaaccccgcccctgacagggcggggttttttcgcgttcgatcccgcgaaatttaattaaccaacaccgccagagatacattaggcaccggcggcttgacactttatgcttccggctcggatactgtgtggatttgacagcggcaggtacgatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagactagtgaattaattcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcagatctaaataataaaaaagccggattaataatctggctttttatattctctctctagtatataaacgcagaaaggcccacccgaaggtgagccagtgtgacgaacccccgcctcagagtgggaaagtctagaaatattttatctgattaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctctccgcagtcgaacgaccgagcgtagcgagtcagtgcgaacgatcaaaaataagtgccttcccatcaaaaaaatattgacaacataaaaaactttgtgttatacttgtaacgctacatggagattaactcaatctagctagagaggctttacactttatgcttccggctcgtataatgtgtggaattgtgagcggataacaatttcacacaggaaacagctatgaccatgattacggattcactggaactctagaccaaagagaggacacaatgcagggttctgtgacagagtttctaaaaccgcgcctggttgatatcgagcaagtgagttcgacgcacgccaaggtgacccttgagcctttagagcgtggctttggccatactctgggtaacgcactgcgccgtattctgctctcatcgatgccgggttgcgcggtgaccgaggttgagattgatggtgtactacatgagtacagcaccaaagaaggcgttcaggaagatatcctggaaatcctgctcaacctgaaagggctggcggtgagagttcagggcaaagatgaagttattcttaccttgaataaatctggcattggccctgtgactgcagccgatatcacccacgacggtgatgtcgaaatcgtcaagccgcagcacgtgatctgccacctgaccgatgagaacgcgtctattagcatgcgtatcaaagttcagcgcggtcgtggttatgtgccggcttctacccgaattcattcggaagaagatgagcgcccaatcggccgtctgctggtcgacgcatgctacagccctgtggagcgtattgcctacaatgttgaagcagcgcgtgtagaacagcgtaccgacctggacaagctggtcatcgaaatggaaaccaacggcacaatcgatcctgaagaggcgattcgtcgtgcggcaaccattctggctgaacaactggaagctttcgttgacttacgtgatgtacgtcagcctgaagtgaaagaagagaaaccagaggcggccgcagcaagcaccgaacgcaaatgggcagaactggcacgtcgtattcgtggtgcaggcggtgttaccctcaatggttttggttgaggatcctctacgccggacgcactcggttccaaaaaaaaaaaaaaagacgctgaaaagcgtcttttttttttttggtccccactggagcacctcaaaaacaccatcatacactaaatcagtaagtt

SEQ ID NO: 55 - DNA, 6782 bp, artificial sequence
  plasmid VN419
gggacaccaggatttatttattctgcgaagtgatcttccgtcacaggtatttattcggcgcaaagtgcgtcgggtgaatgctgccaacttactgattagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagactttcccactctgaggcgggggttcgtcacactggctcaccttcgggtgggcctttctgcgtttatatactagagagagaatataaaaagccagattattaatccggcttttttattatttagatctgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgaattaattcactagtttacttatacagttcatccatacccaaagtgatgcctgcagcagtcacaaattccaggaggaccatgtgatcgcgcttttcgttgggatctttgcttaacgcagattgggtgctaaggtaatgattatccggcagcaagactgggccatcgccaatcggggtattctgctgatagtggtcggctaactgcacactaccatcttcaatgttgtggcgaatcttgaaattcactttgatcccattcttctgtttgtccgccatgatatacacgttatggctgttgtagttgtactccagtttatgacccagaatgttgccatcttctttaaagtcaatgcctttcagctcaatccgattcacaagggtgtcaccctcaaatttgacctctgcacgggttttgtagttcccgtcatctttgaagaagatggtgcgttcttgtacgtaaccttcaggcatcgcggatttaaagaagtcatgctgtttcatgtggtccggataacgcgaaaagcattgaacgccataggtcagcgtagtaaccagtgttggccacggaacaggcagtttgcctgtcgtacagatgaacttcagcgtcagtttgccataggtggcatctccttcaccttcacctgaaaccgaaaatttatgcccattcacatctccatcgagttccactaaaatcggtactacacccgtaaacaactcttcgcctttactgaccatttaactaaccgtgatctttaatactattaactttcacgcgtttattacgacatcccggtgtgtagccgttcgaccacgctgccgagcctgagatgctgctcgtactcctgaagatccccgaagtcgatcgtgcgagtcagcccgccgcggacgtcgaacgtcagccgaacgttcatcgaccgaagccaggtgttctttgccgcggtgtcctgctcccgccaccagtccccgaaccgctgcccggtctcgcgccactcccagccagacgggcgagcctccaggccctccagctcctcttgccgcgcggccagcgccgcaatacgggcatccagtgcttctcgctgcggagagcccgcccggtaggccggggagccgatcagcgacgtcaggtccaccagctccgcgttcacctccgcgagttcgaccgcggagtccgagcccgctacccagactttctccagacgctccgcgtccccgagcagatccagtacctgctcctcgcagaacgcgtcccactcggccatcgccaccgtgccgttcccgcagtgcttcgggaaccccatcgagcggcagcggtagcgcgggtgcttacgtcctcccccggcgaacttgtacgcgggctccccgcacaccgcgcagaacaacacccgcagcagcagcgacggggtagacaccgcgggcttcgcccgggaggtcttcacgagctcggcgcgcagcgcctccagctgctcacgggtcaggatcggctcagcccgcaccagcggggctccgtcgtcgtctcggacggtcttaccgttcagagtcgcgtacccgagcatcgccccggagatcatcgatcgcttcagcgcggtagccgaccactcccggccctgcggctcgcggccttgcagctgcgcgaagtagtccttcggcgacaggacaccacgccggttcaggtcgtgggccaccagatgcagcggctcgtggttgtcgacgacgcggtgatacacctcgaggatgcgctctcgctgcacagggtccggcaccagccgccactccccgtccacgcgcgtaggcaggtatccccacggcggcagggatcctcggtatttcccggcgcggatattgaaatgcgcagccgaacggttccgctctttgatcgcttctaattccatctgcgccaccgttcccataagcgcgatgacgaccgccgcaaacggcgtcgtcgtatcgaagtgcgcttcggtcgcggagacgaccagcttcttgtggtcctcggcccagtggaccagctgctgaagatgccggatcgatcgggtcaaccgatctacccggtacgccacgatcacgtcaaacggttgctcctcgaacgctagccaccgggccaggttcggtctgcgcttccggtcgaacggatcgaccgccccggagacgtccagatcctccgctaccccgacgacgtcccagccgcgctgggcgcagagctgctggcaagactccagctgacgctccggtgaagtcgtagcatcggtgacgcgggacaggcggatgactaccagggctctcatggtaccdktagttatmckcccktctaaaaatactgtggagtcgtacgtctctatcactgatagggaacgtgctctctatcactgatagggacaattacgttaaatctatcaccacaagggataaatatctagcctggggtgcctaatgtaacaccgtgcgtgttgactattttacctctggcggtgataggttaattaaatttcgcgggatcgaacgcgaaaaaaccccgccctgtcaggggcggggttttttgcgagcttgaagagcctcgctctcgagtctggtaaagaaaccgctgctgcgaaatttgaacgccagcacatggactcgtctactagcggtctggagacattctagatttcagtgcaatttatctcttcaaatgtagcacctgaagtcagccccatacgatataagttgtaattctcatgtttgacagcttatcatcgataagctaattctcactcattaggcaccccaggctttacactttatgcttccggctcgtataatgtgtggaattgtgagcggataacaatttcacacaggaaacagcgtatgagcacaaaaaagaaaccattaacacaagagcagcttgaggacgcacgtcgccttaaagcaatttatgaaaaaaagaaaaatgaacttggcttatcccaggaatctgtcgcagacaagatggggatggggcagtcaggcgttggtgctttatttaatggcatcaatgcattaaatgcttataacgccgcattgcttgcaaaaattctcaaagttagcgttgaagaatttagcccttcaatcgccagagaaatctacgagatgtatgaagcggttagtatgcagccgtcacttagaagtgagtatgagtaccctgttttttctcatgttcaggcagggatgttctcacctgagcttagaacctttaccaaaggtgatgcggagagatgggtaagcacaaccaaaaaagccagtgattctgcattctggcttgaggttgaaggtaattccatgaccgcaccaacaggctccaagccaagctttcctgacggaatgttaattctcgttgaccctgagcaggctgttgagccaggtgatttctgcatagccagacttgggggtgatgagtttaccttcaagaaactgatcagggatagcggtcaggtgtttttacaaccactaaacccacagtacccaatgatcccatgcaatgagagttgttccgttgtggggaaagttatcgctagtcagtggcctgaagagacgtttggcgcggccgcacttggtgaagaggacattccgcgtgaacctcgtcgcattgtgattcatcgcggttctacgggcttaggctttaacatcgttggtggcgaagatggtgaagggatcttcatctccttcattctggctggtggaccagctgatctgtcaggggaactccgtaaaggcgatcagattctgagcgtcaatggcgtagacttgcggaatgccagtcacgaacaagcggcaattgcgctgaagaacgcaggacagactgtgaccatcatcgcgcagtacaaaccggaagagtattcgcgctttgaggccaaatgaggatccttaccttacagatctgcatcgcaggatgctgctggctaccctgtggaacacctacatctgtattaacgaagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaaca

SEQ ID NO: 56 - DNA, 7879 bp, artificial sequence
  plasmid VN376
gcaataaaaaatcaaatcggatttcactatataatctcactttatctaagatgaatccgatggaagcatcctgttttctctcaatttttttatctaaaacccagcgttcgatgcttctttgagcgaacgatcaaaaataagtgccttctctagattttcagcaggacgcactgacctccctatcagtgatagagattgacatccctatcagtgatagagatactgagcacatatattaaagaggagaaagccatggcgcagggttctgtgacagagtttctaaaaccgcgcctggttgatatcgagcaagtgagttcgacgcacgccaaggtgacccttgagcctttagagcgtggctttggccatactctgggtaacgcactgcgccgtattctgctctcatcgatgccgggttgcgcggtgaccgaggttgagattgatggtgtactacatgagtacagcaccaaagaaggcgttcaggaagatatcctggaaatcctgctcaacctgaaagggctggcggtgagagttcagggcaaagatgaagttattcttaccttgaataaatctggcattggccctgtgactgcagccgatatcacccacgacggtgatgtcgaaatcgtcaagccgcagcacgtgatctgccacctgaccgatgagaacgcgtctattagcatgcgtatcaaagttcagcgcggtcgtggttatgtgccggcttctacccgaattcattcggaagaagatgagcgcccaatcggccgtctgctggtcgacgcatgctacagccctgtggagcgtattgcctacaatgttgaagcagcgcgtgtagaacagcgtaccgacctggacaagctggtcatcgaaatggaaaccaacggcacaatcgatcctgaagaggcgattcgtcgtgcggcaaccattctggctgaacaactggaagctttcgttgacttacgtgatgtacgtcagcctgaagtgaaagaagagaaaccagaggcggccgcataatgaaactacaaacagaccagcgtttgaggatcctctactcacactggctcaccttcgggtgggcctttctgcgtttatatactagagagagaatataaaaagccagattattaatccggcttttttattatttagttacgctagggataacagggtaatatagggtacctttacggctagctcagtcctaggtactatgctagctactattaattaatcggccggcttgtcgacgacggcggtctccgtcgtcaggatcatccgggcccatgcgcacccttagcgagaggtttatcattaaggtcaacctctggatgttgtttcggcatcctgcattgaatctgagttactgtctgttttcctagtagaggatcctcaaacgctggtctgtttgtagtttttggttgcggccgcctctggtttctcttctttcactaggaaacccgttttttctgacgtaagggtgcgcaactttcatgaaatccgctgaatatttgaacacttttagattgagaaatctcggcctacctgtcatgaacaatttgcatgacatgtctaaggcgactcgcatatctgttgaaacacttcggttgttaatctatacagctgattttcgctataggatctacactgtagaaaagaaaggcccagagaagagaatgagaaccatttaccaaccttctcgagaacttaaagccttacaaggatgggttctacgtaacattttagataaactgtcgtcatctcctttttctattggatttgaaaagcaccaatctattttgaataatgctaccccgcatattggggcaaactttatactgaatattgatttggaggattttttcccaagtttaactgctaacaaagtttttggagtgttccattctcttggttataatcgactaatatcttcagttttgacaaaaatatgttgttataaaaatctgctaccacaaggtgctccatcatcacctaaattagctaatctaatatgttctaaacttgattatcgtattcagggttatgcaggtagtcggggcttgatatatacgagatatgccgatgatctcaccttatctgcacagtctatgaaaaaggttgttaaagcacgtgattttttattttctataatcccaagtgaaggattggttattaactcaaaaaaaacttgtattagtgggcctcgtagtcagaggaaagttacaggtttagttatttcacaagagaaagttgggataggtagagaaaaatataaagaaattagagcaaagatacatcatatattttgcggtaagtcttctgagatagaacacgttaggggatggttgtcatttattttaagtgtggattcaaaaagccataggagattaataacttatattagcaaattagaaaaaaaatatggaaagaaccctttaaataaagcgaagacctaaagatctctaacaattcattaaagaggagaaaggtactatgagtactgcactcgcaacgctggctgggaagctggctgaacgtgtcggcatggattctgtcgacccacaggaactgatcaccactcttcgccagacggcatttaaaggtgatgccagcgatgcgcagttcatcgcattactgatcgttgccaaccagtacggccttaatccgtggacgaaagaaatttacgcctttcctgataagcagaatggcatcgttccggtggtgggcgttgatggctggtcccgcatcatcaatgaaaaccagcagtttgatggcatggactttgagcaggacaatgaatcctgtacatgccggatttaccgcaaggaccgtaatcatccgatctgcgttaccgaatggatggatgaatgccgccgcgaaccattcaaaactcgcgaaggcagagaaatcacggggccgtggcagtcgcatcccaaacggatgttacgtcataaagccatgattcagtgtgcccgtctggccttcggatttgctggtatctatgacaaggatgaagccgagcgcattgtcgaaaatactgcatacactgcagaacgtcagccggaacgcgacatcactccggttaacgatgaaaccatgcaggagattaacactctgctgatcgccctggataaaacatgggatgacgacttattgccgctctgttcccagatatttcgccgcgacattcgtgcatcgtcagaactgacacaggccgaagcagtaaaagctcttggattcctgaaacagaaagccgcagagcagaaggtggcagcatgaggtaatgaactagtttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatggtatatctccttcttaaagttcgtacggtcggggtttgtaccgtacaccactgagaccgcggtggttgaccagacaaaccacgaccaattgtataaacgcagaaaggcccacccgaaggtgagccagtgtgactctagtagagagcgttcaccgacaaacaacagataaaacgaaaggcccagtctttcgactgagcctttcgttttatttgatgcctggctcgagagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtcgagctcttaggacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctggacatcattaattcctaattttatgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgt

SEQ ID NO: 57 - protein, 441 aa, artificial sequence
  HIV-1 RT p51
MPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKQKKSVTVLDVGDAYFSVPLDKDFRKYTAFTIPSINNETPGIRYQYNVLPQGWKGSPAIFQCSMTKILEPFRKQNPDIVIYQYMDDLYVGSDLEIGQHRTKIEELRQHLLRWGFTTPDKKHQKEPPFLWMGYELHPDKWTVQPIVLPEKDSWTVNDIQKLVGKLNWASQIYAGIKVRQLCKLLRGTKALTEVVPLTEEAELELAENREILKEPVHGVYYDPSKDLIAEIQKQGQGQWTYQIYQEPFKNLKTGKYARMKGAHTNDVKQLTEAVQKIATESIVIWGKTPKFKLPIQKETWEAWWTEYWQATWIPEWEFVNTPPLVKLWYQLEKEPIIGAETF

SEQ ID NO: 58 - DNA, 7879 bp, artificial sequence
  plasmid VN405
gcaataaaaaatcaaatcggatttcactatataatctcactttatctaagatgaatccgatggaagcatcctgttttctctcaatttttttatctaaaacccagcgttcgatgcttctttgagcgaacgatcaaaaataagtgccttctctagattttcagcaggacgcactgacctccctatcagtgatagagattgacatccctatcagtgatagagatactgagcacatatattaaagaggagaaagccatggcgcagggttctgtgacagagtttctaaaaccgcgcctggttgatatcgagcaagtgagttcgacgcacgccaaggtgacccttgagcctttagagcgtggctttggccatactctgggtaacgcactgcgccgtattctgctctcatcgatgccgggttgcgcggtgaccgaggttgagattgatggtgtactacatgagtacagcaccaaagaaggcgttcaggaagatatcctggaaatcctgctcaacctgaaagggctggcggtgagagttcagggcaaagatgaagttattcttaccttgaataaatctggcattggccctgtgactgcagccgatatcacccacgacggtgatgtcgaaatcgtcaagccgcagcacgtgatctgccacctgaccgatgagaacgcgtctattagcatgcgtatcaaagttcagcgcggtcgtggttatgtgccggcttctacccgaattcattcggaagaagatgagcgcccaatcggccgtctgctggtcgacgcatgctacagccctgtggagcgtattgcctacaatgttgaagcagcgcgtgtagaacagcgtaccgacctggacaagctggtcatcgaaatggaaaccaacggcacaatcgatcctgaagaggcgattcgtcgtgcggcaaccattctggctgaacaactggaagctttcgttgacttacgtgatgtacgtcagcctgaagtgaaagaagagaaaccagaggcggccgcaaccaaaaactacaaacagaccagcgtttgaggatcctctactcacactggctcaccttcgggtgggcctttctgcgtttatatactagagagagaatataaaaagccagattattaatccggcttttttattatttagttacgctagggataacagggtaatatagggtacctttacggctagctcagtcctaggtactatgctagctactattaattaatcggccggcttgtcgacgacggcggtctccgtcgtcaggatcatccgggcccatgcgcacccttagcgagaggtttatcattaaggtcaacctctggatgttgtttcggcatcctgcattgaatctgagttactgtctgttttcctagtagaggatcctcaaacgctggtctgtttgtagtttttggttgcggccgcctctggtttctcttctttcactaggaaacccgttttttctgacgtaagggtgcgcaactttcatgaaatccgctgaatatttgaacacttttagattgagaaatctcggcctacctgtcatgaacaatttgcatgacatgtctaaggcgactcgcatatctgttgaaacacttcggttgttaatctatacagctgattttcgctataggatctacactgtagaaaagaaaggcccagagaagagaatgagaaccatttaccaaccttctcgagaacttaaagccttacaaggatgggttctacgtaacattttagataaactgtcgtcatctcctttttctattggatttgaaaagcaccaatctattttgaataatgctaccccgcatattggggcaaactttatactgaatattgatttggaggattttttcccaagtttaactgctaacaaagtttttggagtgttccattctcttggttataatcgactaatatcttcagttttgacaaaaatatgttgttataaaaatctgctaccacaaggtgctccatcatcacctaaattagctaatctaatatgttctaaacttgattatcgtattcagggttatgcaggtagtcggggcttgatatatacgagatatgccgatgatctcaccttatctgcacagtctatgaaaaaggttgttaaagcacgtgattttttattttctataatcccaagtgaaggattggttattaactcaaaaaaaacttgtattagtgggcctcgtagtcagaggaaagttacaggtttagttatttcacaagagaaagttgggataggtagagaaaaatataaagaaattagagcaaagatacatcatatattttgcggtaagtcttctgagatagaacacgttaggggatggttgtcatttattttaagtgtggattcaaaaagccataggagattaataacttatattagcaaattagaaaaaaaatatggaaagaaccctttaaataaagcgaagacctaaagatctctaacaattcattaaagaggagaaaggtactatgagtactgcactcgcaacgctggctgggaagctggctgaacgtgtcggcatggattctgtcgacccacaggaactgatcaccactcttcgccagacggcatttaaaggtgatgccagcgatgcgcagttcatcgcattactgatcgttgccaaccagtacggccttaatccgtggacgaaagaaatttacgcctttcctgataagcagaatggcatcgttccggtggtgggcgttgatggctggtcccgcatcatcaatgaaaaccagcagtttgatggcatggactttgagcaggacaatgaatcctgtacatgccggatttaccgcaaggaccgtaatcatccgatctgcgttaccgaatggatggatgaatgccgccgcgaaccattcaaaactcgcgaaggcagagaaatcacggggccgtggcagtcgcatcccaaacggatgttacgtcataaagccatgattcagtgtgcccgtctggccttcggatttgctggtatctatgacaaggatgaagccgagcgcattgtcgaaaatactgcatacactgcagaacgtcagccggaacgcgacatcactccggttaacgatgaaaccatgcaggagattaacactctgctgatcgccctggataaaacatgggatgacgacttattgccgctctgttcccagatatttcgccgcgacattcgtgcatcgtcagaactgacacaggccgaagcagtaaaagctcttggattcctgaaacagaaagccgcagagcagaaggtggcagcatgaggtaatgaactagtttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatggtatatctccttcttaaagttcgtacggtcggggtttgtaccgtacaccactgagaccgcggtggttgaccagacaaaccacgaccaattgtataaacgcagaaaggcccacccgaaggtgagccagtgtgactctagtagagagcgttcaccgacaaacaacagataaaacgaaaggcccagtctttcgactgagcctttcgttttatttgatgcctggctcgagagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtcgagctcttaggacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctggacatcattaattcctaattttatgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgt

SEQ ID NO: 59 - DNA, 8838 bp, artificial sequence
  plasmid VN1238
ggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggtgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccagagcctgataaaaacggttagcgcttcgttaatacagatgtaggtgttccacagggtagccagcagcatcctgcgatgcagatctgtaaggtaagagctcttaaatatgagcgtcacctttagttgctttgccatttacagtaacctgaccttgctcattaactgtaaaggtaatagcagttgctacctcataaccgtctggtgctgcggtttcgacaaatgtatattttcctggatacaggtagaaatctttcacttgtccatctgaaatccatgtactaatagttttaccagatgaatcacgcaactccatagttgcaccagctaactctttgccgtcctcatcacgttttgagaatttaatatgggtagcactatcttcttcaattgtcatatcaccggactgaccttgctcacttgataaacctgataaggtatcaaccatcgcgcctgcggtaccgccaaacgtctcttcaggccactgactagcgataactttccccacaacggaacaactctcattgcatgggatcattgggtactgtgggtttagtggttgtaaaaacacctgaccgctatccctgatcagtttcttgaaggtaaactcatcacccccaagtctggctatgcagaaatcacctggctcaacagcctgctcagggtcaacgagaattaacattccgtcaggaaagcttggcttggagcctgttggtgcggtcatggaattaccttcaacctcaagccagaatgcagaatcactggcttttttggttgtgcttacccatctctccgcatcacctttggtaaaggttctaagctcaggtgagaacatccctgcctgaacatgagaaaaaacagggtactcatactcacttctaagtgacggctgcatactaaccgcttcatacatctcgtagatttctctggcgattgaagggctaaattcttcaacgctaactttgagaatttttgcaagcaatgcggcgttataagcatttaatgcattgatgccattaaataaagcaccaacgcctgactgccccatccccatcttgtctgcgacagacggctgggataagccaagttcatttttctttttttcataaattgctttaaggcgacgtgcgtcctcaagctgctcttgtgttaatggtttcttttttgtgctcatacgctgtttcctgtgtgaaattgttatccgctcacaattccacacattatacgagccggaagcataaagtgtaaagcctggggtgcctaatgagtgagaattagcttatcgatgataagctgtcaaacatgagaattacaacttatatcgtatggggctgacttcaggtgctacatttgaagagataaattgcactgaaatctagatgtctccagaccgctagtagacgagtccatgtgctggcgttcaaatttcgcagcagcggtttctttaccagactcgagagcgaggctcttcaagctcgcaaaattgctcttcaagctcgcaaaaaaccccgcccctgacagggcggggttttttcgcgttcgatcccgcgaaatttaattaaccaacaccgccagagatacattaggcaccccaggcttgacactttatgcttccggctcggatactgtgtggaattgaaagaggaacgtacgatggtcagtaaaggcgaagagttgtttacgggtgtagtaccgattttagtggaactcgatggagatgtgaatgggcataaattttcggtttcaggtgaaggtgaaggagatgccacctatggcaaactgacgctgaagttcatctgtacgacaggcaaactgcctgttccgtggccaacactggttactacgctgacctatggcgttcaatgcttttcgcgttatccggaccacatgaaacagcatgacttctttaaatccgcgatgcctgaaggttacgtacaagaacgcaccatcttcttcaaagatgacgggaactacaaaacccgtgcagaggtcaaatttgagggtgacacccttgtgaatcggattgagctgaaaggcattgactttaaagaagatggcaacattctgggtcataaactggagtacaactacaacagccataacgtgtatatcatggcggacaaacagaagaatgggatcaaagtgaatttcaagattcgccacaacattgaagatggtagtgtgcagttagccgaccactatcagcagaataccccgattggcgatggcccagtcttgctgccggataatcattaccttagcacccaatctgcgttaagcaaagatcccaacgaaaagcgcgatcacatggtcctcctggaatttgtgactgctgcaggcatcactttgggtatggatgaactgtataagtgaaattcctggtccaggaatttcacatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagggcccactccacagtatttttagamgggmgkataactamhatgagagccctggtagtcatccgcctgtcccgcgtcaccgatgctacgacttcaccggagcgtcagctggagtcttgccagcagctctgcgcccagcgcggctgggacgtcgtcggggtagcggaggatctggacgtctccggggcggtcgatccgttcgaccggaagcgcagaccgaacctggcccggtggctagcgttcgaggagcaaccgtttgacgtgatcgtggcgtaccgggtagatcggttgacccgatcgatccggcatcttcagcagctggtccactgggccgaggaccacaagaagctggtcgtctccgcgaccgaagcgcacttcgatacgacgacgccgtttgcggcggtcgtcatcgcgcttatgggaacggtggcgcagatggaattagaagcgatcaaagagcggaaccgttcggctgcgcatttcaatatccgcgccgggaaataccgaggaagcctgccgccgtggggatacctgcctacgcgcgtggacggggagtggcggctggtgccggaccctgtgcagcgagagcgcatcctcgaggtgtatcaccgcgtcgtcgacaaccacgagccgctgcatctggtggcccacgacctgaaccggcgtggtgtcctgtcgccgaaggactacttcgcgcagctgcaaggccgcgagccgcagggccgggagtggtcggctaccgcgctgaagcgatcgatgatctccggggcgatgctcgggtacgcgactctgaacggtaagaccgtccgagacgacgacggagccccgctggtgcgggctgagccgatcctgacccgtgagcagctggaggcgctgcgcgccgagttggtgaagacctcccgggcgaagcccgcggtgtctaccccgtcgctgctgctgcgggtgttgttctgcgcggtgtgcggggagcccgcgtacaagttcgccgggggaggacgtaagcacccgcgctaccgctgccgctcgatggggttcccgaagcactgcgggaacggcacggtggcgatggccgagtgggacgcgttctgcgaggagcaggtactggatctgctcggggacgcggagcgtctggagaaagtctgggtagcgggctcggactccgcggtcgaactcgcggaggtgaacgcggagctggtggacctgacgtcgctgatcggctccccggcctaccgggcgggctctccgcagcgagaagcactggatgcccgtattgcggcgctggccgcgcggcaagaggagctggagggcctggaggctcgcccgtctggctgggagtggcgcgagaccgggcagcggttcggggactggtggcgggagcaggacaccgcggcaaagaacacctggcttcggtcgatgaacgttcggctgacgttcgacgtccgcggcgggctgactcgcacgatcgacttcggggatcttcaggagtacgagcagcatctcaggctcggcagcgtggtcgaacggctacacaccgggatgtcgtaataaactagtgaattaattcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcagatctaaataataaaaaagccggattaataatctggctttttatattctctctctagtatataaacgcagaaaggcccacccgaaggtgagccagtgtgacgaacccccgcctcagagtgggaaagtctagaaatattttatctgattaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctctccgcagtcgaacgaccgagcgtagcgagtcagtgcgaacgatcaaaaataagtgccttcccatcaaaaaaatattgacaacataaaaaactttgtgttatacttgtaacgctacatggagattaactcaatctagctagagaggctttacactttatgcttccggctcgtataatgtgtggaattgtgagcggataacaatttcacacaggaaacagctatgaccatgattacggattcactggaactctagaccaaagagaggacacaatgcagggttctgtgacagagtttctaaaaccgcgcctggttgatatcgagcaagtgagttcgacgcacgccaaggtgacccttgagcctttagagcgtggctttggccatactctgggtaacgcactgcgccgtattctgctctcatcgatgccgggttgcgcggtgaccgaggttgagattgatggtgtactacatgagtacagcaccaaagaaggcgttcaggaagatatcctggaaatcctgctcaacctgaaagggctggcggtgagagttcagggcaaagatgaagttattcttaccttgaataaatctggcattggccctgtgactgcagccgatatcacccacgacggtgatgtcgaaatcgtcaagccgcagcacgtgatctgccacctgaccgatgagaacgcgtctattagcatgcgtatcaaagttcagcgcggtcgtggttatgtgccggcttctacccgaattcattcggaagaagatgagcgcccaatcggccgtctgctggtcgacgcatgctacagccctgtggagcgtattgcctacaatgttgaagcagcgcgtgtagaacagcgtaccgacctggacaagctggtcatcgaaatggaaaccaacggcacaatcgatcctgaagaggcgattcgtcgtgcggcaaccattctggctgaacaactggaagctttcgttgacttacgtgatgtacgtcagcctgaagtgaaagaagagaaaccagaggcggccgcagccaagtgaccagtgcagttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggacgcggcacacatcgtgatggtggccgcctacaagccgacgaagtaaggatcctctacgccggacgcactcggttccaaaaaaaaaaaaaaagacgctgaaaagcgtcttttttttttttggtccccactggagcacctcaaaaacaccatcatacactaaatcagtaagtt

SEQ ID NO: 60 - DNA, 8087 bp, artificial sequence
  plasmid VN1228
gcaataaaaaatcaaatcggatttcactatataatctcactttatctaagatgaatccgatggaagcatcctgttttctctcaatttttttatctaaaacccagcgttcgatgcttctttgagcgaacgatcaaaaataagtgccttctctagattttcagcaggacgcactgacctccctatcagtgatagagattgacatccctatcagtgatagagatactgagcacatatattcggccggcttgtcgacgacggcggtctccgtcgtcaggatcatccgggcaagcttaacgatcgaataaggaggtacgaacatggatgctcagacgcgacgccgggaacgccgcgcggaaaaacaagctcagtggaaggcggcaaatgggggcggaggttcggggggcggtggttcaatctcatcaagcaagcatacattatctcaaatgaataaagtctcaaacattgttaaggaaccagagttgccggatatctataaagagttcaaagacatcaccgcagacaccaacacggagaagctcccaaaacccataaagggccttgagtttgaagttgaactgacgcaagaaaactaccgattaccaatacgaaactatcccttacctccgggtaaaatgcaagctatgaatgacgaaataaaccaaggcctgaaatcaggcatcatccgagagtccaaagcaattaatgcgtgcccagtaatgttcgtaccaaaaaaagagggcacgttgcgaatggttgttgactacaagccacttaataaatatgtaaagcctaatatctacccattaccactaatcgaacaacttcttgctaagattcaaggctccaccatctttacaaaactggatttaaaatcagcgtaccatttaattcgagtgcgtaaaggagacgaacataagctggccttccgttgcccgcgaggggtctttgaatacttggtcatgccgtacggtatttcaacggccccggcgcatttccagtactttatcaatacgatccttggtgaagctaaagagagtcacgttgtatgctatatggacgatatcttgattcatagcaaatcggaatcggaacatgttaaacatgttaaagacgttctgcaaaagctgaaaaacgcgaacctgatcataaatcaagctaaatgcgaatttcaccaaagccaggtgaaatttataggctatcatatctccgaaaagggcttcacaccgtgccaggaaaacattgataaggtgcttcagtggaagcaaccaaagaaccgtaaagagttgagacagtttttggggtctgtcaactacttgcggaaatttataccaaaaacgtcacagcttactcatccacttaacaaactactcaaaaaagatgtgcgttggaaatggacgccgacccaaacgcaggcgatcgagaatataaaacagtgtttggtaagccccccggttctacgccacttcgacttctcaaaaaagatactgctcgaaactgatgcatcagatgtagccgttggagcggttctgtcccagaaacatgacgatgataaatattatccagtggggtattattcggcaaagatgagtaaagcccaattaaattattcagtctcggataaagagatgctggctattatcaagtcgctgaaacattggcggcactacttagaaagtaccattgagccctttaagatcctcacggatcatcgtaatcttataggtcgaataactaatgaatctgaaccagaaaacaaacggttagcccgttggcagctatttctacaggactttaattttgaaatcaattaccgtcccggttccgcgaatcatatcgctgacgccctctcacgaatagtagatgaaacagagccgattccaaaagactctgaggataattcaatcaacttcgtgaaccaaatctcaatttgataaagatctatacaggtcaaaatactacggttacatataatgagtactgcactcgcaacgctggctgggaagctggctgaacgtgtcggcatggattctgtcgacccacaggaactgatcaccactcttcgccagacggcatttaaaggtgatgccagcgatgcgcagttcatcgcattactgatcgttgccaaccagtacggccttaatccgtggacgaaagaaatttacgcctttcctgataagcagaatggcatcgttccggtggtgggcgttgatggctggtcccgcatcatcaatgaaaaccagcagtttgatggcatggactttgagcaggacaatgaatcctgtacatgccggatttaccgcaaggaccgtaatcatccgatctgcgttaccgaatggatggatgaatgccgccgcgaaccattcaaaactcgcgaaggcagagaaatcacggggccgtggcagtcgcatcccaaacggatgttacgtcataaagccatgattcagtgtgcccgtctggccttcggatttgctggtatctatgacaaggatgaagccgagcgcattgtcgaaaatactgcatacactgcagaacgtcagccggaacgcgacatcactccggttaacgatgaaaccatgcaggagattaacactctgctgatcgccctggataaaacatgggatgacgacttattgccgctctgttcccagatatttcgccgcgacattcgtgcatcgtcagaactgacacaggccgaagcagtaaaagctcttggattcctgaaacagaaagccgcagagcagaaggtggcagcatgaggtaatgaacgcgtttcagccaaaaaacttaagaccgccggtcttgtccactaccttgcagtaatgcggtggacaggatcggcggttttcttttctcttctcaacgtacgttatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattatgaattcgtcggggtttgtaccgtacaccactgagaccgcggtggttgaccagacaaaccacgaccaaaacgaaaaaagacgcttttcagcgtcttttttcgaacggccgctgcaggcttcctcgctcattaattaacgatcccgcgaaatttgacatccctatcagtgatagatataatgctagcagcctgaagtgaaagaagagaaaccagaggcggccgcagccaagttgaccagtgcagttccggtgctcaccgcgcgcgacgtcgccggagactagtaatgagagtaaagcactagatattggtgtaaaatcacccgccagcagattatacctgctggttttttttatactttcaccagcgtttctgggtgagcaaaaacaggaaggcaaaggcgcgccatgccgcaaaaaagggaataagggcgacacggaaatgttgaataccagaaaagaggcctcccgaaaggggggccttttttcgttttggtcctcatctccagagacaatgccaagaacacggtgtatctgcaaatgaacagcctaaaacctgaggacacggccgtttattactgtgcgacagatcgtccatggaggcattttgctattaagggattgacgtccctatcagtgatagataagatgcgccccgcattgggccctgaagaagggcccgggccctgaagaagggcccaacaacaacaacaacaacaactctagtgctttactctcattcggtgattggcgcagcctggtagcgcacttcgttcgggacgaaggggtcggaggttcgaatcctctatcaccgaccaaattcgaaaagcctgctcaacgagcaggcttttttgcatctcgagagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtcgagctcttaggacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctggacatcattaattcctaattttatgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgt

SEQ ID NO: 61 - protein, 421 aa, artificial sequence
  rhlB (Escherichia coli)
MSKTHLTEQKFSDFALHPKVVEALEKKGFHNCTPIQALALPLTLAGRDVAGQAQTGTGKTMAFLTSTFHYLLSHPAIADRKVNQPRALIMAPTRELAVQIHADAEPLAEATGLKLGLAYGGDGYDKQLKVLESGVDILIGTTGRLIDYAKQNHINLGAIQVVVLDEADRMYDLGFIKDIRWLFRRMPPANQRLNMLFSATLSYRVRELAFEQMNNAEYIEVEPEQKTGHRIKEELFYPSNEEKMRLLQTLIEEEWPDRAIIFANTKHRCEEIWGHLAADGHRVGLLTGDVAQKKRLRILDEFTRGDLDILVATDVAARGLHIPAVTHVFNYDLPDDCEDYVHRIGRTGRAGASGHSISLACEEYALNLPAIETYIGHSIPVSKYNPDALMTDLPKPLRLTRPRTGNGPRRTGAPRNRRRSG

SEQ ID NO: 62 - protein, 278 aa, artificial sequence
  dam (Escherichia coli)
MKKNRAFLKWAGGKYPLLDDIKRHLPKGECLVEPFVGAGSVFLNTDFSRYILADINSDLISLYNIVKMRTDEYVQAARELFVPETNCAEVYYQFREEFNKSQDPFRRAVLFLYLNRYGYNGLCRYNLRGEFNVPFGRYKKPYFPEAELYHFAEKAQNAFFYCESYADSMARADDASVVYCDPPYAPLSATANFTAYHTNSFTLEQQAHLAEIAEGLVERHIPVLISNHDTMLTREWYQRAKLHVVKVRRSISSNGGTRKKVDELLALYKPGVVSPAKK

SEQ ID NO: 63 - protein, 134 aa, artificial sequence
  rne (Escherichia coli), fragment 711-844
ERVRPVQPRRKQRQLNQKVRYEQSVAEEAVVAPVVEETVAAEPIVQEAPAPRTELVKVPLPVVAQTAPEQQEENNADNRDNGGMPRRSRRSPRHLRVSGQRRRRYRDERYPTQSPMPLTVACASPELASGKVWI

SEQ ID NO: 64 - DNA, 1430 bp, artificial sequence
  xonA (Escherichia coli)
atgatgaatgacggtaagcaacaatcttgacctttttgtttcacgattacgaaacctttggcacgcaccccgcgttagatcgccctgcacagttcgcagccattcgcaccgatagcgaattcaatgtcatcggcgaacccgaagtcttttactgcaagcccgctgatgactatttaccccagccaggagccgtattaattaccggtattaccccgcaggaagcacgggcgaaaggagaaaacgaagccgcgtttgccgcccgtattcactcgctttttaccgtaccgaagacctgtattctgggctacaacaatgtgcgtttcgacgacgaagtcacacgcaacattttttatcgtaatttctacgatccttacgcctggagctggcagcatgataactcgcgctgggatttactggatgttatgcgtgcctgttatgccctgcgcccggaaggaataaactggcctgaaaatgatgacggtctaccgagctttcgccttgagcatttaaccaaagcgaatggtattgaacatagcaacgcccacgatgcgatggctgatgtgtacgccactattgcgatggcaaagctggtaaaaacgcgtcagccacgcctgtttgattatctctttacccatcgtaataaacacaaactgatggcgttgattgatgttccgcagatgaaacccctggtgcacgtttccggaatgtttggagcatggcgcggcaataccagctgggtggcaccgctggcgtggcatcctgaaaatcgcaatgccgtaattatggtggatttggcaggagacatttcgccattactggaactggatagcgacacattgcgcgagcgtttatataccgcaaaaaccgatcttggcgataacgccgccgttccggttaagctggtgcatatcaataaatgtccggtgctggcccaggcgaatacgctacgcccggaagatgccgaccgactgggaattaatcgtcagcattgcctcgataacctgaaaattctgcgtgaaaatccgcaagtgcgcgaaaaagtggtggcgatattcgcggaagccgaaccgtttacgccttcagataacgtggatgcacagctttataacggctttttcagtgacgcagatcgtgcagcaatgaaaattgtgctggaaaccgagccgcgtaatttaccggcactggatatcacttttgttgataaacggattgaaaagctgttgttcaattatcgggcacgcaacttcccggggacgctggattatgccgagcagcaacgctggctggagcaccgtcgccaggtcttcacgccagagtttttgcagggttatgctgatgaattgcagatgctggtacaacaatatgccgatgacaaagagaaagtggcgctgttaaaagcactttggcagtacgcggaagagattgtctaa

SEQ ID NO: 65 - DNA, 665 bp, artificial sequence
  exoX (Escherichia coli)
atgttgcgcattatcgatacagaaactgatgcggtttgcagggagggatcgttgagattgcctctgttgatgtcattgacggaaaaatcgtcaaccccatgagccacctggtgcgccccgatcgtcctattagtccacaagcgatggcgattcatcgcatcaccgaagccatggtcgccgataaaccgtggattgaagatgtgatcccacactattacggtagtgaatggtatgtcgcgcataacgccagctttgaccgccgcgtactgcctgagatgcccggtgagtggatttgcactatgaaactggcccgtcgtttgtggcctgggatcaagtacagcaatatggcgttatataaaacacgcaagctcaatgtacagacgccgccgggcctgcatcatcaccgcgcgttgtatgactgttatatcaccgccgcgttgcttatcgatattatgaacacctccggctggacggcagaacagatggccgatatcaccggacgtccgtcgttgatgacgaccttcacctttggcaaataccgtggcaaagcggtttccgacgttgccgaacgcgatccgggctatctgcgctggttatttaataacctggacagcatgagcccggagctgcgtttaacactgaaacattatctggaaaatacttag

SEQ ID NO: 66 - DNA, 1373 bp, artificial sequence
  xseA (Escherichia coli)
atgttaccttctcaatcccctgcaattgatttaccgttagtcgcctgaatcaaacggttcgtctgctgcttgagcatgagatgggacaggtttggatcagcggcgaaatttctaatttcacgcaaccagcttccggtcactggtactttacactcaaagacgacaccgcccaggtacgctgcgcgatgttccgcaacagcaaccgccgggtgaccttccgcccacagcatgggcaacaagttttagttcgcgccaatattacgctctacgagccgcgcggcgactaccagataatcgttgagagtatgcagccggccggtgaagggctgctgcaacagaagtacgaacagctcaaagcgaagttgcaggctgaaggtttgttcgatcagcaatacaaaaaaccacttccctcccctgcgcattgcgttggtgtgatcacctcaaaaaccggtgctgcgctacatgatattttgcatgtgttaaaacgtcgcgatccttctctgccggtgatcatctaccctgccgccgttcagggcgatgacgcgccggggcaaattgttcgcgccattgaactggcgaatcagcgcaatgagtgcgacgtattgatcgtcgggcgcggcggcggttcgctggaagatttatggagttttaacgacgaacgcgtagcgcgggcgatttttaccagccgcattccggttgtcagcgccgtcgggcatgagacggatgtgaccattgccgattttgttgccgatctgcgtgcgccaacgccgtctgccgccgctgaagtagtgagccgtaatcagcaagagttactgcgccaggtgcaatcgacccgtcaacggctggagatggcgatggattattatctcgccaaccgcacacgtcgctttacgcaaattcatcaccgattacagcaacagcatccgcagctccggctggcacgccagcaaaccatgcttgagcgcctgcaaaagcgaatgagctttgcgctggaaaatcaacttaagcgtaccgggcaacagcagcagcggttaacacagcggctgaatcagcaaaatccacagccgaagattcatcgcgcgcaaacgcgcattcagcaactggaatatcgtttagcagaaaccctgcgcgcacagcttagcgccacgcgtgaacgtttcggtaatgcagtaacgcacctcgaagccgtaagcccactgtcaacgctggcgcgtggatacagcgttactactgctactgacggcaatgtactgaaaaaagtgaagcaagttaaagcgggtgaaatgctaaccacacgtctggaagacggctggatagaaagtgaagtaaaaaacatccagccagtaaaaaaatcgcgtaaaaaggtgcattaa

SEQ ID NO: 67 - DNA, 1736 bp, artificial sequence
  recJ (Escherichia coli)
gtgaaacaacagatacaacttcgtcgtgacgtgaagtcgatgaaacggcagacttgcccgctgaattgcctcccttgctgcgccgtttatacgccagccggggagtacgcagtgcgcaagaactggaacgcagtgttaaaggtatgctgccctggcagcaactgagcggcgtcgaaaaggccgttgagatcctttacaacgcttttcgcgaaggaacgcggattattgtggtcggtgatttcgacgccgacggcgcgaccagcacggctctaagcgtgctggcgatgcgctcgcttggttgcagcaatatcgactacctggtaccaaaccgtttcgaagacggttacggcttaagcccggaagtggtcgatcaggcccatgcccgtggcgcgcagttaattgtcacggtggataacggtatttcctcccatgcgggggttgagcacgctcgctcgttgggcatcccggttattgttaccgatcaccatttgccaggcgacacattacccgcagcggaagcgatcattaaccctaacttgcgcgactgtaatttcccgtcgaaatcactggcaggcgtgggtgtggcgttttatctgatgctggcgctgcgcacctttttgcgcgatcagggctggtttgatgagcgtaacatcgcaattcctaacctggcagaactgctggatctggtcgcgctggggacagtggcggacgtcgtgccgctggacgctaataatcgcattctgacctggcaggggatgagtcgcatccgagccggaaagtgccgtccggggattaaagcgctgcttgaagtggcaaaccgtgatgcacaaaaactcgccgccagcgatttaggttttgcgctggggccacgtctcaatgctgccggacgactggacgatatgtccgtcggtgtggcgctgttgttgtgcgacaacatcggcgaagcgcgcgtgctggcaaatgaactcgatgcgctaaaccagacgcgaaaagagatcgaacaaggaatgcaaattgaagccctgaccctgtgcgagaaactggagcgcagccgtgacacgctacccggcgggctggcaatgtatcaccccgaatggcatcagggcgttgtcggtattctggcttcgcgcatcaaagagcgttttcaccgtccggttatcgcgtttgcgccagcaggtgacggtacgctgaaaggttccggtcgctccattcaggggctgcatatgcgtgatgcgctggagcgattagacacactctaccctggcatgatgctgaagtttggcggtcatgcgatggcggcgggtttgtcgctggaagaggataaattcaaactctttcaacaacggtttggcgaactggttactgagtggctggacccttcgctattgcaaggcgaagtggtatcagacggtccgttaagcccggccgaaatgaccatggaagtggcgcagctgctgcgcgatgctggcccgtgggggcagatgttcccggagccgctgtttgacggtcatttccgtctgctgcaacagcggctggtgggcgaacgtcatttgaaggtgatggtcgaaccggtcggcggcggtccactgctggatggtattgcttttaatgtcgataccgccctctggccggataacggcgtgcgcgaagtgcaactggcttataagctcgatatcaacgagtttcgcggcaaccgcagcctgcaaattatcatcgacaatatctggccaatttag

SEQ ID NO: 68 - DNA, 43 bp, artificial
  part of promoter
ggcggcttgacactttatgcttccggctcggatactgtgtgga

SEQ ID NO: 69 - DNA, 40 bp, artificial
  transcription terminator
cgcaaaaaaccccgcccctgacagggcggggttttttcgc

SEQ ID NO: 70 - DNA, 66 bp, artificial
  strong promoter with weak RBS
ttgacatccctatcagtgatagagatactgctagcacttaagtagaccagctcgctaggtcatata

SEQ ID NO: 71 - DNA, 320 bp, artificial
  NGS Full amplicon sequence double tagged
  misc_feature (74)..(80): HHNHHNH corresponds to 3888 sequences
  misc_feature (245)..(251): DNDDNDD corresponds to 3888 sequences
aatgatacggcgaccaccgagatctacactaatcttaacactctttccctacacgacgctcttccgatctcgchhnhhnhattcggaagctttcgttgacttacgtgatgtacgtcagcctgaagtgaaagaagagaaaccagaggcggccgcagccaagttgaccagtgcagttccggtgctcaccgcgcgcgacgtcgccggagcggtcgagttctggaccgaccggctcgggttctcacctdnddnddgcgagatcggaagagcacacgtctgaactccagtcaccttgctagatctcgtatgccgtcttctgcttg

SEQ ID NO: 72 - DNA, 97 bp, artificial
  PRIMER BINDING SITE 5′fragment
  misc_feature (76)..(76): n is a, c, g, or t
  misc_feature (79)..(79): n is a, c, g, or t
aatgatacggcgaccaccgagatctacactaatcttaacactctttccctacacgacgctcttccgatctcgchhnhhnhattctgagacctttccc

SEQ ID NO: 73 - DNA, 93 bp, artificial
  Primer Binding site 3′fragment
  misc_feature (19)..(19): n is a, c, g, or t
  misc_feature (22)..(22): n is a, c, g, or t
gggaaaggtctcaacctdnddnddgcgagatcggaagagcacacgtctgaactccagtcaccttgctagatctcgtatgccgtcttctgcttg

SEQ ID NO: 74 - DNA, 190 bp, artificial
  Primer Binding Site internal fragment
gggaaaggtctcaattcggaagctttcgttgacttacgtgatgtacgtcagcctgaagtgaaagaagagaaaccagaggcggccgcagccaagttgaccagtgcagttccggtgctcaccgcgcgcgacgtcgccggagcggtcgagttctggaccgaccggctcgggttctcaccttgagacctttccc

SEQ ID NO: 75 - DNA, 38 bp, artificial
  OR2004  Internal fragment forward primer
gggaaaggtctcaattcggaagctttcgttgacttacg

SEQ ID NO: 76 - DNA, 32 bp, artificial
  OR2005   Internal fragment forward primer
gggaaaggtctcaaggtgagaacccgagccgg